(19) United States
(12) Patent Application Publication
(10) Pub. No.: US 2024/0183845 A1
(43) Pub. Date: Jun. 6, 2024

(54) BILE ACID METABOLITES FOR DIAGNOSING AND TREATING DEPRESSIVE DISORDERS

(71) Applicants: Rima F. KADDURAH-DAOUK, Belmont, MA (US); Augustus John RUSH, Durham, NC (US); Siamak MAHMOUDIANDEHKORDI, Durham, NC (US); Sudeepa Bhatacharyya, Jonesboro, AR (US); Boadie W. Dunlap, Atlanta, GA (US); W. Edward CRAIGHEAD, Atlanta, GA (US); Helen S. Mayberg, Atlanta, GA (US); Duke University, Durham, NC (US); Emory University, Atlanta, GA (US)

(72) Inventors: Rima F. Kaddurah-Daouk, Belmont, MA (US); Augustus John Rush, Jr., Durham, NC (US); Siamak MahmoudianDehkordi, Durham, NC (US); Sudeepa Bhattacharyya, Jonesboro, AR (US); Boadie W. Dunlap, Atlanta, GA (US); W. Edward Craighead, Atlanta, GA (US); Helen S. Mayberg, Atlanta, GA (US)

(21) Appl. No.: 18/554,830

(22) PCT Filed: Jun. 8, 2022
(86) PCT No.: PCT/US2022/032734

§ 371 Date: Oct. 11, 2023

Related U.S. Application Data
(60) Provisional application No. 63/175,276, filed on Apr. 15, 2021.

(51) Int. Cl. G01N 33/53; A61K 31/343; A61K 31/381; A61P 25/24; C12Q 1/6883

(52) U.S. Cl. CPC G01N 33/5308; A61K 31/343; A61K 31/381; A61P 25/24; C12Q 1/6883; C12Q 2600/156; G01N 2800/301; G01N 2800/304; G01N 2800/52

(57) ABSTRACT

Described herein are methods and compositions for diagnosing and evaluating the treatment of depression using one or more biomarker metabolites for the diagnosis and monitoring treatment efficacy. In one aspect, the biomarker metabolites comprise bile acids and can be used to screen subjects for the likelihood of developing depression or anxiety, the diagnosis thereof, monitoring the efficacy of treatment, and evaluating a subject's propensity for responding to treatment.

### CROSS-REFERENCE TO RELATED APPLICATIONS

This application claims priority to U.S. Provisional Patent Application No. 63/175,276, filed on Apr. 15, 2021, which is incorporated by reference herein in its entirety.

### FEDERALLY SPONSORED RESEARCH

This invention was made with government support under grant numbers MH108348 awarded by the National Institutes of Health. The government has certain rights in the invention.

### TECHNICAL FIELD

Described herein are methods and compositions for diagnosing and evaluating the treatment of depression, anxiety, or neuropsychiatric disorders using one or more biomarker metabolites for the diagnosis and monitoring treatment efficacy. In one aspect, the biomarker metabolites comprise bile acids and can be used to screen subjects for the likelihood of developing depression or anxiety, the diagnosis thereof, monitoring the efficacy of treatment, and evaluating a subject's propensity for responding to treatment.

### BACKGROUND

The gut microbiome may play a role in the pathogenesis of neuropsychiatric diseases including major depressive disorder (MDD), anxiety, or other mood disorders. Bile acids (BAs) are steroid acids that are synthesized in the liver from cholesterol and further processed by gut-bacterial enzymes, thus requiring both human and gut microbiome enzymatic processes in their metabolism. BAs participate in a range of important host functions such as lipid transport and metabolism, cellular signaling and regulation of energy homeostasis. BAs have recently been implicated in the pathophysiology of Alzheimer's and several neuropsychiatric diseases, but the biochemical underpinnings of these gut microbiome-linked metabolites in the pathophysiology of depression and anxiety remains largely unknown.

A potential mechanism by which the gut microbiome may alter CNS function is its impact on BAs. BAs are the amphipathic end products of cholesterol metabolism and can contribute significantly to hepatic, intestinal, and metabolic disorders. FIG. 1 shows how BAs are synthesized from cholesterol in the liver by two major pathways, the classical and the alternative, and metabolized into secondary BAs by colonic bacteria through multiple and well-characterized enzymatic pathways. Primary BAs are the direct products of cholesterol metabolites in hepatocytes, such as cholic acid (CA) and chenodeoxycholic acid (CDCA). In response to cholecystokinin after feeding, primary BAs are secreted by the liver into the small intestine to ensure absorption of dietary lipids. Accordingly, 95% of the BAs are actively absorbed in the terminal ileum and redirected into the portal circulation to reenter the liver. A small proportion pass into the colon where they are transformed by bacteria into secondary BAs-lithocholic acid (LCA), deoxycholic acid (DCA), and ursodeoxycholic acid-via deconjugation and 7α-dehydroxylation, among other metabolites. Although DCA and LCA are the most abundant secondary BAs, around 50 different secondary BAs have been detected in human feces.

Hepatic encephalopathy is associated with elevated levels of ammonia and cytotoxic BAs, including several conjugated primary and secondary BAs. Post-mortem brain samples and serum concentrations of living Alzheimer's disease patients demonstrate lower levels of the primary bile acid, CA, and higher levels of its bacterially-derived secondary bile acid, DCA and its conjugated forms compared to matched controls. In contrast, ursodeoxycholic acid, the 7B isomer of CDCA, has antiapoptotic, anti-inflammatory, antioxidant, and neuroprotective effects in various models of neurodegenerative diseases and Huntington's disease. Taken together, these data indicate that BAs affect brain function under both normal and pathological conditions. However, the association of BAs on psychiatric diseases such as MDD has received little study to date.

What is needed are methods for using bile acid biomarker metabolites for detecting and evaluating the treatment of depression and anxiety and for monitoring treatment efficacy. Specific bile acid compositions may be useful as supplements for treating depression and anxiety. Drugs Targeting the gut microbiome and production of bile acids could become an important added strategy for the treatment of depression anxiety and neuropsychiatric diseases where SSRIs might not work. Modulation of gut microbiome could be achieved with use of probiotics, with targeting bacterial enzymes, genetically and chemically to change gut microbiome composition and activity among other approaches. The primary bile acids are used by bacteria to produce toxic compounds these can be modulated by regulation of human enzymes and liver function.

### SUMMARY

One embodiment described herein is a method for the classification and treatment of depression, anxiety, mood disorders, or neuropsychiatric symptoms in a subject based on the subject's metabolic profile and genetic screening, the method comprising one or more of the following: identifying and stratifying subjects afflicted with a depression, anxiety, mood disorders, or neuropsychiatric symptoms to subgroups based on their metabolic profiles, biomarker metabolites and ratios of biomarker metabolites that define unique metabolic conditions related to aberrations in cholesterol metabolism or bile acid anabolism, catabolism homeostasis, or clearance and common identity among subgroups of subjects; evaluating the trajectory of disease within each stratified subgroup of subjects and their response to a therapeutic treatment; identifying defects in bile acid transport and/or biosynthesis of biomarker metabolites within a metabolic pathway or across metabolic pathways using ratios of biomarker metabolites to inform about changes in enzyme activities or transporters; and identifying genetic bases of bile acid metabolic profile characteristics or defects (SNPs/genetic variants in key enzymes and transporters) using metabolite genome-wide association study (mGWAS) analysis. In one aspect, the method further comprises one or more of the following: using combined metabotype and genotype data to better stratify subjects with depression, anxiety, mood disorders, or neuropsychiatric symptoms and to inform about mechanisms and treatment selection; suggesting a therapeutic approach to correct metabolic defects in metabolic profile in stratified subgroups of subjects; and comparing and contrasting metabolic defects noted in inborn errors of metabolism that have depression, anxiety, mood disorders, or neuropsychiatric symptoms and using knowledge gained in treatment of inborn errors of metabolism to inform treatment for depression, anxiety, mood disorders, or neuropsychiatric symptoms. In another aspect, the method further comprises performing imaging analysis on the subject and linking peripheral changes with brain changes. In another aspect, the method further comprises administering to the subjects an effective amount of a therapy to prevent and/or treat the depression, anxiety, mood disorders, or neuropsychiatric symptoms affected by one or more metabolic defects. In another aspect, the depression, anxiety, mood disorders, or neuropsychiatric symptoms are associated with neurological diseases or cognitive impairment, including dementia, vascular dementia, mixed dementia, early mild cognitive impairment (EMCI), late mild cognitive impairment (LMCI), Alzheimer's Disease, dementia with Lewy bodies, frontotemporal dementia, Creutzfeldt-Jakob disease, Parkinson's Disease, young-onset dementia, Korsakoff's syndrome, Huntington's disease, HIV-associated neurocognitive disorders, or other cognitive impairment disorders.

Another embodiment described herein is a method for stratifying and treating a subject having a depression, anxiety, mood disorders, or neuropsychiatric symptoms, or at risk of developing a neurological disorder, based on the subject's metabolic profile, the method comprising: analyzing a sample from a subject to determine concentration levels or ratios of one or more biomarker metabolites or gut microbiome-related biomarker metabolites related to bile acid anabolism, catabolism or homeostasis in the sample from the subject; determining if the subject has a metabolic defect related to disrupted bile acid anabolism, catabolism or homeostasis, or if the subject's gut microbiome has a defect related to disrupted bile acid anabolism, catabolism or homeostasis, or combinations thereof based on the measured concentration levels and calculated ratios of the one or more bile acid anabolism, catabolism or homeostasis biomarker metabolites in the sample as compared to a control sample; stratifying the subject into a subgroup of subjects, wherein an individual subgroup of subjects is defined by a unique and specific bile acid anabolism, catabolism or homeostasis profile based on the measured concentration levels and calculated ratios of the one or more biomarker metabolites or gut microbiome-related biomarker metabolites in the sample as compared to a control sample and the biomarker metabolite or gut microbiome-related biomarker metabolite defect determined for the subject. In one aspect, the method further comprises treating the depression, anxiety, mood disorders, or neuropsychiatric symptoms by administering to the subgroup of subjects an effective amount sufficient to attenuate, reduce, or eliminate the symptoms of depression, anxiety, mood disorders, or neuropsychiatric symptoms of a therapy to wherein the therapy is determined by the unique and specific metabolic profile of the subgroup of subjects. In another aspect, the one or more biomarker metabolites or gut microbiome-related biomarker metabolites comprises one or more of: Primary Bile Acids: cholic acid (CA); chenodeoxycholic acid (CDCA); chenodeoxycholic acid-24-acyl-β-d-glucuronide (CDCA-24G); 3β-cholic acid (β-CA); hyocholic acid (HCA); ω-monocarboxylic acid (ω-MCA); taurocholic acid (TCA); glycocholic acid (GCA); taurochenodeoxycholate (TCDCA); glycochenodeoxycholate (GCDCA); 5β-cholanic acid-3β,7β,12α-triol-5β-cholanic acid-3β, 7β, 12α-triol (β-UCA); taurohyocholate (THCA); glycohyocholate (GHCA); Secondary Bile Acids: glycohyodeoxycholate (GHDCA); deoxycholic acid (DCA); 23-nordeoxycholic acid (NorDCA); β-ursodeoxycholic acid (UDCA); lithocholic acid (LCA); 6,7-diketolithocholic acid (6,7-diketoLCA); 7-ketolithocholic acid (7-ketoLCA); lithocholic acid-3-sulfate (LCA-3S); hyodeoxycholic acid (HDCA); isolithocholic acid (isoLCA); β-hyodeoxycholic acid (isohyodeoxycholic acid; β-HDCA); allolithocholic acid (isoallolithocholic acid; alloLCA); dehydroLCA; 12-ketodeoxycholic acid (12-ketoLCA); 3β-ursodeoxycholic acid (isoursodeoxycholic acid; 3β-UDCA); glycodeoxycholic acid (GDCA); tauroursodeoxycholic acid (TUDCA); glycoursodeoxycholic acid (GUDCA); taurodeoxycholic acid (TDCA); glycolithocholic acid-3-sulfate (GLCA-3S); glycolithocholate (GLCA); taurohyodeoxycholic acid (THDCA); norcholic acid (NorCA); conjugated forms of typical bile acids; or combinations thereof. In another aspect, the method further comprises: administering to the subject a therapeutically effective amount of one or more primary or secondary bile acids and/or any pharmaceutically acceptable derivatives, esters, salts, solvates, hydrates, analogs, or prodrugs thereof; and/or administering to the subject a therapeutically effective amount of one or more therapeutic agents capable of modulating (increasing or decreasing) the concentration levels or ratios of one or more primary or secondary bile acids, activating the endogenous production of one or more primary or secondary bile acids, and/or decreasing the breakdown of one or more primary or secondary bile acids; and/or administering to the subject a therapeutically effective amount of one or more antidepressants selected from citalopram (Celexa®), escitalopram (Lexapro®), duloxetine (Cymbalta®), fluoxetine (Prozac®), paroxetine (Paxil®), sertraline (Zoloft®), trazodone (Desyrel®), lorazepam (Ativan®), oxazepam (Serax®), fluvoxamine (Luvox®), vilazodone (Viibryd®), vortioxetine (Trintellix®), aripiprazole (Abilify®), clozapine (Clozaril®), haloperidol (Haldol®), olanzapine (Zyprexa®), quetiapine (Seroquel®), risperidone (Risperdal®), ziprasidone (Geodon®), amitriptyline, amoxapine, desipramine (Norpramin®), doxepin, imipramine (Tofranil®), nortriptyline (Pamelor®), protriptyline, trimipramine, ketamine, or combinations thereof. In another aspect, the depression, anxiety, mood disorders, or neuropsychiatric symptoms are associated with neurological diseases or cognitive impairment, including dementia, vascular dementia, mixed dementia, early mild cognitive impairment (EMCI), late mild cognitive impairment (LMCI), Alzheimer's Disease, dementia with Lewy bodies, frontotemporal dementia, Creutzfeldt-Jakob disease, Parkinson's Disease, young-onset dementia, Korsakoff's syndrome, Huntington's disease, HIV-associated neurocognitive disorders, or other cognitive impairment disorders.

Another embodiment described herein is a method for detecting depression or anxiety in a subject, the method comprising: analyzing a sample from a subject; determining concentration levels or ratios of one or more biomarker metabolites or gut microbiome-related biomarker metabolites in the sample from the subject; and determining the subject as having depression or anxiety or an increased risk of depression or anxiety when the concentration levels or ratios of the one or more biomarker metabolites or gut microbiome-related biomarker metabolites in the sample from the subject are different from (greater than or less than) the concentration levels or ratios of the one or more biomarker metabolites or gut microbiome-related biomarker metabolites in a control sample. In another aspect, the method further comprises: initially treating the subject for depression or anxiety by administering an effective amount sufficient to attenuate, reduce, or eliminate the symptoms of depression or anxiety of one or more of antidepressants, cognitive behavior therapy, exercise, dietary supplements, prebiotics, probiotics, dietary changes, or an elimination diet; obtaining a second sample from the subject and determining the concentration levels or ratios of one or more biomarker metabolites or gut microbiome-related biomarker metabolites in the second sample from the subject; evaluating the concentration levels or ratios of the one or more biomarker metabolites or gut microbiome-related biomarker metabolites in comparison to control concentration levels or ratios of the one or more biomarker metabolites or gut microbiome-related biomarker metabolites; evaluating the efficacy of the depression or anxiety treatment; and continuing the one or more initial depression or anxiety treatments; administering one or more additional depression or anxiety treatments; or administering one or more second depression or anxiety treatments (switching the treatment regimen). In another aspect, the one or more biomarker metabolites or gut microbiome-related biomarker metabolites comprises one or more of: Primary Bile Acids: cholic acid (CA); chenodeoxycholic acid (CDCA); chenodeoxycholic acid-24-acyl-β-d-glucuronide (CDCA-24G); 3β-cholic acid (β-CA); hyocholic acid (HCA); ω-monocarboxylic acid (ω-MCA); taurocholic acid (TCA); glycocholic acid (GCA); taurochenodeoxycholate (TCDCA); glycochenodeoxycholate (GCDCA); 5β-cholanic acid-3β,7β,12α-triol-5β-cholanic acid-3β, 7β, 12α-triol (β-UCA): taurohyocholate (THCA); glycohyocholate (GHCA); Secondary Bile Acids: glycohyodeoxycholate (GHDCA); deoxycholic acid (DCA); 23-nordeoxycholic acid (NorDCA); β-ursodeoxycholic acid (UDCA); lithocholic acid (LCA); 6,7-diketolithocholic acid (6,7-diketoLCA); 7-ketolithocholic acid (7-ketoLCA); lithocholic acid-3-sulfate (LCA-3S); hyodeoxycholic acid (HDCA); isolithocholic acid (isoLCA); β-hyodeoxycholic acid (isohyodeoxycholic acid; β-HDCA); allolithocholic acid (isoallolithocholic acid; alloLCA); dehydroLCA; 12-ketodeoxycholic acid (12-ketoLCA); 3β-ursodeoxycholic acid (isoursodeoxycholic acid; 3β-UDCA); glycodeoxycholic acid (GDCA); tauroursodeoxycholic acid (TUDCA); glycoursodeoxycholic acid (GUDCA); taurodeoxycholic acid (TDCA); glycolithocholic acid-3-sulfate (GLCA-3S); glycolithocholate (GLCA); taurohyodeoxycholic acid (THDCA); norcholic acid (NorCA); conjugated forms of typical bile acids; or combinations thereof. In another aspect, the method further comprises: administering to the subject a therapeutically effective amount of one or more primary or secondary bile acids and/or any pharmaceutically acceptable derivatives, esters, salts, solvates, hydrates, analogs, or prodrugs thereof; and/or administering to the subject a therapeutically effective amount of one or more therapeutic agents capable of modulating (increasing or decreasing) the concentration levels or ratios of one or more primary or secondary bile acids, activating the endogenous production of one or more primary or secondary bile acids, and/or decreasing the breakdown of one or more primary or secondary bile acids. In another aspect, the biomarker metabolite or gut microbiome-related biomarker metabolite concentration level is greater than the control concentration level. In another aspect, the biomarker metabolite or gut microbiome-related biomarker metabolite concentration level is less than the control concentration level. In another aspect, two or more biomarker metabolite or gut microbiome-related biomarker metabolite concentration levels covary and are greater than the control concentration levels or covary and are less than the control concentration levels (positive correlation). In another aspect, two or more biomarker metabolite or gut microbiome-related biomarker metabolite concentration levels vary dissimilarly compared to the control concentration levels (negative correlation). In another aspect, the biomarker metabolite or gut microbiome-related biomarker metabolite comprises one or more primary bile acids selected from the group consisting of CDCA, CA, HCA, GHCA, and combinations thereof. In another aspect, when the biomarker metabolite or gut microbiome-related biomarker metabolite comprises CDCA, the concentration levels in the sample from the subject are less than the concentration levels in the control sample. In another aspect, the biomarker metabolite or gut microbiome-related biomarker metabolite comprises one or more secondary bile acids selected from the group consisting of UDCA, LCA, 7-ketoLCA, HDCA, isoLCA, β-HDCA, alloLCA, dehydroLCA, 12-ketoLCA, LCA-3S, GLCA-3S, HCA, NorCA, 6,7-diketoLCA, GDCA, β-UCA, and combinations thereof. In another aspect, when the biomarker metabolite or gut microbiome-related biomarker metabolite comprises one or more of LCA, isoLCA, alloLCA, 12-ketoLCA, LCA-3S, GLCA-3S, 7-ketoLCA, 6,7-diketoLCA, NorCA, GDCA, or β-HDCA, the concentration levels in the sample from the subject are greater than the concentration levels in the control sample. In another aspect, when the biomarker metabolite or gut microbiome-related biomarker metabolite comprises one or more of dehydroLCA, β-UCA, or HCA, the concentration levels in the sample from the subject are less than the concentration levels in the control sample. In another aspect, one or more ratios of the biomarker metabolites or gut microbiome-related biomarker metabolites are determined, the one or more ratios comprising a ratio of LCA/CDCA, 7-ketoLCA/CDCA, 12-ketoLCA/CDCA, GHCA/CDCA, HDCA/CDCA, ω-MCA/CDCA, alloLCA/CDCA, isoLCA/CDCA, LCA-3S/CDCA, GCA/TCA, TDCA/GDCA, THCA/GHCA, or combinations thereof. In another aspect, when the ratio of the biomarker metabolites or gut microbiome-related biomarker metabolites comprises one or more of LCA/CDCA, 7-ketoLCA/CDCA, 12-ketoLCA/CDCA, GHCA/CDCA, HDCA/CDCA, ω-MCA/CDCA, alloLCA/CDCA, isoLCA/CDCA, LCA-3S/CDCA, or GCA/TCA, the ratios in the sample from the subject are greater than the ratios in the control sample. In another aspect, when the ratio of the biomarker metabolites or gut microbiome-related biomarker metabolites comprises one or more of TDCA/GDCA or THCA/GHCA, the ratios in the sample from the subject are less than the ratios in the control sample. In another aspect, the sample from the subject is selected from one or more of whole blood, serum, plasma, urine, saliva, feces, or other body fluids. In another aspect, the control sample is from an untreated subject or a subject or a population of subjects not experiencing depression, anxiety, mood disorders, or neuropsychiatric symptoms or not at risk for depression, anxiety, mood disorders, or neuropsychiatric symptoms. In another aspect, the depression is Major Depression Disorder (MDD), core depression (CD+), anxious depression (ANX+), neurovegetative symptoms of melancholia (NVSM+), treatment resistant depression, subclinical characteristics associated with depression, or a neuropsychiatric symptom associated with a neurological disease or cognitive impairment. In another aspect, the depression, anxiety, mood disorders, or neuropsychiatric symptoms are associated with neurological diseases or cognitive impairment, including dementia, vascular dementia, mixed dementia, early mild cognitive impairment (EMCI), late mild cognitive impairment (LMCI), Alzheimer's Disease, dementia with Lewy bodies, frontotemporal dementia, Creutzfeldt-Jakob disease, Parkinson's Disease, young-onset dementia, Korsakoff's syndrome, Huntington's disease, HIV-associated neurocognitive disorders, or other cognitive impairment disorders. In another aspect, the antidepressant comprises citalopram (Celexa®), escitalopram (Lexapro®), duloxetine (Cymbalta®), fluoxetine (Prozac®), paroxetine (Paxil®), sertraline (Zoloft®), trazodone (Desyrel®), lorazepam (Ativan®), oxazepam (Serax®), fluvoxamine (Luvox®), vilazodone (Viibryd®), vortioxetine (Trintellix®), aripiprazole (Abilify®), clozapine (Clozaril®), haloperidol (Haldol®), olanzapine (Zyprexa®), quetiapine (Seroquel®), risperidone (Risperdal®), ziprasidone (Geodon®), amitriptyline, amoxapine, desipramine (Norpramin®), doxepin, imipramine (Tofranil®), nortriptyline (Pamelor®), protriptyline, trimipramine, ketamine, or combinations thereof. In another aspect, the antidepressant is one or more selective serotonin reuptake inhibitors (SSRI). In another aspect, the antidepressant comprises citalopram (Celexa®), escitalopram (Lexapro®), duloxetine (Cymbalta®), fluoxetine (Prozac®), paroxetine (Paxil®), sertraline (Zoloft®), paroxetine (Paxil®), fluvoxamine (Luvox®), vilazodone (Viibryd®), vortioxetine (Trintellix®), or other SSRI. In another aspect, the antidepressant comprises ketamine. In another aspect, the efficacy of the depression treatment is evaluated using the Hamilton Depression Rating Scale (HRSD₁₇), the Quick Inventory of Depressive Symptomatology (QIDS), subscales thereof, or specific questions thereof.

Another embodiment described herein is a method for treating depression, anxiety, mood disorders, or neuropsychiatric symptoms in a subject the method comprising: administering to the subject an effective amount sufficient to attenuate, reduce, or eliminate the symptoms of depression, anxiety, mood disorders, or neuropsychiatric symptoms of one or more of antidepressants, cognitive behavior therapy, exercise, dietary supplements, prebiotics, probiotics, dietary changes, or an elimination diet; analyzing sample from the subject; and measuring concentration levels or ratios in the subject's sample of one or more biomarker metabolites or gut microbiome-related biomarker metabolites comprising primary bile acids, secondary bile acids, or a combination thereof. In one aspect, the one or more biomarker metabolites or gut microbiome-related biomarker metabolites comprises one or more of: Primary Bile Acids: cholic acid (CA); chenodeoxycholic acid (CDCA); chenodeoxycholic acid-24-acyl-β-d-glucuronide (CDCA-24G); 3β-cholic acid (β-CA); hyocholic acid (HCA); ω-monocarboxylic acid (ω-MCA); taurocholic acid (TCA); glycocholic acid (GCA); taurochenodeoxycholate (TCDCA); glycochenodeoxycholate (GCDCA); 5β-cholanic acid-3β, 7β, 12α-triol-5β-cholanic acid-3β, 7β, 12α-triol (β-UCA); taurohyocholate (THCA): glycohyocholate (GHCA); Secondary Bile Acids: glycohyodeoxycholate (GHDCA); deoxycholic acid (DCA); 23-nordeoxycholic acid (NorDCA); β-ursodeoxycholic acid (UDCA); lithocholic acid (LCA); 6,7-diketolithocholic acid (6,7-diketoLCA); 7-ketolithocholic acid (7-ketoLCA); lithocholic acid-3-sulfate (LCA-3S); hyodeoxycholic acid (HDCA); isolithocholic acid (isoLCA); β-hyodeoxycholic acid (isohyodeoxycholic acid; β-HDCA); allolithocholic acid (isoallolithocholic acid; alloLCA); dehydroLCA; 12-ketodeoxycholic acid (12-ketoLCA); 3β-ursodeoxycholic acid (isoursodeoxycholic acid; 3β-UDCA); glycodeoxycholic acid (GDCA); tauroursodeoxycholic acid (TUDCA); glycoursodeoxycholic acid (GUDCA); taurodeoxycholic acid (TDCA); glycolithocholic acid-3-sulfate (GLCA-3S); glycolithocholate (GLCA); taurohyodeoxycholic acid (THDCA); norcholic acid (NorCA); conjugated forms of typical bile acids; or combinations thereof. In another aspect, the method further comprises: administering to the subject a therapeutically effective amount of one or more primary or secondary bile acids and/or any pharmaceutically acceptable derivatives, esters, salts, solvates, hydrates, analogs, or prodrugs thereof; and/or administering to the subject a therapeutically effective amount of one or more therapeutic agents capable of modulating (increasing or decreasing) the concentration levels or ratios of one or more primary or secondary bile acids, activating the endogenous production of one or more primary or secondary bile acids, and/or decreasing the breakdown of one or more primary or secondary bile acids. In another aspect, the depression, anxiety, mood disorders, or neuropsychiatric symptoms are associated with neurological diseases or cognitive impairment, including dementia, vascular dementia, mixed dementia, early mild cognitive impairment (EMCI), late mild cognitive impairment (LMCI), Alzheimer's Disease, dementia with Lewy bodies, frontotemporal dementia, Creutzfeldt-Jakob disease, Parkinson's Disease, young-onset dementia, Korsakoff's syndrome, Huntington's disease, HIV-associated neurocognitive disorders, or other cognitive impairment disorders.

Another embodiment described herein is a method of treating depression, anxiety, mood disorders, or neuropsychiatric symptoms in a subject, the method comprising: administering to the subject an effective amount sufficient to attenuate, reduce, or eliminate the symptoms of depression, anxiety, mood disorders, or neuropsychiatric symptoms of one or more primary or secondary bile acids and/or any pharmaceutically acceptable derivatives, esters, salts, solvates, hydrates, analogs, or prodrugs thereof; and/or administering to the subject a therapeutically effective amount of one or more therapeutic agents capable of modulating (increasing or decreasing) the concentration levels or ratios of one or more primary or secondary bile acids, activating the endogenous production of one or more primary or secondary bile acids, and/or decreasing the breakdown of one or more primary or secondary bile acids. In one aspect, the depression, anxiety, mood disorders, or neuropsychiatric symptoms are associated with neurological diseases or cognitive impairment, including dementia, vascular dementia, mixed dementia, early mild cognitive impairment (EMCI), late mild cognitive impairment (LMCI), Alzheimer's Disease, dementia with Lewy bodies, frontotemporal dementia, Creutzfeldt-Jakob disease, Parkinson's Disease, young-onset dementia, Korsakoff's syndrome, Huntington's disease, HIV-associated neurocognitive disorders, or other cognitive impairment disorders.

Another embodiment described herein is a method for detecting and treating depression, anxiety, mood disorders, or neuropsychiatric symptoms in a subject, the method comprising: analyzing a sample from a subject; measuring concentration levels or ratios of one or more biomarker metabolites or gut microbiome-related biomarker metabolites comprising primary bile acids, secondary bile acids, or a combination thereof in the sample from the subject; determining the subject as having depression, anxiety, mood disorders, or neuropsychiatric symptoms or an increased risk of depression, anxiety, mood disorders, or neuropsychiatric symptoms when the concentration levels or ratios of the one or more biomarker metabolites or gut microbiome-related biomarker metabolites in the sample from the subject are different from (greater than or less than) the concentration levels or ratios of the one or more biomarker metabolites or gut microbiome-related biomarker metabolites in a control sample; and treating the subject by administering an effective amount sufficient to attenuate, reduce, or eliminate the symptoms of depression, anxiety, mood disorders, or neuropsychiatric symptoms of one or more primary or secondary bile acids and/or any pharmaceutically acceptable derivatives, esters, salts, solvates, hydrates, analogs, or prodrugs thereof, and/or administering an effective amount of one or more therapeutic agents capable of modulating (increasing or decreasing) the concentration levels or ratios of one or more primary or secondary bile acids, activating the endogenous production of one or more primary or secondary bile acids, and/or decreasing the breakdown of one or more primary or secondary bile acids. In one aspect, the one or more biomarker metabolites or gut microbiome-related biomarker metabolites comprises one or more of: Primary Bile Acids: cholic acid (CA); chenodeoxycholic acid (CDCA); chenodeoxycholic acid-24-acyl-β-d-glucuronide (CDCA-24G): 3β-cholic acid (β-CA); hyocholic acid (HCA); ω-monocarboxylic acid (ω-MCA); taurocholic acid (TCA); glycocholic acid (GCA); taurochenodeoxycholate (TCDCA); glycochenodeoxycholate (GCDCA); 5β-cholanic acid-3β,7β,12α-triol-5β-cholanic acid-3β, 7β, 12α-triol (β-UCA); taurohyocholate (THCA); glycohyocholate (GHCA); Secondary Bile Acids: glycohyodeoxycholate (GHDCA); deoxycholic acid (DCA); 23-nordeoxycholic acid (NorDCA); β-ursodeoxycholic acid (UDCA); lithocholic acid (LCA); 6,7-diketolithocholic acid (6,7-diketoLCA); 7-ketolithocholic acid (7-ketoLCA); lithocholic acid-3-sulfate (LCA-3S); hyodeoxycholic acid (HDCA); isolithocholic acid (isoLCA); β-hyodeoxycholic acid (isohyodeoxycholic acid; β-HDCA); allolithocholic acid (isoallolithocholic acid; alloLCA); dehydroLCA; 12-ketodeoxycholic acid (12-ketoLCA); 3β-ursodeoxycholic acid (isoursodeoxycholic acid; 3β-UDCA); glycodeoxycholic acid (GDCA); tauroursodeoxycholic acid (TUDCA); glycoursodeoxycholic acid (GUDCA); taurodeoxycholic acid (TDCA); glycolithocholic acid-3-sulfate (GLCA-3S); glycolithocholate (GLCA); taurohyodeoxycholic acid (THDCA); norcholic acid (NorCA); conjugated forms of typical bile acids; or combinations thereof. In another aspect, the depression, anxiety, mood disorders, or neuropsychiatric symptoms are associated with neurological diseases or cognitive impairment, including dementia, vascular dementia, mixed dementia, early mild cognitive impairment (EMCI), late mild cognitive impairment (LMCI), Alzheimer's Disease, dementia with Lewy bodies, frontotemporal dementia, Creutzfeldt-Jakob disease, Parkinson's Disease, young-onset dementia, Korsakoff's syndrome, Huntington's disease, HIV-associated neurocognitive disorders, or other cognitive impairment disorders.

Another embodiment described herein is a method for isolating a biomarker metabolite useful for the analysis and identification of metabolic changes associated with depression, anxiety, mood disorders, or neuropsychiatric symptoms in a subject, the method comprising: analyzing a sample from a subject and a control subject or population of subjects with normal cognition; isolating one or more biomarker metabolites or gut microbiome-related biomarker metabolites in the sample from the subject and the control sample, wherein the one or more biomarker metabolites or gut microbiome-related biomarker metabolites are selected from the group consisting of a primary bile acid, a secondary bile acid, and combinations thereof; detecting and measuring concentration levels or ratios of the one or more biomarker metabolites or gut microbiome-related biomarker metabolites in the sample from the subject and the control sample using biochemical analysis; and determining the subject as having depression, anxiety, mood disorders, or neuropsychiatric symptoms or an increased risk of depression, anxiety, mood disorders, or neuropsychiatric symptoms when the concentration levels or ratios of the one or more biomarker metabolites or gut microbiome-related biomarker metabolites in the sample from the subject are different from (greater than or less than) the concentration levels or ratios of the one or more biomarker metabolites or gut microbiome-related biomarker metabolites in a control sample.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows the bile acid (BA) synthesis and cholesterol clearance pathway. Regulation of BA synthesis by feedback mechanism and BA transport through enterohepatic circulation. In the liver the bile acids (CDCA, DCA, LCA, CA) activate FXR that inhibits (via a repressor SHP, not shown here) the rate-limiting enzyme CYP7A1. The BAs via FXR/SHP also inhibit the influx transporter NTCP; induce BSEP and canalicular BA secretion. In the intestine, BAs, via FXR, inhibit the uptake transporter ASBT, decreasing absorption and increasing basolateral secretion into portal circulation by inducing OSTα and β. BA-activated FXR in the intestine also exerts inhibition on CYP7A1 in the liver via the FGF19 pathway. At the basolateral membrane of hepatocytes, transporters OSTα and β, and also MRP3 and MRP4, secrete BAs into the systemic circulation. Abbreviations: ASBT: Apical Sodium-dependent Bile acid Transporters; BA: Bile Acid; BSEP: Bile Salt Export Pump; CA: Cholic Acid; CDCA: Chenodeoxycholic Acid; DCA: Deoxycholic Acid: FXR: Farnesoid X Receptor; GCA: Glycocholic Acid; GCDCA: Glycochenodeoxycholic Acid; GLCA: Glycolithocholic Acid; G/T: ratio between glycine and taurine conjugated bile acids; HCA: Hydroxycitric Acid; HDCA: Hyodeoxycholic Acid; LCA: Lithocholic Acid; MCA: Monocarboxylic Acid; NTCP: Sodium/Taurocholate Co-transporting Polypeptide; SHP: Small heterodimer partner; TCA: Taurocholic Acid; TCDCA: Taurochenodeoxycholic Acid; UDCA: Ursodeoxycholic Acid.

FIG. 2 shows a heat map of Holm-corrected Spearman rank correlations between baseline clinical symptoms of depression in the Emory PREDICT Cohort. Abbreviations: HRSA-PSY: Psychic anxiety subscore of the Hamilton Anxiety Rating Scale; HRSA-SOM: Somatic anxiety subscore of the Hamilton Anxiety Rating Scale; HRSA-Total: 14-item Hamilton Anxiety Rating Scale; HRSD₁₇: 17-item Hamilton Depression Rating Scale; PReDICT: Predictors of Remission in Depression to Individual and Combined Treatments.

FIG. 3: shows correlations between baseline bile acids and depression and anxiety scores, and differences in baseline BA profiles between several participant groups. On the left: Heat map of partial Spearman rank correlations between baseline BAs and scores on the HRSD₁₇ and Hamilton Anxiety Rating Scale and subscales, after accounting for age, sex, and body mass index. On the right: Heat map of differences in baseline BA profiles in severe vs. non-severe depressed, high vs. low anxiety and treatment-failure vs. remitter groups. T-values were used for visualization purposes and the Wilcoxon Ranked Sum Test were used to test the significance of differences. Abbreviations: BA: Bile Acid; CA: Cholic Acid; CDCA: Chenodeoxycholic Acid; DCA: Deoxycholic Acid; GCA: Glycocholic Acid; GCDCA: Glycochenodeoxycholic Acid; GDCA: Glycodeoxycholic Acid; GHCA: Glycohyocholic Acid; GHDCA: Glycohyodeoxycholic Acid; GLCA: Glycolithocholic Acid; GLCA_3 S: Glycolithocholic Acid 3-Sulfate; GUDCA: Glycoursodeoxycholic Acid; HCA: Hydroxycitric Acid; HDCA: Hyodeoxycholic Acid; HRSA-PSY: Psychic anxiety subscore of the Hamilton Anxiety Rating Scale; HRSA-SOM: Somatic anxiety subscore of the Hamilton Anxiety Rating Scale; HRSA-Total: 14-item Hamilton Anxiety Rating Scale; HRSD₁₇: 17-item Hamilton Depression Rating Scale; LCA: Lithocholic Acid; LCA_3 S: Lithocholic Acid 3-sulfate; MCA: Monocarboxylic Acid; TCA: Taurocholic Acid; TCDCA: Taurochenodeoxycholic Acid; TDCA: Taurodeoxycholic Acid; THCA: Tetrahydrocannabinolic Acid; THDCA: Taurohyodeoxycholic Acid; TUDCA: Tauroursodeoxycholic Acid; UCA: Ursocholic Acid; UDCA: Ursodeoxycholic Acid.

FIG. 4 shows scatter plots of HRSD₁₇ scores by HRSA-total interaction for selected bile acids and ratios. Abbreviations: Anx: Anxiety; CA: Cholic Acid; CDCA: Chenodeoxycholic Acid; Dep: Depression; HRSA-Total: 14-item Hamilton Anxiety Rating Scale; HRSD₁₇: 17-item Hamilton Depression Rating Scale; LCA: Lithocholic Acid.

FIG. 5A-B show ratios of BAs reflective of liver and gut microbiome enzymatic activities in depressed patients. Three types of ratios (pairwise or composite) were calculated to inform about possible enzymatic activity changes in depressed participants. These ratios reflect one of the following: (1) Shift in BA metabolism from primary to alternative pathway. (2) Changes in gut microbiome correlated with production of secondary BAs. (3) Changes in glycine and taurine conjugation of BAs. FIG. 5A shows a heat map of partial Spearman rank correlations between BA ratios/summations and scores on the HRSD₁₇ and Hamilton Anxiety scale and subscales, after accounting for age, sex, and body mass index. FIG. 5B shows a heat map of differences in ratios/summations in severe vs. non-severe depressed, high vs. low anxious and treatment-failure vs. remitter groups. Abbreviations: BA: Bile Acid; CA: Cholic Acid; CDCA: Chenodeoxycholic Acid; DCA: Deoxycholic Acid; GCA: Glycocholic Acid; GCDCA: Glycochenodeoxycholic Acid; GDCA: Glycodeoxycholic Acid; GHCA: Glycohyocholic Acid; GHDCA: Glycohyodeoxycholic Acid; GLCA: Glycolithocholic Acid; GUDCA: Glycoursodeoxycholic Acid; HCA: Hydroxycitric Acid; HDCA: Hyodeoxycholic Acid; HRSA-PSY: Psychic anxiety subscore of the Hamilton Anxiety Rating Scale; HRSA-SOM: Somatic anxiety subscore of the Hamilton Anxiety Rating Scale; HRSA-Total: 14-item Hamilton Anxiety Rating Scale; HRSD₁₇: 17-item Hamilton Depression Rating Scale (HRSD₁₇); LCA: Lithocholic Acid; MCA: Monocarboxylic Acid; TCA: Taurocholic Acid; TCDCA: Taurochenodeoxycholic Acid; TDCA: Taurodeoxycholic Acid; THCA: Tetrahydrocannabinolic Acid; THDCA: Taurohyodeoxycholic Acid; TUDCA: Tauroursodeoxycholic Acid; UDCA: Ursodeoxycholic Acid.

FIG. 6 shows scatter plots of baseline concentration of selected bile acids and bile acid ratios in treatment failure versus remission groups. Abbreviations: CA: Cholic Acid; CDCA: Chenodeoxycholic Acid; GHCA: Glycohyocholic Acid; GLCA: Glycolithocholic Acid; LCA: Lithocholic Acid; MCA: Monocarboxylic Acid.

FIG. 7 shows a summary of findings. Abbreviations: CDCA: Chenodeoxycholic Acid; GHCA: Glycohyocholic Acid; GLCA: Glycolithocholic Acid; GLCA_3 S: Glycolithocholic Acid 3 Sulfate; HCA: Hydroxycitric Acid; HDCA: Hyodeoxycholic Acid; LCA: Lithocholic Acid; LCA_3 S: Lithocholic Acid 3-Sulfate; MCA: Monocarboxylic Acid; UDCA: Ursodeoxycholic Acid.

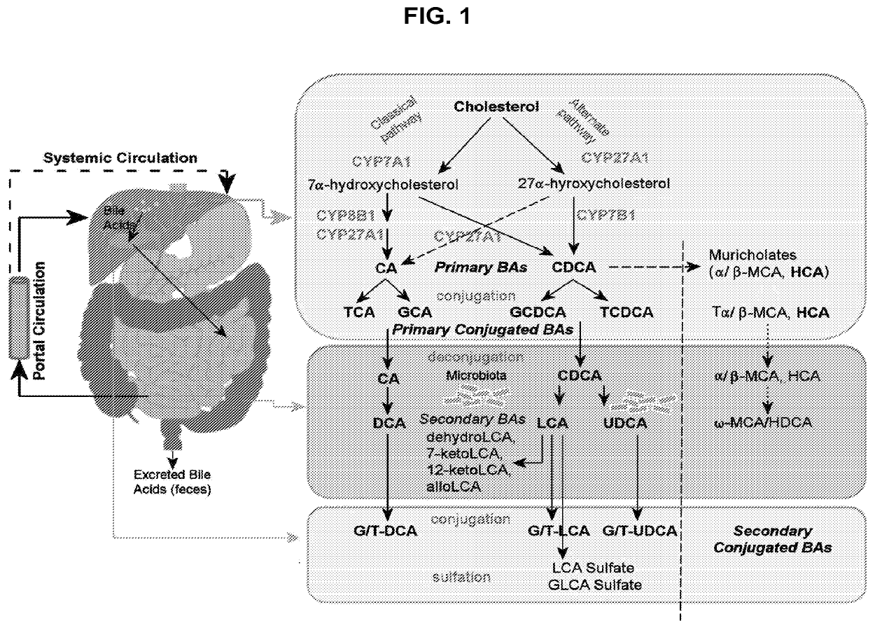

### DETAILED DESCRIPTION

Unless otherwise defined, all technical and scientific terms used herein have the same meaning as commonly understood by one of ordinary skill in the art. For example, any nomenclatures used in connection with, and techniques of, cell and tissue culture, molecular biology, immunology, microbiology, genetics, and protein and nucleic acid chemistry and hybridization described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

As used herein, the terms such as “include,” “including,” “contain,” “containing,” “having,” and the like mean “comprising.” The present disclosure also contemplates other embodiments “comprising,” “consisting of,” and “consisting essentially of,” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points.

As used herein, the terms “active ingredient” or “active pharmaceutical ingredient” refer to a pharmaceutical agent, active ingredient, compound, or substance, compositions, or mixtures thereof, that provide a pharmacological, often beneficial, effect.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “dose” denotes any form of an active ingredient formulation or composition, including cells, that contains an amount sufficient to initiate or produce a therapeutic effect with at least one or more administrations. “Formulation” and “composition” are used interchangeably herein.

As used herein, the term “prophylaxis” refers to preventing or reducing the progression of a disorder, either to a statistically significant degree or to a degree detectable by a person of ordinary skill in the art.

As used herein, the terms “effective amount” or “therapeutically effective amount,” refers to a substantially non-toxic, but sufficient amount of an action, agent, composition, or cell(s) being administered to a subject that will prevent, treat, or ameliorate to some extent one or more of the symptoms of the disease or condition being experienced or that the subject is susceptible to contracting. The result can be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human.

As used herein, a subject is “in need of treatment” if such subject would benefit biologically, medically, or in quality of life from such treatment. A subject in need of treatment does not necessarily present symptoms, particular in the case of preventative or prophylaxis treatments.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, “treatment” or “treating” refers to prophylaxis of, preventing, suppressing, repressing, reversing, alleviating, ameliorating, or inhibiting the progress of biological process including a disorder or disease, or completely eliminating a disease. A treatment may be either performed in an acute or chronic way. The term “treatment” also refers to reducing the severity of a disease or symptoms associated with such disease prior to affliction with the disease. “Repressing” or “ameliorating” a disease, disorder, or the symptoms thereof involves administering a cell, composition, or compound described herein to a subject after clinical appearance of such disease, disorder, or its symptoms. “Prophylaxis of” or “preventing” a disease, disorder, or the symptoms thereof involves administering a cell, composition, or compound described herein to a subject prior to onset of the disease, disorder, or the symptoms thereof. “Suppressing” a disease or disorder involves administering a cell, composition, or compound described herein to a subject after induction of the disease or disorder thereof but before its clinical appearance or symptoms thereof have manifest.

As used herein, “treatment,” “therapy,” or “therapy regimen” also refer to the clinical intervention made in response to a disease, disorder, or physiological condition (e.g., a depressive disorder) manifested by a patient or to which a patient may be susceptible. The aim of treatment includes the alleviation or prevention of symptoms, slowing or stopping the progression or worsening of a disease, disorder, or condition and/or the remission of the disease, disorder, or condition (e.g., a depressive disorder). As used herein, the terms “prevent,” “preventing,” “prevention,” “prophylactic treatment” and the like refer to reducing the probability of developing a disease, disorder or condition in a subject, who does not have, but is at risk of or susceptible to developing a disease, disorder or condition. The term “effective amount” or “therapeutically effective amount” refers to an amount sufficient to effect beneficial or desirable biological and/or clinical results.

As used herein, the term “administering” an agent, such as a therapeutic entity to treat a depressive disorder to an animal or cell, is intended to refer to dispensing, delivering, or applying the substance to the intended target. In terms of the therapeutic agent, the term “administering” is intended to refer to contacting or dispensing, delivering or applying the therapeutic agent to a subject by any suitable route for delivery of the therapeutic agent to the desired location in the animal, including delivery by either the parenteral or oral route, intramuscular injection, subcutaneous/intradermal injection, intravenous injection, intrathecal administration, buccal administration, transdermal delivery, topical administration, and administration by the intranasal or respiratory tract route.

As used herein, the term “biomarker” refers to a naturally occurring biological molecule present in a subject at varying concentrations useful in predicting the risk or incidence of a disease or a condition, such as a depressive disorder. For example, the biomarker can be a protein present in higher or lower amounts in a subject at risk for a depressive disorder. The biomarker can include nucleic acids, ribonucleic acids, metabolite, protein, fatty acid, lipid, polypeptide or the like that is used as an indicator or marker for a depressive disorder in the subject. Biomarkers may reflect a variety of disease characteristics, including the level of exposure to an environmental or genetic trigger, an element of the disease process itself, and intermediate stage between exposure and disease onset, or an independent factor associated with the disease state, but not causative of pathogenesis. Biomarkers may be used to determine the status of a subject or the effectiveness of a treatment. Biomarker combinations with the most diagnostic utility have both high sensitivity and specificity. In practice, biomarkers and/or specific combinations of biomarkers having both high sensitivity and specificity are not obvious. Evaluation, assessment, and combination of specific biomarkers for diagnosis provide an improved approach to disease treatment.

The term “biological sample” as used herein includes, but is not limited to, a sample containing tissues, cells, including peripheral cells in human blood, and/or biological fluids isolated from a subject. Examples of biological samples include, but are not limited to, tissues, cells, biopsies, muscle, interstitial fluid, sweat, saliva, urine, tears, synovial fluid, bone marrow, cerebrospinal fluid, nasal secretions, sputum, mucus, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In one embodiment, the biological sample comprises a blood sample. A biological sample may be obtained directly from a subject (e.g., by blood or tissue sampling) or from a third party (e.g., received from an intermediary, such as a healthcare provider or lab technician).

As used herein, “depression” refers to a mood disorder that causes a persistent feeling of sadness and loss of interest. As used herein, “depression” includes subclinical characteristics associated with depression such as sadness, loss of interest in activities, loss of appetite, anhedonia, insomnia, changes in sleep, difficulty falling asleep, waking during the night, restless sleep, waking too early, sleeping too much, low energy level, lack of concentration, diminished or altered daily behavior, low self-esteem, suicidal thoughts, anxiety coupled with depression, or combinations thereof. Included in this definition is Major Depression Disorder (MDD), core depression (CD+), anxious depression (ANX+), neurovegetative symptoms of melancholia (NVSM+), subclinical characteristics associated with depression, or a neuropsychiatric symptom associated with a neurological disease or cognitive impairment. As used herein “major depressive disorder” refers to a mental health disorder characterized by persistent feeling of sadness or loss of interest that characterizes major depression can lead to a range of behavioral and physical symptoms causing significant impairment in daily life. These may include depressed mood, loss of interest in activities, changes in sleep, appetite, energy level, concentration, daily behavior, self-esteem, or suicide ideation. In one aspect, depression comprises treatment resistant depression. Anxiety is often noted in patients who are depressed hence we refer to depression and anxiety as comorbidities.

Treatments for depressive disorders may include any of those currently available treatments and can be readily determined by one skilled in the art. Such treatments may include antidepressants, including but not limited to, tranylcypromine, phenelzine, selegiline, isocarboxazid, amitriptyline, clomipramine, desipramine, doxepin, imipramine, nortryptyline, amoxapine, protriptyline, trimipramine, bupropion, nefazodone, venlafaxine, mirtazapine, duloxetine, fluoxetine, fluvoxamine, paroxetine, sertraline, citalopram, or escitalopram. In one aspect, the antidepressant is one or more selective serotonin reuptake inhibitors (SSRI). In another aspect, the antidepressant comprises an SSRI selected from escitalopram, citalopram, fluoxetine, sertraline, paroxetine, fluvoxamine, vilazodone, vortioxetine, or duloxetine. In another aspect, the antidepressant comprises an SSRI comprising escitalopram or citalopram. In another aspect, the antidepressant comprises an NMDA receptor antagonist, such as ketamine.

As used herein, “anxiety” refers to anxiety disorders including generalized anxiety disorder, panic attacks, obsessive-compulsive disorders, phobias, and post-traumatic stress disorders. Symptoms include feelings of apprehension or dread or impending doom, feeling tense or jumpy, restlessness or irritability, difficulty controlling feelings of worry, anticipating the worst and being watchful for signs of danger, difficulty concentrating or mind going blank, and physical symptoms including heart palpitations, pounding or racing heart, shortness of breath, sweating, tremors or shaking, muscle tension, headaches, fatigue, insomnia, upset stomach, frequent urination, or diarrhea.

As used herein, the terms “neurological diseases” or “neurological disorders” are used interchangeably and refer to a host of undesirable conditions affecting neurons in the brain of a subject. Representative examples of such conditions include, without limitation, Alzheimer's disease, Parkinson's disease, Huntington's disease, Pick's disease, Kufs disease, Lewy body disease, neurofibrillary tangles, Rosenthal fibers, Mallory's hyaline, senile dementia, myasthenia gravis, Gilles de la Tourette's syndrome, multiple sclerosis (MS), amyotrophic lateral sclerosis (ALS), progressive supranuclear palsy (PSP), epilepsy, Creutzfeldt-Jakob disease, deafness-dystonia syndrome, Leigh syndrome, Leber hereditary optic neuropathy (LHON), Parkinsonism, dystonia, motor neuron disease, neuropathy-ataxia and retinitis pigmentosa (NARP), maternal inherited Leigh syndrome (MILS), Friedreich ataxia, hereditary spastic paraplegia, Mohr-Tranebjaerg syndrome, Wilson disease, sporadic Alzheimer's disease, sporadic amyotrophic lateral sclerosis, sporadic Parkinson's disease, autonomic function disorders, hypertension, sleep disorders, neuropsychiatric disorders, depression, autism, schizophrenia, schizoaffective disorder, Korsakoff's psychosis, mania, anxiety disorders, phobic disorder, learning or memory disorders, amnesia or age-related memory loss, attention deficit disorder, dysthymic disorder, major depressive disorder, obsessive-compulsive disorder, psychoactive substance use disorders, panic disorder, bipolar affective disorder, severe bipolar affective (mood) disorder (BP-1), migraines, hyperactivity and movement disorders

As used herein, the term “neuropsychiatric symptom” refers to non-cognitive symptoms that are commonly occur during the course of Alzheimer's disease or dementia. These neuropsychiatric symptoms (NPS) include depression, anxiety, aggression, agitation, delusions, hallucinations, apathy, or disinhibition. Many CNS disease include neuropsychiatric symptoms such sleep disruption, depression anxiety all of these disorders might have links to gut microbiome changes and changes in bile acids and can benefit from approaches for the methods and treatments described herein.

As used herein, the term “movement disorder” includes neurological diseases or disorders that involve the motor and movement systems, resulting in a range of abnormalities that affect the speed, quality, and ease of movement. Movement disorders are often caused by or related to abnormalities in brain structure and/or function. Movement disorders include, but are not limited to (i) tremors: including, but not limited to, the tremor associated with Parkinson's Disease, physiologic tremor, benign familial tremor, cerebellar tremor, rubral tremor, toxic tremor, metabolic tremor, and senile tremor; (ii) chorea, including, but not limited to, chorea associated with Huntington's Disease, Wilson's Disease, ataxia telangiectasia, infection, drug ingestion, or metabolic, vascular or endocrine etiology (e.g., chorea gravidarum or thyrotoxicosis); (iii) ballism (defined herein as abruptly beginning, repetitive, wide, flinging movements affecting predominantly the proximal limb and girdle muscles); (iv) athetosis (defined herein as relatively slow, twisting, writhing, snake-like movements and postures involving the trunk, neck, face and extremities); (v) dystonia (defined herein as a movement disorder consisting of twisting, turning tonic skeletal muscle contractions, most, but not all of which are initiated distally); (vi) paroxysmal choreoathetosis and tonic spasm; (vii) tics (defined herein as sudden, behaviorally related, irregular, stereotyped, repetitive movements of variable complexity); (viii) tardive dyskinesia; (ix) akathisia, (x) muscle rigidity, defined herein as resistance of a muscle to stretch; (xi) postural instability; (xii) bradykinesia; (xiii) difficulty in initiating movements; (xiv) muscle cramps; (xv) dyskinesias and (xvi) myoclonus. In some embodiments, the neurological disorder comprises dystonia.

As used herein the terms “bile acid(s)” or “bile acid derivative(s),” (also abbreviated as “BA”) refer to any of the known bile acids and derivatives thereof, including primary and secondary bile acids, as well as any derivative of any of the known bile acids or derivatives thereof, including derivatives of a primary bile acid, a secondary bile acid, a conjugated primary acid, or a conjugated secondary bile acid. Glycine and taurine conjugation are well known but there are many additional conjugations that are just beginning to emerge. Ratios and summation of bile acids as well as conjugation all might impact key biological functions related to neuropsychiatric diseases and could inform and enable patient stratification and treatment approaches for subgroups of patients. As one of skill in the art would recognize based on the present disclosure, there are many known bile acids, including but not limited to, primary bile acids cholic acid (CA) and chenodeoxycholic acid (CDCA) and their conjugated glycine and taurine forms; secondary bile acids such as deoxycholic acid, taurocholic acid, glycocholic acid, glycodeoxycholic acid, taurodeoxycholic acid, ursodeoxycholic acid and chenodeoxycholic acid, and any derivatives thereof. See e.g., Table 2, below. Each of these compounds can also be functionalized and substituted to encompass a class of compounds, which includes among other things, oxidized and reduced analogs, alkylated and acylated analogs, cyclized or bis-cyclized analogs, and analogs having a shorter or longer side chain. All of these bile acids and bile acid derivatives are included in the terms “bile acid(s)” and “bile acid derivative(s).”

As used herein the phrase “bile acid modulating agent(s),” refers to an agent (e.g., small molecule compound, biologic molecule, aptamer, and any combinations thereof) capable of modulating the synthesis and/or processing of a bile acid or a bile acid derivative, molecules that are structurally similar to bile acids, protective bile acids or their analogs (e.g., chenodeoxycholic acid (CDCA), ursodeoxycholic acid (UDCA), tauroursodeoxycholic acid (TUDCA) and glycoursodeoxycholic acid (GUDCA)). Modulating can include both increasing activity (e.g., activation, stimulation, etc.) and decreasing (e.g., attenuation, inhibition, etc.) activity of the particular target or targets of the bile acid modulating agent, such that bile acid synthesis and/or processing is ultimately modulated. For example, a bile acid modulating agent can include agents capable of reducing the levels of secondary bile acids as compared to primary bile acids.

Human enzymes producing primary bile acids, such as CA and CDCA can be modulated or supplemented to increase their concentrations. Drugs can be designed that mimic their structure and activity and that modulate their targets. Gut bacterial enzymes that produce toxic secondary bile acids can be targeted for downregulation or inhibition. Gut microbiome organisms can be supplemented or replaced with genetically modified strains that do not produce toxic compounds in subjects with mood disorders. Toxic bile acids can be sequestered or trapped using clathrins or other cage molecules. Signaling pathways affected by bile acids can be modulated or targeted for upregulation or inhibition. Drugs currently developed for other diseases that target bile acids are useful for modulating bile acid metabolism. For example, bile acid sequestrants such as cholestyramine (Questran®, Prevalite®), colestipol (Colestid®), and colesevelam (Welchol®), can be used to capture bile acids. Bile acid analogs, such as 12-monoketocholic acid, 7-monoketocholic acid, 7,12-diketocholic acid, 3,7,12-triketocholic acid, 12-monoketdeoxyocholic acid, are useful nontoxic analogs. Further, steroidal or nonsteroidal agonists of bile acid receptors, agonists of TGR5 G-protein-coupled receptors, agonist of farnesoid X receptor (FXR), and agonists or activators of cholesterol 7α-hydroxylase enzyme (encoded by CYP7A1) are useful for modulating bile acid metabolism.

Described herein are the results of a study that profiled circulating metabolites in drug-free MDD outpatients with varying severity of symptoms of depression and anxiety. These metabolites have been previously reported to derive either from bacterial metabolism of dietary substrates, modification of host molecules, or directly from bacteria and participate in a large number of host functions.

In some embodiments, gut bacteria-enriched metabolites were profiled in serum samples from 208 untreated patients with moderate-to-severe major depressive disorder (MDD) and assessed their relationship with severity of symptoms of depression and anxiety in the overall study sample as well as separately by sex. Several long chain free fatty acids including the polyunsaturated fatty acids like arachidonic acid, eicosapentaenoic acid, and docosahexaenoic acid, were depleted in patients with more severe symptoms of depression and anxiety and this pattern was more pronounced in males than females. In contrast, the short chain fatty acids trended to be higher in the more severely anxious patients. Gut bacteria-derived secondary bile acids and the ratios of the secondary bile acids to their primary precursors were significantly elevated in the severely anxious patients, both males and females. Significant depletions were observed in citric acid cycle intermediates, citrate and cis-aconitate while several amino acids and sulfates were higher in the highly anxious patients. Overall, a gut microbial dysbiosis was clearly evident in the MDD patients that seemed to affect males and females in different ways. The impact was more strongly correlated to the symptoms of anxiety than depression and suggests systems level metabolic alterations related to the gut microbiome that underscores a pathophysiology of inflammation, energy homeostasis and immune system perturbations. One embodiment provides for methods of diagnosing, screening, predicting and/or detecting a depressive disorder in a subject, the methods comprising, consisting of, or consisting essentially of obtaining a biological sample from the subject, determining the concentration level of a gut microbiome-related biomarker metabolites as provided herein in the subject, comparing the levels of the one or more free fatty acid biomarker as provided herein against a control sample, diagnosing, screening, predicting, and/or detecting the depressive disorder in the subject if the one or more gut microbiome-related biomarker metabolites is different from (greater or less than) that of the control sample.

Another embodiment provides methods of diagnosing, screening, predicting, and/or detecting a neurological disease or disorder in a subject, the methods comprising, consisting of, or consisting essentially of obtaining a biological sample from the subject, determining the concentration level of one or more gut microbiome-related biomarker metabolites as provided herein in the subject, comparing the levels of the one or more free fatty acid biomarker metabolites as provided herein against a control sample, diagnosing, screening, predicting, and/or detecting the depressive disorder in the subject if the one or more gut microbiome-related biomarker metabolites is different from (greater or less than) that of the control sample. The cholesterol metabolic pathway and clearance pathway can be analyzed using both genetic and metabolic profiling to determine whether there are inborn errors in metabolism that lead to defects in bile acid metabolism, anabolism, or catabolism. Specific treatments targeting the genetic or metabolic defects can be tailored for each subject or subgroup of subjects with common defects.

Several therapeutic approaches can be used to ameliorate defects or errors in cholesterol and bile acid metabolism, anabolism, catabolism and clearance including modulating human enzymes to increase production of primary bile acids; modulating bacterial enzymes to reduce or inhibit the production of toxic secondary bile acids, supplementing primary bile acids, supplementing protective bile acids, such as chenodeoxycholic acid (CDCA), ursodeoxycholic acid (UDCA), tauroursodeoxycholic acid (TUDCA) and glycoursodeoxycholic acid (GUDCA), administering therapeutics to target enzymes or signaling pathways in primary or secondary bile acid metabolism, supplementing or replacing aberrant gut microbiome organisms with natural or genetically modified organisms to reduce toxic secondary bile acids, administering compounds to chemically trap toxic bile acids, or administering agents that modify or up/down-regulate signaling pathways or feedback mechanisms in bile acid metabolism. Specific drugs designed for other liver or bile acid disorders are particularly useful as therapeutics.

In some embodiments, the gut microbiome-related biomarker metabolites comprise one or more short chain fatty acids and/or one or more medium chain fatty acids, and/or one or more long chain fatty acids. In one embodiment, the one or more free fatty acid biomarker metabolites are selected from the group provided in the Example section.

In another embodiments, the gut microbiome-related biomarker metabolites comprise a primary bile acid. In one embodiment, the primary bile acid comprises CDCA. In another embodiment, the primary bile acid comprises CA.

Another embodiment provides for methods of treating a depressive disorder in a subject, the methods comprising, consisting of, or consisting essentially of obtaining a biological sample from the subject, determining the concentration level of one or more gut microbiome-related biomarker metabolites as provided herein in the subject, comparing the levels of the one or more gut microbiome-related biomarker metabolites as provided herein against a control sample, treating the depressive disorder in the subject if the one or more gut microbiome-related biomarker metabolites is different from (greater or less than) that of the control sample.

Another embodiment provides methods of treating a neurological disease or disorder in a subject, the methods comprising, consisting of, or consisting essentially of obtaining a biological sample from the subject, determining the concentration level of one or more gut microbiome-related biomarker metabolites as provided herein in the subject, comparing the levels of the one or more gut microbiome-related biomarker metabolites as provided herein against a control sample, and treating the depressive disorder in the subject if the one or more gut microbiome-related biomarker metabolites is different from (greater or less than) that of the control sample.

In some embodiments, the gut microbiome-related biomarker metabolites comprise one or more short chain fatty acids and/or one or more medium chain fatty acids, and/or one or more long chain fatty acids. In one embodiment, the one or more free fatty acid biomarker metabolites are selected from the group provided in the Example section.

In another embodiments, the gut microbiome-related biomarker metabolites comprise a primary bile acid. In one embodiment, the primary bile acid comprises CDCA. In another embodiment, the primary bile acid comprises CA.

In one embodiment, the method further comprises administering to the subject a therapeutically effective amount of one or more short and/or medium chain fatty acids and/or long-chain fatty acids, and/or any pharmaceutically acceptable derivatives, esters, salts, solvates, hydrates, analogs, prodrugs thereof and/or a therapeutic agent capable of modulating (either increasing or decreasing) the levels of one or more short and/or medium chain fatty acids, and/or any pharmaceutically acceptable derivatives, esters, salts, solvates, hydrates, analogs, prodrugs thereof and/or a therapeutic agent capable of modulating (either increasing or decreasing) the levels of one or more one or more short and/or medium chain fatty acids and/or long-chain fatty acid, activating their production endogenously, and/or modulating synthesis breakdown, and/or incorporating into a lipid carrier. Medium chain fatty acids mimic structural analogs of bile acids and modulators of targets and links to lipid metabolism.

In another embodiment, the method further comprises administering to the subject a therapeutically effective amount of one or more primary bile acids and/or any pharmaceutically acceptable derivatives, esters, salts, solvates, hydrates, analogs, prodrugs thereof and/or a therapeutic agent capable of modulating (either increasing or decreasing) the levels of one or more primary bile acids, activating their production endogenously, and/or decreasing their breakdown.

In another embodiment, the primary bile acid comprises CDCA. In another embodiment, the primary bile acid comprises CA.

Another embodiment described herein are compositions that may comprise one or more therapeutic agents, metabolites, and combinations thereof as described herein and an appropriate carrier, excipient, or diluent. The exact nature of the carrier, excipient or diluent will depend upon the desired use for the composition and may range from being suitable or acceptable for veterinary uses to being suitable or acceptable for human use. The composition may optionally include one or more additional compounds.

When used to treat or prevent such diseases, such as neurological disorders or depression, anxiety, mood disorders or neuropsychiatric disorders, the compounds described herein may be administered singly, as mixtures of one or more compounds or in mixture or combination with other agents useful for treating such diseases and/or the symptoms associated with such diseases. The compounds may also be administered in mixture or in combination with agents useful to treat other disorders or maladies, such as antidepressive agents, including but not limited to, ketamine, tranylcypromine, phenelzine, selegiline, isocarboxazid, amitriptyline, clomipramine, desipramine, doxepin, imipramine, nortryptyline, amoxapine, protriptyline, trimipramine, bupropion, nefazodone, venlafaxine, mirtazapine, duloxetine, fluoxetine, fluvoxamine, paroxetine, sertraline, citalopram, or escitalopram. In one aspect, the antidepressant is one or more selective serotonin reuptake inhibitors (SSRI). In another aspect, the antidepressant comprises an SSRI selected from escitalopram, citalopram, fluoxetine, sertraline, paroxetine, fluvoxamine, vilazodone, vortioxetine, ketamine, or duloxetine. The compound may include agents which prevents or reduces symptoms of the neurological disorder, including, but not limited to, anticholinergics, such as trihexyphenidyl (Artane®), benztropine (Cogentin®), ethopropazine (Parsitan®); benzodiazepines, such as diazepam (Valium®), clonazepam (Klonopin®), lorazepam (Ativan®); baclofen (Lioresal®), dopaminergic agents such as levodopa (Sinemet®) and bromocriptine (Parlodel®); tetrabenazine (Xenazine®), dopamine-depleting agents, ritonavir, lopinavir, and the like.

Pharmaceutical compositions comprising the compound(s) may be manufactured by means of conventional mixing, dissolving, granulating, dragee-making levigating, emulsifying, encapsulating, entrapping or lyophilization processes. The compositions may be formulated in conventional manner using one or more physiologically acceptable carriers, diluents, excipients, or auxiliaries which facilitate processing of the compounds into preparations which can be used pharmaceutically.

The compounds may be formulated in the pharmaceutical composition per se, or in the form of a hydrate, solvate, N-oxide or pharmaceutically acceptable salt, as previously described. Typically, such salts are more soluble in aqueous solutions than the corresponding free acids and bases, but salts having lower solubility than the corresponding free acids and bases may also be formed.

Pharmaceutical compositions may take a form suitable for virtually any mode of administration, including, for example, topical, ocular, oral, buccal, systemic, nasal, injection, transdermal, rectal, vaginal, etc., or a form suitable for administration by inhalation or insufflation.

For topical administration, the compound(s) may be formulated as solutions, gels, ointments, creams, suspensions, etc. as are well-known in the art. Systemic formulations include those designed for administration by injection, e.g., subcutaneous, intravenous, intramuscular, intrathecal, or intraperitoneal injection, as well as those designed for transdermal, transmucosal oral or pulmonary administration.

Useful injectable preparations include sterile suspensions, solutions, or emulsions of the active compound(s) in aqueous or oily vehicles. The compositions may also contain formulating agents, such as suspending, stabilizing and/or dispersing agent. The formulations for injection may be presented in unit dosage form, e.g., in ampules or in multidose containers, and may contain added preservatives. Alternatively, the injectable formulation may be provided in powder form for reconstitution with a suitable vehicle, including but not limited to sterile pyrogen free water, buffer, dextrose solution, etc., before use. To this end, the active compound(s) may be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art with, for example, sugars, films, or enteric coatings.

Liquid preparations for oral administration may take the form of, for example, elixirs, solutions, syrups, or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol, Cremophore™ or fractionated vegetable oils); and preservatives (e.g., methyl or propyl-p-hydroxybenzoates or sorbic acid). The preparations may also contain buffer salts, preservatives, flavoring, coloring, and sweetening agents as appropriate.

Preparations for oral administration may be suitably formulated to give controlled release of the compound, as is well known. For buccal administration, the compositions may take the form of tablets or lozenges formulated in conventional manner. For rectal and vaginal routes of administration, the compound(s) may be formulated as solutions (for retention enemas) suppositories or ointments containing conventional suppository bases such as cocoa butter or other glycerides.

For nasal administration or administration by inhalation or insufflation, the compound(s) can be conveniently delivered in the form of an aerosol spray from pressurized packs or a nebulizer with the use of a suitable propellant, e.g., dichlorodifluoromethane, trichlorofluoromethane, dichlorotetrafluoroethane, fluorocarbons, carbon dioxide or other suitable gas. In the case of a pressurized aerosol, the dosage unit may be determined by providing a valve to deliver a metered amount. Capsules and cartridges for use in an inhaler or insufflator (for example capsules and cartridges comprised of gelatin) may be formulated containing a powder mix of the compound and a suitable powder base such as lactose or starch.

For ocular administration, the compound(s) may be formulated as a solution, emulsion, suspension, etc. suitable for administration to the eye. A variety of vehicles suitable for administering compounds to the eye are known in the art.

For prolonged delivery, the compound(s) can be formulated as a depot preparation for administration by implantation or intramuscular injection. The compound(s) may be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch which slowly releases the compound(s) for percutaneous absorption may be used. To this end, permeation enhancers may be used to facilitate transdermal penetration of the compound(s).

Alternatively, other pharmaceutical delivery systems may be employed. Liposomes and emulsions are well-known examples of delivery vehicles that may be used to deliver compound(s). Certain organic solvents such as dimethyl sulfoxide (DMSO) may also be employed, although usually at the cost of greater toxicity.

The pharmaceutical compositions may, if desired, be presented in a pack or dispenser device which may contain one or more dosage forms containing the compound(s). The pack may, for example, comprise metal or plastic foil, such as a blister pack. The pack or dispenser device may be accompanied by instructions for administration.

The compound(s) described herein, or compositions thereof, will generally be used in an amount effective to achieve the intended result, for example in an amount effective to treat or prevent the particular disease being treated. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. Therapeutic benefit also generally includes halting or slowing the progression of the disease, regardless of whether improvement is realized.

The amount of compound(s) administered will depend upon a variety of factors, including, for example, the particular indication being treated, the mode of administration, whether the desired benefit is prophylactic or therapeutic, the severity of the indication being treated and the age and weight of the patient, the bioavailability of the particular compound(s) the conversation rate and efficiency into active drug compound under the selected route of administration, etc.

Determination of an effective dosage of compound(s) for a particular use and mode of administration is well within the capabilities of those skilled in the art. Effective dosages may be estimated initially from in vitro activity and metabolism assays. For example, an initial dosage of compound for use in animals may be formulated to achieve a circulating blood or serum concentration of the metabolite active compound that is at or above an IC₅₀ of the particular compound as measured in as in vitro assay. Calculating dosages to achieve such circulating blood or serum concentrations considering the bioavailability of the particular compound via the desired route of administration is well within the capabilities of skilled artisans. Initial dosages of compound can also be estimated from in vivo data, such as animal models. Animal models useful for testing the efficacy of the active metabolites to treat or prevent the various diseases described above are well-known in the art. Animal models suitable for testing the bioavailability and/or metabolism of compounds into active metabolites are also well-known. Ordinarily skilled artisans can routinely adapt such information to determine dosages of particular compounds suitable for human administration.

Dosage amounts will typically be in the range of from about 0.0001 mg/kg/day. 0.001 mg/kg/day or 0.01 mg/kg/day to about 100 mg/kg/day, but may be higher or lower, depending upon, among other factors, the activity of the active compound, the bioavailability of the compound, its metabolism kinetics and other pharmacokinetic properties, the mode of administration and various other factors, discussed above. Dosage amount and interval may be adjusted individually to provide plasma levels of the compound(s) and/or active metabolite compound(s) which are sufficient to maintain therapeutic or prophylactic effect. For example, the compounds may be administered once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of compound(s) and/or active metabolite compound(s) may not be related to plasma concentration. Skilled artisans will be able to optimize effective dosages without undue experimentation.

One embodiment described herein is a method for the classification and treatment of depression, anxiety, mood disorders, or neuropsychiatric symptoms in a subject based on the subject's metabolic profile and genetic screening, the method comprising one or more of the following: identifying and stratifying subjects afflicted with a depression, anxiety, mood disorders, or neuropsychiatric symptoms to subgroups based on their metabolic profiles, biomarker metabolites and ratios of biomarker metabolites that define unique metabolic conditions related to aberrations in cholesterol metabolism or bile acid anabolism, catabolism homeostasis, or clearance and common identity among subgroups of subjects; evaluating the trajectory of disease within each stratified subgroup of subjects and their response to a therapeutic treatment; identifying defects in bile acid transport and/or biosynthesis of biomarker metabolites within a metabolic pathway or across metabolic pathways using ratios of biomarker metabolites to inform about changes in enzyme activities or transporters; and identifying genetic bases of bile acid metabolic profile characteristics or defects (SNPs/genetic variants in key enzymes and transporters) using metabolite genome-wide association study (mGWAS) analysis. In one aspect, the method further comprises one or more of the following: using combined metabotype and genotype data to better stratify subjects with depression, anxiety, mood disorders, or neuropsychiatric symptoms and to inform about mechanisms and treatment selection; suggesting a therapeutic approach to correct metabolic defects in metabolic profile in stratified subgroups of subjects; and comparing and contrasting metabolic defects noted in inborn errors of metabolism that have depression, anxiety, mood disorders, or neuropsychiatric symptoms and using knowledge gained in treatment of inborn errors of metabolism to inform treatment for depression, anxiety, mood disorders, or neuropsychiatric symptoms. In another aspect, the method further comprises performing imaging analysis on the subject and linking peripheral changes with brain changes. In another aspect, the method further comprises administering to the subjects an effective amount of a therapy to prevent and/or treat the depression, anxiety, mood disorders, or neuropsychiatric symptoms affected by one or more metabolic defects. In another aspect, the depression, anxiety, mood disorders, or neuropsychiatric symptoms are associated with neurological diseases or cognitive impairment, including dementia, vascular dementia, mixed dementia, early mild cognitive impairment (EMCI), late mild cognitive impairment (LMCI), Alzheimer's Disease, dementia with Lewy bodies, frontotemporal dementia, Creutzfeldt-Jakob disease, Parkinson's Disease, young-onset dementia, Korsakoff's syndrome, Huntington's disease, HIV-associated neurocognitive disorders, or other cognitive impairment disorders.

Another embodiment described herein is a method for stratifying and treating a subject having a depression, anxiety, mood disorders, or neuropsychiatric symptoms, or at risk of developing a neurological disorder, based on the subject's metabolic profile, the method comprising: analyzing a sample from a subject to determine concentration levels or ratios of one or more biomarker metabolites or gut microbiome-related biomarker metabolites related to bile acid anabolism, catabolism or homeostasis in the sample from the subject; determining if the subject has a metabolic defect related to disrupted bile acid anabolism, catabolism or homeostasis, or if the subject's gut microbiome has a defect related to disrupted bile acid anabolism, catabolism or homeostasis, or combinations thereof based on the measured concentration levels and calculated ratios of the one or more bile acid anabolism, catabolism or homeostasis biomarker metabolites in the sample as compared to a control sample; stratifying the subject into a subgroup of subjects, wherein an individual subgroup of subjects is defined by a unique and specific bile acid anabolism, catabolism or homeostasis profile based on the measured concentration levels and calculated ratios of the one or more biomarker metabolites or gut microbiome-related biomarker metabolites in the sample as compared to a control sample and the biomarker metabolite or gut microbiome-related biomarker metabolite defect determined for the subject. In one aspect, the method further comprises treating the depression, anxiety, mood disorders, or neuropsychiatric symptoms by administering to the subgroup of subjects an effective amount sufficient to attenuate, reduce, or eliminate the symptoms of depression, anxiety, mood disorders, or neuropsychiatric symptoms of a therapy to wherein the therapy is determined by the unique and specific metabolic profile of the subgroup of subjects. In another aspect, the one or more biomarker metabolites or gut microbiome-related biomarker metabolites comprises one or more of: Primary Bile Acids: cholic acid (CA); chenodeoxycholic acid (CDCA); chenodeoxycholic acid-24-acyl-β-d-glucuronide (CDCA-24G); 3β-cholic acid (β-CA); hyocholic acid (HCA); ω-monocarboxylic acid (ω-MCA); taurocholic acid (TCA); glycocholic acid (GCA); taurochenodeoxycholate (TCDCA); glycochenodeoxycholate (GCDCA); 5β-cholanic acid-3β,7β,12α-triol-5β-cholanic acid-3β, 7β, 12α-triol (β-UCA); taurohyocholate (THCA); glycohyocholate (GHCA); Secondary Bile Acids: glycohyodeoxycholate (GHDCA); deoxycholic acid (DCA); 23-nordeoxycholic acid (NorDCA); β-ursodeoxycholic acid (UDCA); lithocholic acid (LCA); 6,7-diketolithocholic acid (6,7-diketoLCA); 7-ketolithocholic acid (7-ketoLCA); lithocholic acid-3-sulfate (LCA-3S); hyodeoxycholic acid (HDCA); isolithocholic acid (isoLCA); β-hyodeoxycholic acid (isohyodeoxycholic acid; β-HDCA); allolithocholic acid (isoallolithocholic acid; alloLCA); dehydroLCA; 12-ketodeoxycholic acid (12-ketoLCA); 3β-ursodeoxycholic acid (isoursodeoxycholic acid; 3β-UDCA); glycodeoxycholic acid (GDCA); tauroursodeoxycholic acid (TUDCA); glycoursodeoxycholic acid (GUDCA); taurodeoxycholic acid (TDCA); glycolithocholic acid-3-sulfate (GLCA-3S); glycolithocholate (GLCA); taurohyodeoxycholic acid (THDCA); norcholic acid (NorCA); conjugated forms of typical bile acids; or combinations thereof. In another aspect, the method further comprises: administering to the subject a therapeutically effective amount of one or more primary or secondary bile acids and/or any pharmaceutically acceptable derivatives, esters, salts, solvates, hydrates, analogs, or prodrugs thereof; and/or administering to the subject a therapeutically effective amount of one or more therapeutic agents capable of modulating (increasing or decreasing) the concentration levels or ratios of one or more primary or secondary bile acids, activating the endogenous production of one or more primary or secondary bile acids, and/or decreasing the breakdown of one or more primary or secondary bile acids; and/or administering to the subject a therapeutically effective amount of one or more antidepressants selected from citalopram (Celexa®), escitalopram (Lexapro®), duloxetine (Cymbalta®), fluoxetine (Prozac®), paroxetine (Paxil®), sertraline (Zoloft®), trazodone (Desyrel®), lorazepam (Ativan®), oxazepam (Serax®), fluvoxamine (Luvox®), vilazodone (Viibryd®), vortioxetine (Trintellix®), aripiprazole (Abilify®), clozapine (Clozaril®), haloperidol (Haldol®), olanzapine (Zyprexa®), quetiapine (Seroquel®), risperidone (Risperdal®), ziprasidone (Geodon®), amitriptyline, amoxapine, desipramine (Norpramin®), doxepin, imipramine (Tofranil®), nortriptyline (Pamelor®), protriptyline, trimipramine, ketamine, or combinations thereof. In another aspect, the depression, anxiety, mood disorders, or neuropsychiatric symptoms are associated with neurological diseases or cognitive impairment, including dementia, vascular dementia, mixed dementia, early mild cognitive impairment (EMCI), late mild cognitive impairment (LMCI), Alzheimer's Disease, dementia with Lewy bodies, frontotemporal dementia, Creutzfeldt-Jakob disease, Parkinson's Disease, young-onset dementia, Korsakoff's syndrome, Huntington's disease, HIV-associated neurocognitive disorders, or other cognitive impairment disorders.

Another embodiment described herein is a method for detecting depression or anxiety in a subject, the method comprising: analyzing a sample from a subject; determining concentration levels or ratios of one or more biomarker metabolites or gut microbiome-related biomarker metabolites in the sample from the subject; and determining the subject as having depression or anxiety or an increased risk of depression or anxiety when the concentration levels or ratios of the one or more biomarker metabolites or gut microbiome-related biomarker metabolites in the sample from the subject are different from (greater than or less than) the concentration levels or ratios of the one or more biomarker metabolites or gut microbiome-related biomarker metabolites in a control sample. In another aspect, the method further comprises: initially treating the subject for depression or anxiety by administering an effective amount sufficient to attenuate, reduce, or eliminate the symptoms of depression or anxiety of one or more of antidepressants, cognitive behavior therapy, exercise, dietary supplements, prebiotics, probiotics, dietary changes, or an elimination diet; obtaining a second sample from the subject and determining the concentration levels or ratios of one or more biomarker metabolites or gut microbiome-related biomarker metabolites in the second sample from the subject; evaluating the concentration levels or ratios of the one or more biomarker metabolites or gut microbiome-related biomarker metabolites in comparison to control concentration levels or ratios of the one or more biomarker metabolites or gut microbiome-related biomarker metabolites; evaluating the efficacy of the depression or anxiety treatment; and continuing the one or more initial depression or anxiety treatments; administering one or more additional depression or anxiety treatments; or administering one or more second depression or anxiety treatments (switching the treatment regimen). In another aspect, the one or more biomarker metabolites or gut microbiome-related biomarker metabolites comprises one or more of: Primary Bile Acids: cholic acid (CA); chenodeoxycholic acid (CDCA); chenodeoxycholic acid-24-acyl-β-d-glucuronide (CDCA-24G); 3β-cholic acid (β-CA); hyocholic acid (HCA); ω-monocarboxylic acid (ω-MCA); taurocholic acid (TCA); glycocholic acid (GCA); taurochenodeoxycholate (TCDCA); glycochenodeoxycholate (GCDCA); 5β-cholanic acid-3β,7β,12α-triol-5β-cholanic acid-3β, 7β, 12α-triol (β-UCA): taurohyocholate (THCA); glycohyocholate (GHCA); Secondary Bile Acids: glycohyodeoxycholate (GHDCA); deoxycholic acid (DCA); 23-nordeoxycholic acid (NorDCA); β-ursodeoxycholic acid (UDCA); lithocholic acid (LCA); 6,7-diketolithocholic acid (6,7-diketoLCA); 7-ketolithocholic acid (7-ketoLCA); lithocholic acid-3-sulfate (LCA-3S); hyodeoxycholic acid (HDCA); isolithocholic acid (isoLCA); β-hyodeoxycholic acid (isohyodeoxycholic acid; β-HDCA); allolithocholic acid (isoallolithocholic acid; alloLCA); dehydroLCA; 12-ketodeoxycholic acid (12-ketoLCA); 3β-ursodeoxycholic acid (isoursodeoxycholic acid; 3β-UDCA); glycodeoxycholic acid (GDCA); tauroursodeoxycholic acid (TUDCA); glycoursodeoxycholic acid (GUDCA); taurodeoxycholic acid (TDCA); glycolithocholic acid-3-sulfate (GLCA-3S); glycolithocholate (GLCA); taurohyodeoxycholic acid (THDCA); norcholic acid (NorCA); conjugated forms of typical bile acids; or combinations thereof. In another aspect, the method further comprises: administering to the subject a therapeutically effective amount of one or more primary or secondary bile acids and/or any pharmaceutically acceptable derivatives, esters, salts, solvates, hydrates, analogs, or prodrugs thereof; and/or administering to the subject a therapeutically effective amount of one or more therapeutic agents capable of modulating (increasing or decreasing) the concentration levels or ratios of one or more primary or secondary bile acids, activating the endogenous production of one or more primary or secondary bile acids, and/or decreasing the breakdown of one or more primary or secondary bile acids. In another aspect, the biomarker metabolite or gut microbiome-related biomarker metabolite concentration level is greater than the control concentration level. In another aspect, the biomarker metabolite or gut microbiome-related biomarker metabolite concentration level is less than the control concentration level. In another aspect, two or more biomarker metabolite or gut microbiome-related biomarker metabolite concentration levels covary and are greater than the control concentration levels or covary and are less than the control concentration levels (positive correlation). In another aspect, two or more biomarker metabolite or gut microbiome-related biomarker metabolite concentration levels vary dissimilarly compared to the control concentration levels (negative correlation). In another aspect, the biomarker metabolite or gut microbiome-related biomarker metabolite comprises one or more primary bile acids selected from the group consisting of CDCA, CA, HCA, GHCA, and combinations thereof. In another aspect, when the biomarker metabolite or gut microbiome-related biomarker metabolite comprises CDCA, the concentration levels in the sample from the subject are less than the concentration levels in the control sample. In another aspect, the biomarker metabolite or gut microbiome-related biomarker metabolite comprises one or more secondary bile acids selected from the group consisting of UDCA, LCA, 7-ketoLCA, HDCA, isoLCA, β-HDCA, alloLCA, dehydroLCA, 12-ketoLCA, LCA-3S, GLCA-3S, HCA, NorCA, 6,7-diketoLCA, GDCA, β-UCA, and combinations thereof. In another aspect, when the biomarker metabolite or gut microbiome-related biomarker metabolite comprises one or more of LCA, isoLCA, alloLCA, 12-ketoLCA, LCA-3S, GLCA-3S, 7-ketoLCA, 6,7-diketoLCA, NorCA, GDCA, or β-HDCA, the concentration levels in the sample from the subject are greater than the concentration levels in the control sample. In another aspect, when the biomarker metabolite or gut microbiome-related biomarker metabolite comprises one or more of dehydroLCA, β-UCA, or HCA, the concentration levels in the sample from the subject are less than the concentration levels in the control sample. In another aspect, one or more ratios of the biomarker metabolites or gut microbiome-related biomarker metabolites are determined, the one or more ratios comprising a ratio of LCA/CDCA, 7-ketoLCA/CDCA, 12-ketoLCA/CDCA, GHCA/CDCA, HDCA/CDCA, ω-MCA/CDCA, alloLCA/CDCA, isoLCA/CDCA, LCA-3S/CDCA, GCA/TCA, TDCA/GDCA, THCA/GHCA, or combinations thereof. In another aspect, when the ratio of the biomarker metabolites or gut microbiome-related biomarker metabolites comprises one or more of LCA/CDCA, 7-ketoLCA/CDCA, 12-ketoLCA/CDCA, GHCA/CDCA, HDCA/CDCA, ω-MCA/CDCA, alloLCA/CDCA, isoLCA/CDCA, LCA-3S/CDCA, or GCA/TCA, the ratios in the sample from the subject are greater than the ratios in the control sample. In another aspect, when the ratio of the biomarker metabolites or gut microbiome-related biomarker metabolites comprises one or more of TDCA/GDCA or THCA/GHCA, the ratios in the sample from the subject are less than the ratios in the control sample. In another aspect, the sample from the subject is selected from one or more of whole blood, serum, plasma, urine, saliva, feces, or other body fluids. In another aspect, the control sample is from an untreated subject or a subject or a population of subjects not experiencing depression, anxiety, mood disorders, or neuropsychiatric symptoms or not at risk for depression, anxiety, mood disorders, or neuropsychiatric symptoms. In another aspect, the depression is Major Depression Disorder (MDD), core depression (CD+), anxious depression (ANX+), neurovegetative symptoms of melancholia (NVSM+), treatment resistant depression, subclinical characteristics associated with depression, or a neuropsychiatric symptom associated with a neurological disease or cognitive impairment. In another aspect, the depression, anxiety, mood disorders, or neuropsychiatric symptoms are associated with neurological diseases or cognitive impairment, including dementia, vascular dementia, mixed dementia, early mild cognitive impairment (EMCI), late mild cognitive impairment (LMCI), Alzheimer's Disease, dementia with Lewy bodies, frontotemporal dementia, Creutzfeldt-Jakob disease, Parkinson's Disease, young-onset dementia, Korsakoff's syndrome, Huntington's disease, HIV-associated neurocognitive disorders, or other cognitive impairment disorders. In another aspect, the antidepressant comprises citalopram (Celexa®), escitalopram (Lexapro®), duloxetine (Cymbalta®), fluoxetine (Prozac®), paroxetine (Paxil®), sertraline (Zoloft®), trazodone (Desyrel®), lorazepam (Ativan®), oxazepam (Serax®), fluvoxamine (Luvox®), vilazodone (Viibryd®), vortioxetine (Trintellix®), aripiprazole (Abilify®), clozapine (Clozaril®), haloperidol (Haldol®), olanzapine (Zyprexa®), quetiapine (Seroquel®), risperidone (Risperdal®), ziprasidone (Geodon®), amitriptyline, amoxapine, desipramine (Norpramin®), doxepin, imipramine (Tofranil®), nortriptyline (Pamelor®), protriptyline, trimipramine, ketamine, or combinations thereof. In another aspect, the antidepressant is one or more selective serotonin reuptake inhibitors (SSRI). In another aspect, the antidepressant comprises citalopram (Celexa®), escitalopram (Lexapro®), duloxetine (Cymbalta®), fluoxetine (Prozac®), paroxetine (Paxil®), sertraline (Zoloft®), paroxetine (Paxil®), fluvoxamine (Luvox®), vilazodone (Viibryd®), vortioxetine (Trintellix®), or other SSRI. In another aspect, the antidepressant comprises ketamine. In another aspect, the efficacy of the depression treatment is evaluated using the Hamilton Depression Rating Scale (HRSD₁₇), the Quick Inventory of Depressive Symptomatology (QIDS), subscales thereof, or specific questions thereof.

Another embodiment described herein is a method for treating depression, anxiety, mood disorders, or neuropsychiatric symptoms in a subject the method comprising: administering to the subject an effective amount sufficient to attenuate, reduce, or eliminate the symptoms of depression, anxiety, mood disorders, or neuropsychiatric symptoms of one or more of antidepressants, cognitive behavior therapy, exercise, dietary supplements, prebiotics, probiotics, dietary changes, or an elimination diet; analyzing sample from the subject; and measuring concentration levels or ratios in the subject's sample of one or more biomarker metabolites or gut microbiome-related biomarker metabolites comprising primary bile acids, secondary bile acids, or a combination thereof. In one aspect, the one or more biomarker metabolites or gut microbiome-related biomarker metabolites comprises one or more of: Primary Bile Acids: cholic acid (CA); chenodeoxycholic acid (CDCA); chenodeoxycholic acid-24-acyl-β-d-glucuronide (CDCA-24G); 3β-cholic acid (β-CA); hyocholic acid (HCA); ω-monocarboxylic acid (ω-MCA); taurocholic acid (TCA); glycocholic acid (GCA); taurochenodeoxycholate (TCDCA); glycochenodeoxycholate (GCDCA); 5β-cholanic acid-3β, 7β,12α-triol-5β-cholanic acid-3β, 7β, 12α-triol (β-UCA); taurohyocholate (THCA): glycohyocholate (GHCA); Secondary Bile Acids: glycohyodeoxycholate (GHDCA); deoxycholic acid (DCA); 23-nordeoxycholic acid (NorDCA); β-ursodeoxycholic acid (UDCA); lithocholic acid (LCA); 6,7-diketolithocholic acid (6,7-diketoLCA); 7-ketolithocholic acid (7-ketoLCA); lithocholic acid-3-sulfate (LCA-3S); hyodeoxycholic acid (HDCA); isolithocholic acid (isoLCA); β-hyodeoxycholic acid (isohyodeoxycholic acid; β-HDCA); allolithocholic acid (isoallolithocholic acid; alloLCA); dehydroLCA; 12-ketodeoxycholic acid (12-ketoLCA); 3β-ursodeoxycholic acid (isoursodeoxycholic acid; 3β-UDCA); glycodeoxycholic acid (GDCA); tauroursodeoxycholic acid (TUDCA); glycoursodeoxycholic acid (GUDCA); taurodeoxycholic acid (TDCA); glycolithocholic acid-3-sulfate (GLCA-3S); glycolithocholate (GLCA); taurohyodeoxycholic acid (THDCA); norcholic acid (NorCA); conjugated forms of typical bile acids; or combinations thereof. In another aspect, the method further comprises: administering to the subject a therapeutically effective amount of one or more primary or secondary bile acids and/or any pharmaceutically acceptable derivatives, esters, salts, solvates, hydrates, analogs, or prodrugs thereof; and/or administering to the subject a therapeutically effective amount of one or more therapeutic agents capable of modulating (increasing or decreasing) the concentration levels or ratios of one or more primary or secondary bile acids, activating the endogenous production of one or more primary or secondary bile acids, and/or decreasing the breakdown of one or more primary or secondary bile acids. In another aspect, the depression, anxiety, mood disorders, or neuropsychiatric symptoms are associated with neurological diseases or cognitive impairment, including dementia, vascular dementia, mixed dementia, early mild cognitive impairment (EMCI), late mild cognitive impairment (LMCI), Alzheimer's Disease, dementia with Lewy bodies, frontotemporal dementia, Creutzfeldt-Jakob disease, Parkinson's Disease, young-onset dementia, Korsakoff's syndrome, Huntington's disease, HIV-associated neurocognitive disorders, or other cognitive impairment disorders.

Another embodiment described herein is a method of treating depression, anxiety, mood disorders, or neuropsychiatric symptoms in a subject, the method comprising: administering to the subject an effective amount sufficient to attenuate, reduce, or eliminate the symptoms of depression, anxiety, mood disorders, or neuropsychiatric symptoms of one or more primary or secondary bile acids and/or any pharmaceutically acceptable derivatives, esters, salts, solvates, hydrates, analogs, or prodrugs thereof; and/or administering to the subject a therapeutically effective amount of one or more therapeutic agents capable of modulating (increasing or decreasing) the concentration levels or ratios of one or more primary or secondary bile acids, activating the endogenous production of one or more primary or secondary bile acids, and/or decreasing the breakdown of one or more primary or secondary bile acids. In one aspect, the depression, anxiety, mood disorders, or neuropsychiatric symptoms are associated with neurological diseases or cognitive impairment, including dementia, vascular dementia, mixed dementia, early mild cognitive impairment (EMCI), late mild cognitive impairment (LMCI), Alzheimer's Disease, dementia with Lewy bodies, frontotemporal dementia, Creutzfeldt-Jakob disease, Parkinson's Disease, young-onset dementia. Korsakoff's syndrome, Huntington's disease, HIV-associated neurocognitive disorders, or other cognitive impairment disorders.

Another embodiment described herein is a method for detecting and treating depression, anxiety, mood disorders, or neuropsychiatric symptoms in a subject, the method comprising: analyzing a sample from a subject; measuring concentration levels or ratios of one or more biomarker metabolites or gut microbiome-related biomarker metabolites comprising primary bile acids, secondary bile acids, or a combination thereof in the sample from the subject; determining the subject as having depression, anxiety, mood disorders, or neuropsychiatric symptoms or an increased risk of depression, anxiety, mood disorders, or neuropsychiatric symptoms when the concentration levels or ratios of the one or more biomarker metabolites or gut microbiome-related biomarker metabolites in the sample from the subject are different from (greater than or less than) the concentration levels or ratios of the one or more biomarker metabolites or gut microbiome-related biomarker metabolites in a control sample; and treating the subject by administering an effective amount sufficient to attenuate, reduce, or eliminate the symptoms of depression, anxiety, mood disorders, or neuropsychiatric symptoms of one or more primary or secondary bile acids and/or any pharmaceutically acceptable derivatives, esters, salts, solvates, hydrates, analogs, or prodrugs thereof, and/or administering an effective amount of one or more therapeutic agents capable of modulating (increasing or decreasing) the concentration levels or ratios of one or more primary or secondary bile acids, activating the endogenous production of one or more primary or secondary bile acids, and/or decreasing the breakdown of one or more primary or secondary bile acids. In one aspect, the one or more biomarker metabolites or gut microbiome-related biomarker metabolites comprises one or more of: Primary Bile Acids: cholic acid (CA); chenodeoxycholic acid (CDCA); chenodeoxycholic acid-24-acyl-β-d-glucuronide (CDCA-24G); 3β-cholic acid (β-CA); hyocholic acid (HCA); ω-monocarboxylic acid (ω-MCA); taurocholic acid (TCA); glycocholic acid (GCA); taurochenodeoxycholate (TCDCA); glycochenodeoxycholate (GCDCA); 5β-cholanic acid-3β, 7β,12α-triol-5β-cholanic acid-3β, 7β, 12α-triol (β-UCA); taurohyocholate (THCA); glycohyocholate (GHCA); Secondary Bile Acids: glycohyodeoxycholate (GHDCA); deoxycholic acid (DCA); 23-nordeoxycholic acid (NorDCA); β-ursodeoxycholic acid (UDCA); lithocholic acid (LCA); 6,7-diketolithocholic acid (6,7-diketoLCA); 7-ketolithocholic acid (7-ketoLCA); lithocholic acid-3-sulfate (LCA-3S); hyodeoxycholic acid (HDCA); isolithocholic acid (isoLCA); β-hyodeoxycholic acid (isohyodeoxycholic acid; β-HDCA); allolithocholic acid (isoallolithocholic acid; alloLCA); dehydroLCA; 12-ketodeoxycholic acid (12-ketoLCA); 3β-ursodeoxycholic acid (isoursodeoxycholic acid; 3β-UDCA); glycodeoxycholic acid (GDCA); tauroursodeoxycholic acid (TUDCA); glycoursodeoxycholic acid (GUDCA); taurodeoxycholic acid (TDCA); glycolithocholic acid-3-sulfate (GLCA-3S); glycolithocholate (GLCA); taurohyodeoxycholic acid (THDCA); norcholic acid (NorCA); conjugated forms of typical bile acids; or combinations thereof. In another aspect, the depression, anxiety, mood disorders, or neuropsychiatric symptoms are associated with neurological diseases or cognitive impairment, including dementia, vascular dementia, mixed dementia, early mild cognitive impairment (EMCI), late mild cognitive impairment (LMCI), Alzheimer's Disease, dementia with Lewy bodies, frontotemporal dementia, Creutzfeldt-Jakob disease, Parkinson's Disease, young-onset dementia, Korsakoff's syndrome, Huntington's disease, HIV-associated neurocognitive disorders, or other cognitive impairment disorders.

Another embodiment described herein is a method for isolating a biomarker metabolite useful for the analysis and identification of metabolic changes associated with depression, anxiety, mood disorders, or neuropsychiatric symptoms in a subject, the method comprising: analyzing a sample from a subject and a control subject or population of subjects with normal cognition; isolating one or more biomarker metabolites or gut microbiome-related biomarker metabolites in the sample from the subject and the control sample, wherein the one or more biomarker metabolites or gut microbiome-related biomarker metabolites are selected from the group consisting of a primary bile acid, a secondary bile acid, and combinations thereof; detecting and measuring concentration levels or ratios of the one or more biomarker metabolites or gut microbiome-related biomarker metabolites in the sample from the subject and the control sample using biochemical analysis; and determining the subject as having depression, anxiety, mood disorders, or neuropsychiatric symptoms or an increased risk of depression, anxiety, mood disorders, or neuropsychiatric symptoms when the concentration levels or ratios of the one or more biomarker metabolites or gut microbiome-related biomarker metabolites in the sample from the subject are different from (greater than or less than) the concentration levels or ratios of the one or more biomarker metabolites or gut microbiome-related biomarker metabolites in a control sample.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:
Clause 1. A method for the classification and treatment of depression, anxiety, mood disorders, or neuropsychiatric symptoms in a subject based on the subject's metabolic profile and genetic screening, the method comprising one or more of the following:
   identifying and stratifying subjects afflicted with a depression, anxiety, mood disorders, or neuropsychiatric symptoms to subgroups based on their metabolic profiles, biomarker metabolites and ratios of biomarker metabolites that define unique metabolic conditions related to aberrations in cholesterol metabolism or bile acid anabolism, catabolism homeostasis, or clearance and common identity among subgroups of subjects;
   evaluating the trajectory of disease within each stratified subgroup of subjects and their response to a therapeutic treatment;
   identifying defects in bile acid transport and/or biosynthesis of biomarker metabolites within a metabolic pathway or across metabolic pathways using ratios of biomarker metabolites to inform about changes in enzyme activities or transporters; and identifying genetic bases of bile acid metabolic profile characteristics or defects (SNPs/genetic variants in key enzymes and transporters) using metabolite genome-wide association study (mGWAS) analysis.
Clause 2. The method of clause 1, further comprising one or more of the following:
   using combined metabotype and genotype data to better stratify subjects with depression, anxiety, mood disorders, or neuropsychiatric symptoms and to inform about mechanisms and treatment selection;
   suggesting a therapeutic approach to correct metabolic defects in metabolic profile in stratified subgroups of subjects; and
   comparing and contrasting metabolic defects noted in inborn errors of metabolism that have depression, anxiety, mood disorders, or neuropsychiatric symptoms and using knowledge gained in treatment of inborn errors of metabolism to inform treatment for depression, anxiety, mood disorders, or neuropsychiatric symptoms.
Clause 3. The method of clause 1 or 2, further comprising performing imaging analysis on the subject and linking peripheral changes with brain changes.
Clause 4. The method of any one of clauses 1-3, further comprising administering to the subjects an effective amount of a therapy to prevent and/or treat the depression, anxiety, mood disorders, or neuropsychiatric symptoms affected by one or more metabolic defects.
Clause 5. The method of any one of clauses 1-4, wherein the depression, anxiety, mood disorders, or neuropsychiatric symptoms are associated with neurological diseases or cognitive impairment, including dementia, vascular dementia, mixed dementia, early mild cognitive impairment (EMCI), late mild cognitive impairment (LMCI), Alzheimer's Disease, dementia with Lewy bodies, frontotemporal dementia, Creutzfeldt-Jakob disease, Parkinson's Disease, young-onset dementia, Korsakoff's syndrome, Huntington's disease, HIV-associated neurocognitive disorders, or other cognitive impairment disorders.
Clause 6. A method for stratifying and treating a subject having a depression, anxiety, mood disorders, or neuropsychiatric symptoms, or at risk of developing a neurological disorder, based on the subject's metabolic profile, the method comprising:
   analyzing a sample from a subject to determine concentration levels or ratios of one or more biomarker metabolites or gut microbiome-related biomarker metabolites related to bile acid anabolism, catabolism or homeostasis in the sample from the subject;
   determining if the subject has a metabolic defect related to disrupted bile acid anabolism, catabolism or homeostasis, or if the subject's gut microbiome has a defect related to disrupted bile acid anabolism, catabolism or homeostasis, or combinations thereof based on the measured concentration levels and calculated ratios of the one or more bile acid anabolism, catabolism or homeostasis biomarker metabolites in the sample as compared to a control sample;
   stratifying the subject into a subgroup of subjects, wherein an individual subgroup of subjects is defined by a unique and specific bile acid anabolism, catabolism or homeostasis profile based on the measured concentration levels and calculated ratios of the one or more biomarker metabolites or gut microbiome-related biomarker metabolites in the sample as compared to a control sample and the biomarker metabolite or gut microbiome-related biomarker metabolite defect determined for the subject.
Clause 7. The method of clause 6, further comprising treating the depression, anxiety, mood disorders, or neuropsychiatric symptoms by administering to the subgroup of subjects an effective amount sufficient to attenuate, reduce, or eliminate the symptoms of depression, anxiety, mood disorders, or neuropsychiatric symptoms of a therapy to wherein the therapy is determined by the unique and specific metabolic profile of the subgroup of subjects.
Clause 8. The method of clause 6 or 7, wherein the one or more biomarker metabolites or gut microbiome-related biomarker metabolites comprises one or more of:
   Primary Bile Acids: cholic acid (CA); chenodeoxycholic acid (CDCA); chenodeoxycholic acid-24-acyl-β-d-glucuronide (CDCA-24G); 3β-cholic acid (β-CA); hyocholic acid (HCA); ω-monocarboxylic acid (ω-MCA); taurocholic acid (TCA); glycocholic acid (GCA); taurochenodeoxycholate (TCDCA); glycochenodeoxycholate (GCDCA); 5β-cholanic acid-3β,7β,12α-triol-5β-cholanic acid-3β, 7β, 12α-triol (β-UCA); taurohyocholate (THCA); glycohyocholate (GHCA);
   Secondary Bile Acids: glycohyodeoxycholate (GHDCA); deoxycholic acid (DCA); 23-nordeoxycholic acid (NorDCA); β-ursodeoxycholic acid (UDCA); lithocholic acid (LCA); 6,7-diketolithocholic acid (6,7-diketoLCA); 7-ketolithocholic acid (7-ketoLCA); lithocholic acid-3-sulfate (LCA-3S); hyodeoxycholic acid (HDCA); isolithocholic acid (isoLCA); β-hyodeoxycholic acid (isohyodeoxycholic acid; β-HDCA); allolithocholic acid (isoallolithocholic acid; alloLCA); dehydroLCA; 12-ketodeoxycholic acid (12-ketoLCA); 3β-ursodeoxycholic acid (isoursodeoxycholic acid; 3β-UDCA); glycodeoxycholic acid (GDCA); tauroursodeoxycholic acid (TUDCA); glycoursodeoxycholic acid (GUDCA); taurodeoxycholic acid (TDCA); glycolithocholic acid-3-sulfate (GLCA-3S); glycolithocholate (GLCA); taurohyodeoxycholic acid (THDCA); norcholic acid (NorCA);

conjugated forms of typical bile acids;

or combinations thereof.
Clause 9. The method of any one of clauses 6-8, further comprising:
   administering to the subject a therapeutically effective amount of one or more primary or secondary bile acids and/or any pharmaceutically acceptable derivatives, esters, salts, solvates, hydrates, analogs, or prodrugs thereof; and/or
   administering to the subject a therapeutically effective amount of one or more therapeutic agents capable of modulating (increasing or decreasing) the concentration levels or ratios of one or more primary or secondary bile acids, activating the endogenous production of one or more primary or secondary bile acids, and/or decreasing the breakdown of one or more primary or secondary bile acids; and/or
   administering to the subject a therapeutically effective amount of one or more antidepressants selected from citalopram (Celexa®), escitalopram (Lexapro®), duloxetine (Cymbalta®), fluoxetine (Prozac®), paroxetine (Paxil®), sertraline (Zoloft®), trazodone (Desyrel®), lorazepam (Ativan®), oxazepam (Serax®), fluvoxamine (Luvox®), vilazodone (Viibryd®), vortioxetine (Trintellix®), aripiprazole (Abilify®), clozapine (Clozaril®), haloperidol (Haldol®), olanzapine (Zyprexa®), quetiapine (Seroquel®), risperidone (Risperdal®), ziprasidone (Geodon®), amitriptyline, amoxapine, desipramine (Norpramin®), doxepin, imipramine (Tofranil®), nortriptyline (Pamelor®), protriptyline, trimipramine, ketamine, or combinations thereof.
Clause 10. The method of any one of clauses 6-9, wherein the depression, anxiety, mood disorders, or neuropsychiatric symptoms are associated with neurological diseases or cognitive impairment, including dementia, vascular dementia, mixed dementia, early mild cognitive impairment (EMCI), late mild cognitive impairment (LMCI), Alzheimer's Disease, dementia with Lewy bodies, frontotemporal dementia, Creutzfeldt-Jakob disease, Parkinson's Disease, young-onset dementia, Korsakoff's syndrome, Huntington's disease, HIV-associated neurocognitive disorders, or other cognitive impairment disorders.
Clause 11. A method for detecting depression or anxiety in a subject, the method comprising:
   analyzing a sample from a subject;
   determining concentration levels or ratios of one or more biomarker metabolites or gut microbiome-related biomarker metabolites in the sample from the subject; and
   determining the subject as having depression or anxiety or an increased risk of depression or anxiety when the concentration levels or ratios of the one or more biomarker metabolites or gut microbiome-related biomarker metabolites in the sample from the subject are different from (greater than or less than) the concentration levels or ratios of the one or more biomarker metabolites or gut microbiome-related biomarker metabolites in a control sample.
Clause 12. The method of clause 11, further comprising:
   initially treating the subject for depression or anxiety by administering an effective amount sufficient to attenuate, reduce, or eliminate the symptoms of depression or anxiety of one or more of antidepressants, cognitive behavior therapy, exercise, dietary supplements, prebiotics, probiotics, dietary changes, or an elimination diet;
   obtaining a second sample from the subject and determining the concentration levels or ratios of one or more biomarker metabolites or gut microbiome-related biomarker metabolites in the second sample from the subject;
   evaluating the concentration levels or ratios of the one or more biomarker metabolites or gut microbiome-related biomarker metabolites in comparison to control concentration levels or ratios of the one or more biomarker metabolites or gut microbiome-related biomarker metabolites;
   evaluating the efficacy of the depression or anxiety treatment; and
   continuing the one or more initial depression or anxiety treatments; administering one or more additional depression or anxiety treatments; or administering one or more second depression or anxiety treatments (switching the treatment regimen).
Clause 13. The method of any one of clauses 11 or 12, wherein the one or more biomarker metabolites or gut microbiome-related biomarker metabolites comprises one or more of: Primary Bile Acids: cholic acid (CA); chenodeoxycholic acid (CDCA); chenodeoxycholic acid-24-acyl-β-d-glucuronide (CDCA-24G); 3β-cholic acid (β-CA); hyocholic acid (HCA); ω-monocarboxylic acid (ω-MCA); taurocholic acid (TCA); glycocholic acid (GCA); taurochenodeoxycholate (TCDCA); glycochenodeoxycholate (GCDCA); 5β-cholanic acid-3β,7β,12α-triol-5β-cholanic acid-3β, 7β, 12α-triol (β-UCA); taurohyocholate (THCA); glycohyocholate (GHCA);
   Secondary Bile Acids: glycohyodeoxycholate (GHDCA); deoxycholic acid (DCA); 23-nordeoxycholic acid (NorDCA); β-ursodeoxycholic acid (UDCA); lithocholic acid (LCA); 6,7-diketolithocholic acid (6,7-diketoLCA); 7-ketolithocholic acid (7-ketoLCA); lithocholic acid-3-sulfate (LCA-3S); hyodeoxycholic acid (HDCA); isolithocholic acid (isoLCA); β-hyodeoxycholic acid (isohyodeoxycholic acid; β-HDCA); allolithocholic acid (isoallolithocholic acid; alloLCA); dehydroLCA; 12-ketodeoxycholic acid (12-ketoLCA); 3β-ursodeoxycholic acid (isoursodeoxycholic acid; 3β-UDCA); glycodeoxycholic acid (GDCA); tauroursodeoxycholic acid (TUDCA); glycoursodeoxycholic acid (GUDCA); taurodeoxycholic acid (TDCA); glycolithocholic acid-3-sulfate (GLCA-3S); glycolithocholate (GLCA); taurohyodeoxycholic acid (THDCA); norcholic acid (NorCA);
   conjugated forms of typical bile acids;
   or combinations thereof.
Clause 14. The method of any one of clauses 11-13, further comprising:
   administering to the subject a therapeutically effective amount of one or more primary or secondary bile acids and/or any pharmaceutically acceptable derivatives, esters, salts, solvates, hydrates, analogs, or prodrugs thereof; and/or
   administering to the subject a therapeutically effective amount of one or more therapeutic agents capable of modulating (increasing or decreasing) the concentration levels or ratios of one or more primary or secondary bile acids, activating the endogenous production of one or more primary or secondary bile acids, and/or decreasing the breakdown of one or more primary or secondary bile acids.
Clause 15. The method of any one of clauses 11-14, wherein the biomarker metabolite or gut microbiome-related biomarker metabolite concentration level is greater than the control concentration level.
Clause 16. The method of any one of clauses 11-15, wherein the biomarker metabolite or gut microbiome-related biomarker metabolite concentration level is less than the control concentration level.
Clause 17. The method of any one of clauses 11-16, wherein two or more biomarker metabolite or gut microbiome-related biomarker metabolite concentration levels covary and are greater than the control concentration levels or covary and are less than the control concentration levels (positive correlation).
Clause 18. The method of any one of clauses 11-17, wherein two or more biomarker metabolite or gut microbiome-related biomarker metabolite concentration levels vary dissimilarly compared to the control concentration levels (negative correlation).
Clause 19. The method of any one of clauses 11-18, wherein the biomarker metabolite or gut microbiome-related biomarker metabolite comprises one or more primary bile acids selected from the group consisting of CDCA, CA, HCA, GHCA, and combinations thereof.
Clause 20. The method of any one of clauses 11-19, wherein when the biomarker metabolite or gut microbiome-related biomarker metabolite comprises CDCA, the concentration levels in the sample from the subject are less than the concentration levels in the control sample.
Clause 21. The method of any one of clauses 11-20, wherein the biomarker metabolite or gut microbiome-related biomarker metabolite comprises one or more secondary bile acids selected from the group consisting of UDCA, LCA, 7-ketoLCA, HDCA, isoLCA, β-HDCA, alloLCA, dehydroLCA, 12-ketoLCA, LCA-3S, GLCA-3S, HCA, NorCA, 6,7-diketoLCA, GDCA, β-UCA, and combinations thereof.
Clause 22. The method of any one of clauses 11-21, wherein when the biomarker metabolite or gut microbiome-related biomarker metabolite comprises one or more of LCA, isoLCA, alloLCA, 12-ketoLCA, LCA-3S, GLCA-3S, 7-ketoLCA, 6,7-diketoLCA, NorCA, GDCA, or β-HDCA, the concentration levels in the sample from the subject are greater than the concentration levels in the control sample.
Clause 23. The method of any one of clauses 11-22, wherein when the biomarker metabolite or gut microbiome-related biomarker metabolite comprises one or more of dehydroLCA, β-UCA, or HCA, the concentration levels in the sample from the subject are less than the concentration levels in the control sample.
Clause 24. The method of any one of clauses 11-23, wherein one or more ratios of the biomarker metabolites or gut microbiome-related biomarker metabolites are determined, the one or more ratios comprising a ratio of LCA/CDCA, 7-ketoLCA/CDCA, 12-ketoLCA/CDCA, GHCA/CDCA, HDCA/CDCA, W-MCA/CDCA, alloLCA/CDCA, isoLCA/CDCA, LCA-3S/CDCA, GCA/TCA, TDCA/GDCA, THCA/GHCA, or combinations thereof.
Clause 25. The method of any one of clauses 11-24, wherein when the ratio of the biomarker metabolites or gut microbiome-related biomarker metabolites comprises one or more of LCA/CDCA, 7-ketoLCA/CDCA, 12-ketoLCA/CDCA, GHCA/CDCA, HDCA/CDCA, ω-MCA/CDCA, alloLCA/CDCA, isoLCA/CDCA, LCA-3S/CDCA, or GCA/TCA, the ratios in the sample from the subject are greater than the ratios in the control sample.
Clause 26. The method of any one of clauses 11-25, wherein when the ratio of the biomarker metabolites or gut microbiome-related biomarker metabolites comprises one or more of TDCA/GDCA or THCA/GHCA, the ratios in the sample from the subject are less than the ratios in the control sample.
Clause 27. The method of any one of clauses 11-26, wherein the sample from the subject is selected from one or more of whole blood, serum, plasma, urine, saliva, feces, or other body fluids.
Clause 28. The method of any one of clauses 11-27, wherein the control sample is from an untreated subject or a subject or a population of subjects not experiencing depression, anxiety, mood disorders, or neuropsychiatric symptoms or not at risk for depression, anxiety, mood disorders, or neuropsychiatric symptoms.
Clause 29. The method of any one of clauses 11-28, wherein the depression is Major Depression Disorder (MDD), core depression (CD+), anxious depression (ANX+), neurovegetative symptoms of melancholia (NVSM+), treatment resistant depression, subclinical characteristics associated with depression, or a neuropsychiatric symptom associated with a neurological disease or cognitive impairment.
Clause 30. The method of any one of clauses 11-29, wherein the depression, anxiety, mood disorders, or neuropsychiatric symptoms are associated with neurological diseases or cognitive impairment, including dementia, vascular dementia, mixed dementia, early mild cognitive impairment (EMCI), late mild cognitive impairment (LMCI), Alzheimer's Disease, dementia with Lewy bodies, frontotemporal dementia, Creutzfeldt-Jakob disease, Parkinson's Disease, young-onset dementia, Korsakoff's syndrome, Huntington's disease, HIV-associated neurocognitive disorders, or other cognitive impairment disorders.
Clause 31. The method of any one of clauses 11-30, wherein the antidepressant comprises citalopram (Celexa®), escitalopram (Lexapro®), duloxetine (Cymbalta®), fluoxetine (Prozac®), paroxetine (Paxil®), sertraline (Zoloft®), trazodone (Desyrel®), lorazepam (Ativan®), oxazepam (Serax®), fluvoxamine (Luvox®), vilazodone (Viibryd®), vortioxetine (Trintellix®), aripiprazole (Abilify®), clozapine (Clozaril®), haloperidol (Haldol®), olanzapine (Zyprexa®), quetiapine (Seroquel®), risperidone (Risperdal®), ziprasidone (Geodon®), amitriptyline, amoxapine, desipramine (Norpramin®), doxepin, imipramine (Tofranil®), nortriptyline (Pamelor®), protriptyline, trimipramine, ketamine, or combinations thereof.
Clause 32. The method of any one of clauses 11-31, wherein the antidepressant is one or more selective serotonin reuptake inhibitors (SSRI).
Clause 33. The method of any one of clauses 11-32, wherein the antidepressant comprises citalopram (Celexa®), escitalopram (Lexapro®), duloxetine (Cymbalta®), fluoxetine (Prozac®), paroxetine (Paxil®), sertraline (Zoloft®), paroxetine (Paxil®), fluvoxamine (Luvox®), vilazodone (Viibryd®), vortioxetine (Trintellix®), or other SSRI.
Clause 34. The method of any one of clauses 11-33, wherein the antidepressant comprises ketamine.
Clause 35. The method of any one of clauses 11-34, wherein the efficacy of the depression treatment is evaluated using the Hamilton Depression Rating Scale (HRSD₁₇), the Quick Inventory of Depressive Symptomatology (QIDS), subscales thereof, or specific questions thereof.
Clause 36. A method for treating depression, anxiety, mood disorders, or neuropsychiatric symptoms in a subject the method comprising:
   administering to the subject an effective amount sufficient to attenuate, reduce, or eliminate the symptoms of depression, anxiety, mood disorders, or neuropsychiatric symptoms of one or more of antidepressants, cognitive behavior therapy, exercise, dietary supplements, prebiotics, probiotics, dietary changes, or an elimination diet;
   analyzing sample from the subject; and
   measuring concentration levels or ratios in the subject's sample of one or more biomarker metabolites or gut microbiome-related biomarker metabolites comprising primary bile acids, secondary bile acids, or a combination thereof.
Clause 37. The method of clause 36, wherein the one or more biomarker metabolites or gut microbiome-related biomarker metabolites comprises one or more of:
   Primary Bile Acids: cholic acid (CA); chenodeoxycholic acid (CDCA); chenodeoxycholic acid-24-acyl-β-d-glucuronide (CDCA-24G); 3β-cholic acid (β-CA); hyocholic acid (HCA); ω-monocarboxylic acid (ω-MCA); taurocholic acid (TCA); glycocholic acid (GCA); taurochenodeoxycholate (TCDCA); glycochenodeoxycholate (GCDCA); 5β-cholanic acid-3β,7β,12α-triol-5β-cholanic acid-3β, 7β, 12α-triol (β-UCA); taurohyocholate (THCA); glycohyocholate (GHCA);
   Secondary Bile Acids: glycohyodeoxycholate (GHDCA); deoxycholic acid (DCA); 23-nordeoxycholic acid (NorDCA); β-ursodeoxycholic acid (UDCA); lithocholic acid (LCA); 6,7-diketolithocholic acid (6,7-diketoLCA); 7-ketolithocholic acid (7-ketoLCA); lithocholic acid-3-sulfate (LCA-3S); hyodeoxycholic acid (HDCA); isolithocholic acid (isoLCA); β-hyodeoxycholic acid (isohyodeoxycholic acid; β-HDCA); allolithocholic acid (isoallolithocholic acid; alloLCA); dehydroLCA; 12-ketodeoxycholic acid (12-ketoLCA); 3β-ursodeoxycholic acid (isoursodeoxycholic acid: 3β-UDCA); glycodeoxycholic acid (GDCA); tauroursodeoxycholic acid (TUDCA); glycoursodeoxycholic acid (GUDCA); taurodeoxycholic acid (TDCA); glycolithocholic acid-3-sulfate (GLCA-3S); glycolithocholate (GLCA); taurohyodeoxycholic acid (THDCA); norcholic acid (NorCA);
   conjugated forms of typical bile acids;
   or combinations thereof.
Clause 38. The method of any one of clauses 36 or 37, further comprising:
   administering to the subject a therapeutically effective amount of one or more primary or secondary bile acids and/or any pharmaceutically acceptable derivatives, esters, salts, solvates, hydrates, analogs, or prodrugs thereof; and/or
   administering to the subject a therapeutically effective amount of one or more therapeutic agents capable of modulating (increasing or decreasing) the concentration levels or ratios of one or more primary or secondary bile acids, activating the endogenous production of one or more primary or secondary bile acids, and/or decreasing the breakdown of one or more primary or secondary bile acids.
Clause 39. The method of any one of clauses 36-38, wherein the depression, anxiety, mood disorders, or neuropsychiatric symptoms are associated with neurological diseases or cognitive impairment, including dementia, vascular dementia, mixed dementia, early mild cognitive impairment (EMCI), late mild cognitive impairment (LMCI), Alzheimer's Disease, dementia with Lewy bodies, frontotemporal dementia, Creutzfeldt-Jakob disease, Parkinson's Disease, young-onset dementia, Korsakoff's syndrome, Huntington's disease, HIV-associated neurocognitive disorders, or other cognitive impairment disorders.
Clause 40. A method of treating depression, anxiety, mood disorders, or neuropsychiatric symptoms in a subject, the method comprising:
   administering to the subject an effective amount sufficient to attenuate, reduce, or eliminate the symptoms of depression, anxiety, mood disorders, or neuropsychiatric symptoms of one or more primary or secondary bile acids and/or any pharmaceutically acceptable derivatives, esters, salts, solvates, hydrates, analogs, or prodrugs thereof; and/or
   administering to the subject a therapeutically effective amount of one or more therapeutic agents capable of modulating (increasing or decreasing) the concentration levels or ratios of one or more primary or secondary bile acids, activating the endogenous production of one or more primary or secondary bile acids, and/or decreasing the breakdown of one or more primary or secondary bile acids.
Clause 41. The method of clause 40, wherein the depression, anxiety, mood disorders, or neuropsychiatric symptoms are associated with neurological diseases or cognitive impairment, including dementia, vascular dementia, mixed dementia, early mild cognitive impairment (EMCI), late mild cognitive impairment (LMCI), Alzheimer's Disease, dementia with Lewy bodies, frontotemporal dementia, Creutzfeldt-Jakob disease, Parkinson's Disease, young-onset dementia, Korsakoff's syndrome, Huntington's disease, HIV-associated neurocognitive disorders, or other cognitive impairment disorders.
Clause 42. A method for detecting and treating depression, anxiety, mood disorders, or neuropsychiatric symptoms in a subject, the method comprising:
   analyzing a sample from a subject;
   measuring concentration levels or ratios of one or more biomarker metabolites or gut microbiome-related biomarker metabolites comprising primary bile acids, secondary bile acids, or a combination thereof in the sample from the subject;
   determining the subject as having depression, anxiety, mood disorders, or neuropsychiatric symptoms or an increased risk of depression, anxiety, mood disorders, or neuropsychiatric symptoms when the concentration levels or ratios of the one or more biomarker metabolites or gut microbiome-related biomarker metabolites in the sample from the subject are different from (greater than or less than) the concentration levels or ratios of the one or more biomarker metabolites or gut microbiome-related biomarker metabolites in a control sample; and
   treating the subject by administering an effective amount sufficient to attenuate, reduce, or eliminate the symptoms of depression, anxiety, mood disorders, or neuropsychiatric symptoms of one or more primary or secondary bile acids and/or any pharmaceutically acceptable derivatives, esters, salts, solvates, hydrates, analogs, or prodrugs thereof, and/or administering an effective amount of one or more therapeutic agents capable of modulating (increasing or decreasing) the concentration levels or ratios of one or more primary or secondary bile acids, activating the endogenous production of one or more primary or secondary bile acids, and/or decreasing the breakdown of one or more primary or secondary bile acids.
Clause 43. The method of clause 42, wherein the one or more biomarker metabolites or gut microbiome-related biomarker metabolites comprises one or more of:
   Primary Bile Acids: cholic acid (CA); chenodeoxycholic acid (CDCA); chenodeoxycholic acid-24-acyl-β-d-glucuronide (CDCA-24G); 3β-cholic acid (β-CA); hyocholic acid (HCA); ω-monocarboxylic acid (ω-MCA); taurocholic acid (TCA); glycocholic acid (GCA); taurochenodeoxycholate (TCDCA); glycochenodeoxycholate (GCDCA); 5β-cholanic acid-3β, 7β,12α-triol-5β-cholanic acid-3β, 7β, 12α-triol (β-UCA); taurohyocholate (THCA); glycohyocholate (GHCA);
   Secondary Bile Acids: glycohyodeoxycholate (GHDCA); deoxycholic acid (DCA); 23-nordeoxycholic acid (NorDCA); β-ursodeoxycholic acid (UDCA); lithocholic acid (LCA); 6,7-diketolithocholic acid (6,7-diketoLCA); 7-ketolithocholic acid (7-ketoLCA); lithocholic acid-3-sulfate (LCA-3S); hyodeoxycholic acid (HDCA); isolithocholic acid (isoLCA); β-hyodeoxycholic acid (isohyodeoxycholic acid; β-HDCA); allolithocholic acid (isoallolithocholic acid; alloLCA); dehydroLCA; 12-ketodeoxycholic acid (12-ketoLCA); 3β-ursodeoxycholic acid (isoursodeoxycholic acid; 3β-UDCA); glycodeoxycholic acid (GDCA); tauroursodeoxycholic acid (TUDCA); glycoursodeoxycholic acid (GUDCA); taurodeoxycholic acid (TDCA); glycolithocholic acid-3-sulfate (GLCA-3S); glycolithocholate (GLCA); taurohyodeoxycholic acid (THDCA); norcholic acid (NorCA);
   conjugated forms of typical bile acids;
   or combinations thereof.
Clause 44. The method of any one of clauses 42 or 43, wherein the depression, anxiety, mood disorders, or neuropsychiatric symptoms are associated with neurological diseases or cognitive impairment, including dementia, vascular dementia, mixed dementia, early mild cognitive impairment (EMCI), late mild cognitive impairment (LMCI), Alzheimer's Disease, dementia with Lewy bodies, frontotemporal dementia, Creutzfeldt-Jakob disease, Parkinson's Disease, young-onset dementia, Korsakoff's syndrome, Huntington's disease, HIV-associated neurocognitive disorders, or other cognitive impairment disorders.
Clause 45. A method for isolating a biomarker metabolite useful for the analysis and identification of metabolic changes associated with depression, anxiety, mood disorders, or neuropsychiatric symptoms in a subject, the method comprising:
   analyzing a sample from a subject and a control subject or population of subjects with normal cognition;
   isolating one or more biomarker metabolites or gut microbiome-related biomarker metabolites in the sample from the subject and the control sample, wherein the one or more biomarker metabolites or gut microbiome-related biomarker metabolites are selected from the group consisting of a primary bile acid, a secondary bile acid, and combinations thereof;
   detecting and measuring concentration levels or ratios of the one or more biomarker metabolites or gut microbiome-related biomarker metabolites in the sample from the subject and the control sample using biochemical analysis; and
   determining the subject as having depression, anxiety, mood disorders, or neuropsychiatric symptoms or an increased risk of depression, anxiety, mood disorders, or neuropsychiatric symptoms when the concentration levels or ratios of the one or more biomarker metabolites or gut microbiome-related biomarker metabolites in the sample from the subject are different from (greater than or less than) the concentration levels or ratios of the one or more biomarker metabolites or gut microbiome-related biomarker metabolites in a control sample.

### EXAMPLES

### Example 1

### Study Design and Participants

This study examined serum samples from the Predictors of Remission in Depression to Individual and Combined Treatments (PREDICT) study. The design and clinical outcomes of PREDICT have been described previously. PREDICT aimed to identify predictors and moderators of response to 12 weeks of randomly assigned treatment with duloxetine (30-60 mg/day), escitalopram (10-20 mg/day) or cognitive behavior therapy (16 one-hour individual sessions). Eligible participants were adults aged 18-65 with nonpsychotic MDD who had never previously been treated for depression. Severity of depression at the randomization visit was assessed with the 17-item Hamilton Depression Rating Scale (HRSD₁₇). Eligibility required an HRSD₁₇ score ≥18 at the screening visit and >15 at the randomization visit, indicative of moderate-to-severe depression. Patients were excluded if they had a history of bipolar disorder, neurocognitive disorder, or anorexia nervosa, or had an active significant suicide risk, current illicit drug use (assessed by history and with urine drug screen) or a history of substance abuse in the three months prior to randomization, pregnancy, lactation, or any uncontrolled general medical condition.

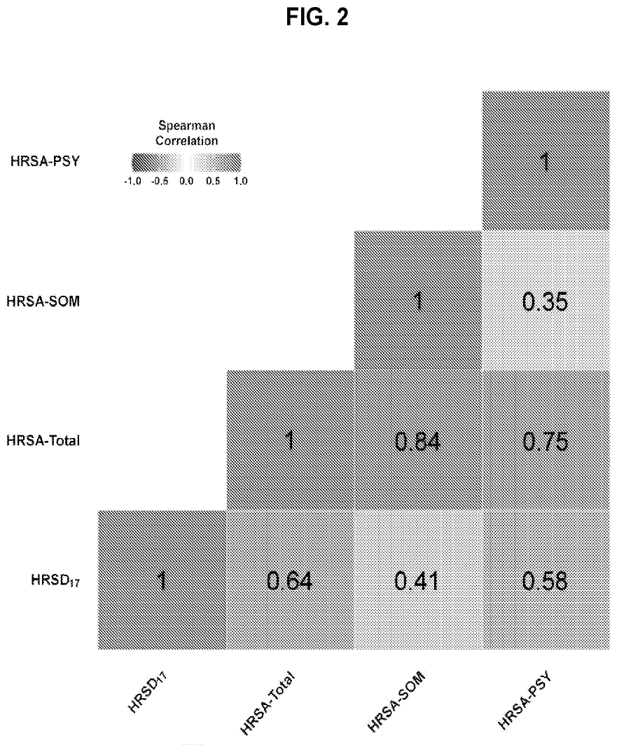

Participant Characteristics (Demographic and Clinical) Table 1 summarizes the demographic and clinical features of the 208 participants in the PREDICT Study. Of these, 38.94% of participants were male, and mean (standard error of mean) age, HRSD₁₇, and HRSA-Total were 38.99 (+0.81), 19.89 (+0.26), and 16.40 (+0.37), respectively. Baseline total HRSD₁₇ scores were highly correlated with HRSA-Total scores (Spearman rank correlation rho=0.64) and HRSA-PSY (rho=0.58), but less strongly correlated with HRSA-SOM (rho=0.41). The correlation between HRSA-PSY and HRSA-SOM was only rho=0.35 (FIG. 2).

TABLE 1
Participant Demographic and Clinical Characteristics

| | | | Depression | | | | | | | | Treatment Outcome | | | |
|---|---|---|---|---|---|---|---|---|---|---|---|---|---|---|
| | | | Non- | | | | Anxiety | | | | | | Treatment | |
| | Population | | Severe | | Severe | | Low | | High | | Remission | | Failure | |
| Characteristic | (N = 208) | | (N = 102) | | (N = 106) | | (N = 91) | | (N = 117) | | (N = 73) | | (N = 25) | |
| Age^{a} | 38.99 | (0.81) | 36.93 | (1.14) | 40.97 | (1.13) | 38.77 | (1.27) | 46 | (50.55) | 37.40 | (1.24) | 37.68 | (2.61) |
| Sex: | | | | | | | | | | | | | | |
| Male^{b} | 81 | (38.94%) | 46 | (45.10%) | 35 | (33.02%) | 46 | (50.55%) | 35 | (29.91%) | 32 | (43.84%) | 10 | (40%) |
| Body | | | | | | | | | | | | | | |
| Mass | 28.78 | (0.42) | 28.59 | (0.65) | 28.97 | (0.55) | 28.34 | (0.69) | 29.13 | (0.53) | 29.18 | (0.75) | 27.62 | (1.14) |
| Index^{a} | | | | | | | | | | | | | | |
| HRSD₁₇^{a} | 19.89 | (0.26) | 16.82 | (0.14) | 22.84 | (0.28) | 17.69 | (0.29) | 21.60 | (0.34) | 18.56 | (0.42) | 19.20 | (0.69) |
| HRSA- | 16.40 | (0.37) | 13.46 | (0.38) | 19.24 | (0.49) | 11.77 | (0.21) | 20.01 | (0.39) | 14.78 | (0.55) | 15.80 | (1.09) |
| Total^{a} | | | | | | | | | | | | | | |
| HRSA- | 4.04 | (0.23) | 2.77 | (0.26) | 5.25 | (0.34) | 1.64 | (0.16) | 5.91 | (0.29) | 3.21 | (0.34) | 3.88 | (0.78) |
| SOM^{a} | | | | | | | | | | | | | | |
| HRSA- | 10.84 | (0.19) | 9.50 | (0.21) | 12.12 | (0.25) | 8.98 | (0.19) | 12.28 | (0.22) | 10.04 | (0.26) | 10.48 | (0.52) |
| PSY^{a} | | | | | | | | | | | | | | |

^{a}Mean and standard error of the mean for each group
^{b}Number and percent of males for each group
Abbreviations: HRSA-SOM: Somatic anxiety subscore of the Hamilton Anxiety Rating Scale; HRSA-PSY: Psychic anxiety subscore of the Hamilton Anxiety Rating Scale; HRSA-Total: 14-item Hamilton Anxiety Rating Scale; HRSD₁₇: 17-item Hamilton Depression Rating Scale.

### Metabolomic Profiling and Ratios and Summations

At the randomization visit, antecubital phlebotomy was performed without regard for time of day or fasting status to obtain the serum samples used in the current analysis. Blood samples were allowed to clot for 20 minutes, then centrifuged at 4° C. for 10 minutes. The serum was pipetted into Eppendorf tubes and immediately frozen at −80° C. until ready for metabolomic analysis. Using targeted metabolomics protocols and profiling protocols established in previous studies, BAs were quantified by ultra-performance liquid chromatography triple quadrupole mass spectrometry (Waters XEVO TQ-S, Milford, USA). BAs primary and secondary conjugated and unconjugated measured are shown in Table 2.

Individual BAs were examined as well as a number of BA summations and ratios that have been previously implicated in several pathophysiological conditions. See Table 3 for these ratios and their associated diseases or metabolic conditions.

### Depression and Anxiety Symptoms

Depression severity was assessed using the clinician-administered HRSD₁₇ and the patient-completed 16-item Quick Inventory of Depressive Symptomatology, Self-Report. Participants with HRSD₁₇<20 were labeled as non-severely depressed and those with HRSD₁₇≥20 as severely depressed. Anxiety symptom severity was assessed using the clinician-rated 14-item Hamilton Anxiety Rating Scale (HRSA-Total), which consists of two subscales: “psychic anxiety” (items 1-6 and 14) (HRSA-PSY), and “somatic anxiety” (items 7-13) (HRSA-SOM). Psychic anxiety (HRSA-PSY) consists of the symptoms of anxious mood, tension, fears, depressed mood, insomnia, impaired concentration, and restlessness. Somatic anxiety (HRSA-SOM) consists of physical symptoms associated with the muscular, sensory, cardiovascular, respiratory, gastrointestinal, genitourinary, and autonomic systems. Participants were divided into those with high (HRSA-Total ≥15) and low (HRSA-Total <15) levels of anxiety. The HRSD₁₇, 16-item Quick Inventory of Depressive Symptomatology, Self-Report, and HRSA-Total ratings were re-administered after the completion of treatment at week 12. Consistent with other studies evaluating the biological effects of treatments, the participants who achieved remission (remitters) (defined as completing 12 weeks of treatment and reaching HRSD₁₇≤7) were compared to those who completed 12 weeks of treatment but whose week 12 HRSD₁₇ score was <30% lower than their baseline score (treatment failure).

### Statistical Analyses

Differences in demographic variables and depression scores across the response groups were evaluated using ANOVA and the Pearson Chi-squared test (for categorical variables). All analyses were performed in a metabolite-wise manner in two ways: (1) Difference in metabolite concentrations in severe vs. non-severe depression, high vs. low anxiety levels, and remission vs. treatment failure were analyzed using the nonparametric, two-sample Wilcoxon signed-rank test; and (2) Partial correlations between metabolite levels and the continuous variables HRSD₁₇, HRSA-Total, HRSA-SOM, and HRSA-PSY were conducted using partial Spearman rank correlation and adjusted for age, sex, and body mass index. A p-value <0.05 was considered significant. Given the exploratory nature of this initial investigation, no correction for multiple comparisons was made.

Separate partial least squares regression and partial least squares discriminant analysis were conducted to examine the contribution of baseline BA levels to baseline HRSD₁₇, HRSA-Total, and treatment outcome. In all models, age, sex, and body mass index were accounted, using 5-fold cross-validation with 100 repeats. In partial least squares regression models, baseline BA profiles of all participants were considered as predictor variables, and the HRSD₁₇ and HRSA-Total as continuous dependent variables. Using a partial least squares discriminant analysis model, it was examined whether the baseline BA profiles could discriminate participants at the two extremes of the treatment response spectrum, the remitters and those with treatment failure. Significant predictors were identified based on their variable importance on projection scores. Variables with a variable importance on projection score value >1 were considered important for the models.

### Partial Least-Squares Regression and Discriminant Analysis

Separate partial least squares regression (PLS-regression) and discriminant analysis (PLSDA) were performed to examine the contribution of baseline bile acid (BA) levels to baseline 17-item Hamilton Depression Rating Scale (HRSD₁₇) scores, clinician-rated 14-item Hamilton Anxiety Rating Scale (HRSA-Total) scores, and treatment outcome. In all models, age, sex, and body mass index were considered using 5-fold cross-validation with 100 repeats. In PLS-regression models, baseline BA profiles of all participants were considered as predictor variables, and HRSD₁₇ and HRSA-Total as continuous dependent variables. Using a PLSDA model, it was determined whether the baseline BA profiles could discriminate participants at the two extremes of the treatment response spectrum, the remitters and those with treatment failure. Significant predictors were identified based on their variable importance on projection (VIP) scores. Variables with a VIP value >1 were considered important for the models.

### BA Profiles Related to Depressive Symptom Severity

The concentrations of the conjugated and unconjugated versions of the primary and secondary BAs are reported in Table 2.

TABLE 2
Serum Levels of Bile Acids Primary, Secondary and their Conjugated Forms in Patients
Enrolled in the PReDICT Study. All Values are at Baseline Prior to Treatment.

| Metabolite | Abbreviate | HMDB ID | Type | Conjugation? |
|---|---|---|---|---|
| Cholic Acid | CA | HMDB00619 | Primary | Unconjugated |
| Chenodeoxycholic Acid | CDCA | HMDB00518 | Primary | Unconjugated |
| Chenodeoxycholic Acid | CDCA_24G | NA | Primary | Unconjugated |
| 24 Acyl β D | | | | |
| glucuronide | | | | |
| 3β Cholic Acid | βCA | HMDB00419 | Primary | Unconjugated |
| Hyocholic Acid | HCA | HMDB00760 | Primary | Unconjugated |
| ω Muricholic Acid | ωMCA | HMDB00364 | Primary | Unconjugated |
| Nor Cholic acid | NorCA | NA | Primary | Unconjugated |
| Taurocholic Acid | TCA | HMDB00036 | Primary | Conjugated |
| Glycocholic Acid | GCA | HMDB00138 | Primary | Conjugated |
| Taurochenodeoxycholate | TCDCA | HMDB00951 | Primary | Conjugated |
| Glycochenodeoxycholate | GCDCA | HMDB00637 | Primary | Conjugated |
| 5β cholanic acid | | | | |
| 3β,7β,12α | | | | |
| triol 5β | | | | |
| Cholanic Acid | βUCA | M07X096_N | Primary | Unconjugated |
| 3β,7β,12α | | | | |
| triol | | | | |
| Taurohyocholate | THCA | HMDB11637 | Primary | Conjugated |
| Glycohyocholate | GHCA | M07X072_N | Primary | Conjugated |
| Glycohyodeoxycholate | GHDCA | NA | Secondary | Conjugated |
| Deoxycholic Acid | DCA | HMDB00626 | Secondary | Unconjugated |
| 30E±,120E± | | | | |
| Dihydroxynorcholanate\ | NorDCA | NA | Secondary | Unconjugated |
| 23 Nordeoxycholic acid | | | | |
| Ursodeoxycholic Acid | UDCA | HMDB00946 | Secondary | Unconjugated |
| Lithocholic Acid | LCA | HMDB00761 | Secondary | Unconjugated |
| 6,7 diketolithocholic | 6,7_diketoLCA | NA | Secondary | Unconjugated |
| acid | | | | |
| Nutriacholic acid/7 | 7_ketoLCA | HMDB00467 | Secondary | Unconjugated |
| Ketolithocholic Acid | | | | |
| Lithocholic Acid 3 | LCA_3S | HMDB00907 | Secondary | Unconjugated |
| Sulfate | | | | |
| Hyodeoxycholic Acid | HDCA | HMDB00733 | Secondary | Unconjugated |
| Isolithocholic acid | isoLCA | HMDB00717 | Secondary | Unconjugated |
| βHyodeoxycholic Acid | βHDCA | HMDB00664 | Secondary | Unconjugated |
| (Isohyodeoxycholic acid) | | | | |
| Allolithocholic acid | alloLCA | HMDB00713 | Secondary | Unconjugated |
| Isoallolithocholic acid) | | | | |
| dehydroLCA | dehydroLCA | M07X058a_N2 | Secondary | Unconjugated |
| 12 Ketolithocholic | 12_ketoLCA | HMDB00328 | Secondary | Unconjugated |
| acid\12 | | | | |
| Ketodeoxycholic acid | | | | |
| 3β Ursodeoxycholic Acid | 3β UDCA | HMDB00686 | Secondary | Unconjugated |
| (Isoursodeoxycholic acid) | | | | |
| Glycodeoxycholic Acid | GDCA | HMDB00631 | Secondary | Conjugated |
| Tauroursodeoxycholic | TUDCA | HMDB00874 | Secondary | Conjugated |
| Acid | | | | |
| Glycoursodeoxycholic | GUDCA | HMDB00708 | Secondary | Conjugated |
| Acid | | | | |
| Taurodeoxycholic Acid | TDCA | HMDB00896 | Secondary | Conjugated |
| Glycolithocholic Acid 3 | GLCA_3S | HMDB02639 | Secondary | Conjugated |
| Sulfate | | | | |
| Glycolithocholate | GLCA | HMDB00698 | Secondary | Conjugated |
| Taurohyodeoxycholic Acid | THDCA | M07X076_N | Secondary | Conjugated |

| | | | | | Treatment | |
|---|---|---|---|---|---|---|
| | Population | | Remission | | Failure | |
| Metabolite | (N = 208) | | (N = 73) | | (N = 25) | |
| Cholic Acid | 268.79 | (26.61) | 223.02 | (25.21) | 189.76 | (32.02) |
| Chenodeoxycholic Acid | 417.81 | (36.10) | 433.58 | (47.12) | 200.08 | (23.28) |
| Chenodeoxycholic Acid | 285.92 | (21.28) | 331.33 | (36.98) | 257.71 | (53.79) |
| 24 Acyl β D | | | | | | |
| glucuronide | | | | | | |
| 3β Cholic Acid | 26.88 | (1.03) | 27.75 | (1.83) | 22.85 | (2.06) |
| Hyocholic Acid | 137.82 | (44.29) | 152.39 | (72.50) | 53.57 | (27.53) |
| ω Muricholic Acid | 70.65 | (2.29) | 64.87 | (3.08) | 62.97 | (5.64) |
| Nor Cholic acid | 26.03 | (2.01) | 24.38 | (2.97) | 23.71 | (5.47) |
| Taurocholic Acid | 119.14 | (18.02) | 109.20 | (14.50) | 121.85 | (40.14) |
| Glycocholic Acid | 485.04 | (45.56) | 522.85 | (68.65) | 388.60 | (88.50) |
| Taurochenodeoxycholate | 245.74 | (26.16) | 229.21 | (29.42) | 201.18 | (48.28) |
| Glycochenodeoxycholate | 1578.69 | (104.72) | 1775.25 | (168.81) | 1291.23 | (210.95) |
| 5β cholanic acid | | | | | | |
| 3β,7β,12α | | | | | | |
| triol 5β | | | | | | |
| Cholanic Acid | 25.03 | (1.66) | 24.12 | (2.66) | 22.54 | (3.48) |
| 3β,7β,12α | | | | | | |
| triol | | | | | | |
| Taurohyocholate | 52.68 | (4.72) | 53.51 | (8.65) | 34.80 | (6.56) |
| Glycohyocholate | 7.37 | (0.37) | 6.68 | (0.49) | 9.72 | (1.37) |
| Glycohyodeoxycholate | 6.42 | (0.40) | 6.53 | (0.62) | 5.41 | (0.92) |
| Deoxycholic Acid | 643.60 | (41.72) | 568.73 | (59.21) | 469.11 | (66.41) |
| 30E±,120E± | | | | | | |
| Dihydroxynorcholanate\ | 12.29 | (0.81) | 12.77 | (1.10) | 13.19 | (4.12) |
| 23 Nordeoxycholic acid | | | | | | |
| Ursodeoxycholic Acid | 124.34 | (14.83) | 163.84 | (39.10) | 82.60 | (14.42) |
| Lithocholic Acid | 21.51 | (1.74) | 18.22 | (1.61) | 17.09 | (2.73) |
| 6,7 diketolithocholic | 159.38 | (10.43) | 153.24 | (16.20) | 141.61 | (32.55) |
| acid | | | | | | |
| Nutriacholic acid/7 | 12.79 | (3.21) | 6.79 | (0.67) | 30.21 | (23.90) |
| Ketolithocholic Acid | | | | | | |
| Lithocholic Acid 3 | 21.86 | (0.91) | 19.51 | (1.02) | 19.31 | (2.29) |
| Sulfate | | | | | | |
| Hyodeoxycholic Acid | 92.22 | (7.96) | 97.19 | (14.07) | 65.02 | (13.37) |
| Isolithocholic acid | 38.32 | (2.58) | 41.33 | (5.24) | 22.98 | (5.87) |
| βHyodeoxycholic Acid | 25.30 | (2.62) | 22.60 | (4.36) | 21.25 | (4.89) |
| (Isohyodeoxycholic acid) | | | | | | |
| Allolithocholic acid | 36.49 | (2.42) | 33.85 | (4.01) | 27.46 | (5.97) |
| Isoallolithocholic acid) | | | | | | |
| dehydroLCA | 14.14 | (0.47) | 14.45 | (0.73) | 13.75 | (0.92) |
| 12 Ketolithocholic | 7.45 | (0.51) | 6.18 | (0.59) | 4.91 | (0.81) |
| acid\12 | | | | | | |
| Ketodeoxycholic acid | | | | | | |
| 3β Ursodeoxycholic Acid | 128.07 | (13.22) | 157.13 | (29.53) | 90.31 | (16.49) |
| (Isoursodeoxycholic acid) | | | | | | |
| Glycodeoxycholic Acid | 707.07 | (45.64) | 667.53 | (71.78) | 549.05 | (100.32) |
| Tauroursodeoxycholic | 20.42 | (1.21) | 22.85 | (2.13) | 24.49 | (5.12) |
| Acid | | | | | | |
| Glycoursodeoxycholic | 13.86 | (1.01) | 14.79 | (1.97) | 11.12 | (2.86) |
| Acid | | | | | | |
| Taurodeoxycholic Acid | 71.37 | (7.21) | 71.81 | (9.45) | 53.42 | (8.54) |
| Glycolithocholic Acid 3 | 800.54 | (74.44) | 650.96 | (73.37) | 788.42 | (219.88) |
| Sulfate | | | | | | |
| Glycolithocholate | 5.08 | (0.34) | 5.03 | (0.65) | 4.53 | (0.90) |
| Taurohyodeoxycholic Acid | 15.66 | (1.73) | 11.86 | (1.59) | 11.17 | (2.08) |

Abbreviations:
PReDICT: Predictors of Remission in Depression to Individual and Combined Treatments

### Primary BAs

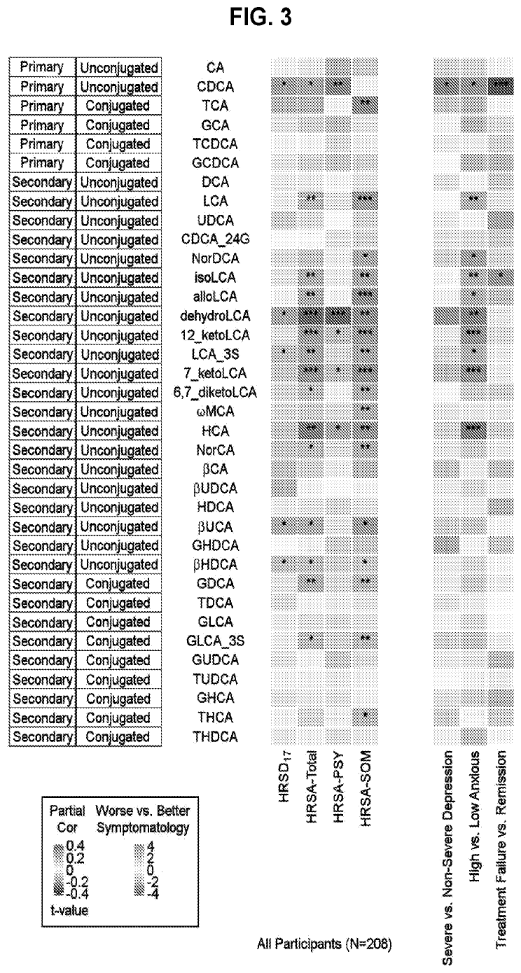

As depicted in FIG. 3, the primary bile acid CDCA, which is produced predominantly from the alternate pathway, was negatively correlated with the baseline total HRSD₁₇ score after adjusting for age, sex, and body mass index (partial correlation rho=−0.16, p=0.021). Dichotomous analysis showed a significantly lower CDCA in the more compared to the less severely depressed participants (p=0.02). No significant correlation or difference was noted for CA, the primary BA produced through the classical pathway (rho=−0.01, p=0.88; PWilcoxon=0.41).

### Secondary BAs

The secondary bacterially-produced BAs, lithocholic acid 3 sulfate (LCA_3 S) and isohyodeoxycholic acid (BHDCA) were positively correlated with HRSD₁₇ (rho=0.158, p=0.022 and rho=0.156, p=0.025, respectively) while dehydro-LCA was negatively correlated (rho=−0.154, p=0.027). Similar trends were noted in non-severe vs. severe depressed groups for the aforementioned analytes, but the differences did not reach the significance level.

### BA Profiles Related to Anxiety Symptom Severity

### Primary BAs

CDCA was negatively correlated with HRSA-Total (rho=−0.149, p=0.032) and HRSA-PSY (rho=−0.207, p=0.0028), but not HRSA-SOM (rho=−0.015, p=0.82). CDCA was significantly lower in the highly anxious participants (p=0.021). No significant correlation was noted for the other primary bile acid, CA (classical pathway). However, norcholic acid, which is a non-conjugated C23 homologue of the primary bile acid, CA, exhibited positive correlations with HRSA-Total (rho=0.163, p=0.019), and HRSA-SOM (rho=0.195, p=0.015).

### Secondary BAs

The bacterially derived 73-hydroxy epimer of CA, β-ursocholic acid and the CDCA-derived hyocholic acid were inversely correlated with HRSA-Total and HRSA-SOM (rho's range [−0.22 to −0.13], p's range [0.001 to 0.046]). LCA, produced by 7-alpha-dehydroxylation of CDCA, and several of its derivatives including 7-keto-LCA, isoLCA, alloLCA, and 12-ketoLCA, were strongly positively correlated with HRSA-Total and HRSA-SOM (rho's range [0.18-0.34], p's range [4.46×10⁻⁷ to 8.85×10⁻³]). These BAs were also significantly elevated or trended to be elevated in highly anxious compared to less anxious participants (p's between 0.0002-0.01). In contrast to LCA and many of its derivatives that correlated positively with anxiety severity, dehydroLCA (a known anti-inflammatory BA) was negatively correlated with HRSA-Total (rho=−0.266, p=0.0001), HRSA-SOM (rho=−0.195, p=0.004) and HRSA-PSY (rho=−0.266, p=0.0001). In addition, two secondary glycine conjugated BAs were positively correlated with HRSA-Total and HRSA-SOM scores: glycodeoxycholic acid (GDCA) (HRSA-Total: rho=0.20, p=0.002; HRSA-SOM: rho=0.18, p=0.006) and glycolithocholic acid 3 sulfate (GLCA_3 S) (HRSA-Total: rho=0.17, p=0.011; HRSA-SOM: rho=0.187, p=0.007).

Overall, greater baseline anxiety was associated with lower concentrations of the primary BAs (primarily CDCA) and their conjugated forms, and higher levels or concentrations of secondary BAs, derived from CDCA, such as the hepatotoxic LCA and many of its metabolites. The correlations between the BAs and HRSA-Total score were driven primarily by somatic anxiety symptoms.

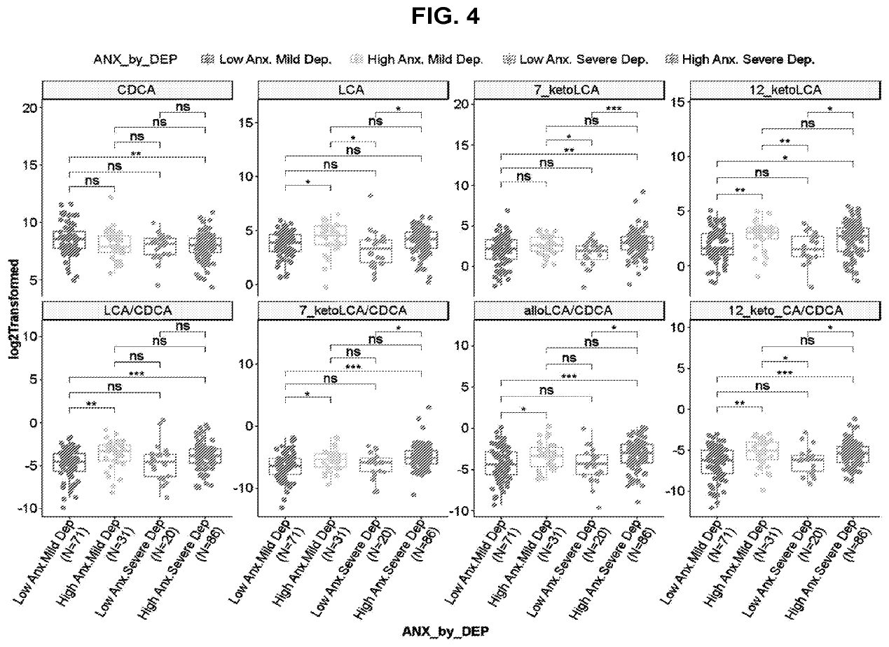

To investigate whether the differences observed in the BAs reported above were driven by anxiety or depression, the interaction effect of severity of anxiety and depression on the Bas was tested. As shown in FIG. 4, several gut-microbe-produced BAs and ratios of secondary to primary BAs (e.g., LCA, 7-ketoLCA, 12-ketoLCA, LCA/CDCA, 7-ketoLCA/CDCA, alloLCA/CDCA, 12-ketoLCA/CDCA) significantly differed between low versus highly anxious MDD participants irrespective of depression severity. For example, LCA levels were significantly higher in both non-severe depression-high anxiety and high depression-high anxiety participants compared to the non-severe depression-low anxiety and severe depression-low anxiety groups respectively (p=0.012 and p=0.016, respectively). This was also observed with the other CDCA derived BAs or the ratios (FIG. 4). These data suggest that the differences in these BA profiles are significantly associated with anxiety but not depressive symptom severity.

### Altered Metabolism of BAs Through Classical and Alternate Pathways in MDD Participants

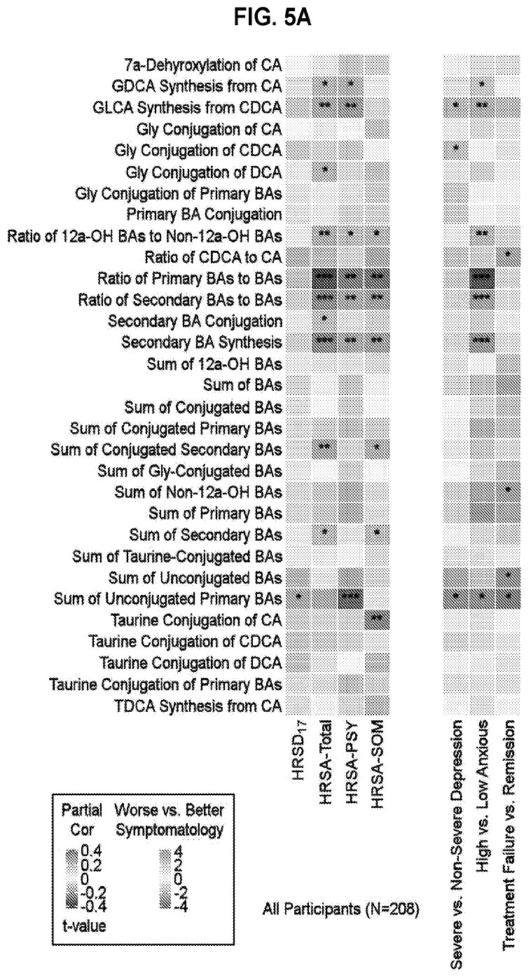

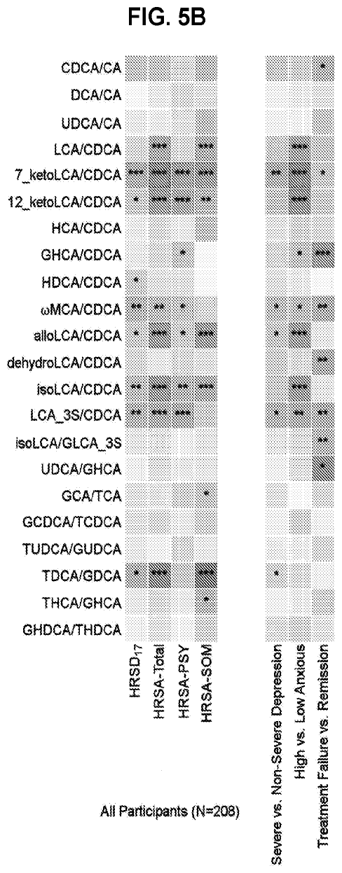

To investigate potential shifts in BA synthesis pathways or possible alterations in enzymatic activities, all possible pairwise BA ratios and selected composite summations and ratios were examined that could inform about changes in classical and alternate pathways of BA metabolism. A list of the BA summations and ratios and their implicated pathophysiology are shown in Table 3. Partial correlation analysis of depression severity score with composite summations and ratios did not yield strong correlation (FIG. 5A). However, a few ratios showed significant differences between patients with mild versus severe symptoms of anxiety. A higher value of the ratio of “primary BAs to total BAs,” which represents a fraction of primary BAs relative to the BA pool, was correlated to less severe anxiety. Concomitantly, lower values of the “secondary to primary BAs” ratio, which represents a fraction of secondary BAs relative to the BA pool, as well as “Secondary BA Synthesis”, which is the ratio of cytotoxic secondary BAs to primary BAs, were correlated with less severe anxiety symptomology (HRSA-Total). Both HRSA-PSY and HRSA-SOM were similarly affected (absolute rho's range [0.19 to 0.25], p's range [2.14×10⁻⁴ to 5.11×10⁻³]). Additionally, “sum of unconjugated primary BAs,” a higher level of which may indicate less BA conjugation and less solubility, was negatively correlated with HRSA-PSY (rho=−0.22, p=9.61×10⁻⁴).

### PLS Analyses

VIP scores from PLS analyses are presented in Table 3. The PLS regression model with HRSD₁₇ as the dependent variable resulted in six significant BAs (VIP score >1), and four primary (including both conjugated and non-conjugated) and two secondary BAs as important contributors to depression severity. These were glycolithocholic acid-3 sulfate (GLCA_3 S) (VIP=3.12), chenodeoxycholic acid (CDCA) (VIP=2.95), glycodeoxycholic acid (GCDCA) (VIP=2.84), glycocholic acid (GCA) (VIP=1.86), taurocholic acid (TCA) (VIP=1.31), and ursodeoxycholic acid (UDCA) (VIP=1.21).

The PLS regression model with HRSA-Total score as the dependent variable resulted in three primary and two secondary BAs as important contributors to anxiety symptoms. These were GLCA_3 s (VIP=3.28), glycodeoxycholic acid (GDCA) (VIP=3.06), GCDCA (VIP=3.05), GCA (VIP=1.28), CDCA (VIP=1.24) and CDCA_24 G (VIP=1.11).

A PLSDA was also conducted to investigate which BAs could most accurately distinguish between remitters and treatment failures at baseline. The first component of the PLSDA model achieved an area under the curve of 0.7923 (p=0.00002). Based on the VIP scores, CDCA (more abundant in remitters) was the most important variable in discriminating remitters from those with treatment failure.

TABLE 3
Bile Acid Ratios, Summations, and their Association with Metabolic Reactions and Dysfunction.

| Ratios indicating metabolic | | | Findings in |
|---|---|---|---|
| reactions in BA pathway | Formula | Description | PReDICT |
| 7a-Dehydroxylation of CA | DCA/CA | | |
| | Conversion of the primary | N.S | |
| | bile acid CA to the | | |
| | secondary bile acid DCA | | |
| | by gut bacteria. The ratio | | |
| | is strongly associated with | | |
| | a cognitive decline. | | |
| GDCA Synthesis from CA | GDCA/CA | | |
| | Conversion of the primary | Higher ratio values | |
| | bile acid CA to the | correlate with higher | |
| | conjugated secondary bile | HRSA-Total scores | |
| | acid GDCA involving the | | |
| | gut microbiota. The ratio | | |
| | is strongly associated with | | |
| | cognitive decline. | | |
| GLCA Synthesis from CDCA | GLCA/CDCA | Higher ratio values | |
| | Conversion of the primary | correlate with higher | |
| | bile acid CDCA to the | HRSA-Total scores | |
| | conjugated secondary bile | | |
| | acid GLCA involving the | | |
| | gut microbiota. The ratio | | |
| | is strongly associated with | | |
| | cognitive decline. | | |
| Gly Conjugation of CA | GCA/CA | | |
| | Indicator of conjugation of | N.S | |
| | glycine to the primary bile | | |
| | acid CA to form GCA. | | |
| Gly Conjugation of CDCA | GCDCA/CDCA | Indicator of | N.S |
| | | conjugation of | |
| | | glycine to the | |
| | | primary bile acid | |
| | | CDCA to form | |
| | | GCDCA. | |
| Gly Conjugation of DCA | GDCA/DCA | Indicator of | Higher ratio |
| | | conjugation of | values correlate |
| | | glycine to the | with higher |
| | | secondary bile acid | HRSA-Total |
| | | DCA to form GDCA. | scores |
| Gly Conjugation of Primary | (GCA + GCDCA)/(CA + | Indicator of | N.S |
| BAs | CDCA) | conjugation of | |
| | | glycine to the | |
| | | primary bile acids | |
| | | CA and CDCA to | |
| | | form the glycine- | |
| | | conjugated primary | |
| | | bile acids GCA and | |
| | | GCDCA. | |
| Primary BA Conjugation | (GCA + GCDCA + TCA + | Indicator of | N.S |
| | TCDCA)/(CA + CDCA) | conjugation of | |
| | | glycine or taurine to | |
| | | primary BAs to form | |
| | | conjugated primary | |
| | | BAs in the liver. | |
| Ratio of 12a-OH BAs to Non- | (CA + DCA + GCA + | | |
| 12a-OH BAs | GDCA + TCA+ | | |
| TDCA)/(CDCA + GCDCA + | | | |
| GLCA+ | | | |
| GUDCA + TCDCA + TLCA) | The ratio of 12-alpha- | Higher ratio values | |
| | hydroxylated BAs to non- | correlate with higher | |
| | 12-alpha-hydroxylated | HRSA-Total scores | |
| | BAs is an indicator of type | | |
| | 2 diabetes. | | |
| Ratio of CDCA to CA | CDCA/CA | Ratio of the two | N.S |
| | | primary BAs. CA is | |
| | | synthesized from | |
| | | cholesterol in the | |
| | | classical pathway, | |
| | | while CDCA | |
| | | primarily comes | |
| | | from the alternative | |
| | | pathway. | |
| Ratio of Primary BAs to BAs | (CA + CDCA + GCA + | | |
| | GCDCA + TCA + TCDCA)/ | | |
| | (CA + CDCA + DCA + | | |
| | GCA + GCDCA + GDCA + | | |
| | GLCA + GUDCA+ | | |
| TCA + TCDCA + TDCA + | Fraction of primary BAs | Higher ratio values | |
| TLCA) | relative to the BA pool. | correlate with lower | |
| | Primary BAs are | HRSA-Total scores | |
| | synthesized from | | |
| | cholesterol in the liver, | | |
| | conjugated with either | | |
| | taurine or glycine, and | | |
| | then released into the | | |
| | biliary system. After their | | |
| | excretion into the | | |
| | gastrointestinal tract, some | | |
| | of them are deconjugated | | |
| | and modified by bacterial | | |
| | metabolism, leading to | | |
| | secondary BAs. | | |
| Ratio of Secondary BAs to | (DCA + GDCA + GLCA + | | |
| BAs | GUDCA+ | | |
| TDCA + TLCA)/(CA + CDCA + | | | |
| DCA + | | | |
| GCA + GCDCA + GDCA + | | | |
| GLCA+ | | | |
| GUDCA + TCA + TCDCA + | Fraction of secondary BAs | Higher ratio values | |
| TDCA + TLCA) | relative to the BA pool. | correlate with higher | |
| | | HRSA-Total scores | |
| Secondary BA Conjugation | (GDCA + GLCA + GUDCA + | | |
| TLCA)/DCA | TDCA+ | | |
| | Indicator of conjugation of | Higher ratio values | |
| | glycine or taurine to | weakly correlates | |
| | secondary BAs to form | with higher HRSA- | |
| | conjugated secondary BAs | Total scores | |
| | in the liver. | | |
| Secondary BA Synthesis | (DCA + GDCA + GLCA + | | |
| | GUDCA+ | | |
| TDCA + TLCA)/(CA + CDCA + | Ratio of cytotoxic | Higher ratio values | |
| GCA + GCDCA + TCA + | secondary BAs to primary | correlate with higher | |
| TCDCA) | BAs. Secondary BAs | HRSA-Total scores | |
| | solely produced by | | |
| | intestinal bacteria can | | |
| | accumulate to a high | | |
| | degree in the | | |
| | enterohepatic circulation of | | |
| | some individuals and may | | |
| | contribute to the | | |
| | pathogenesis of colon | | |
| | cancer, gallstones, and | | |
| | other gastrointestinal | | |
| | diseases. | | |
| Sum of 12a-OH BAs | CA + DCA + GCA + | | |
| | GDCA + TCA + TDCA | | |
| | It has been shown that | N.S | |
| | increased levels of 12- | | |
| | alpha-hydroxylated BAs | | |
| | are associated with insulin | | |
| | resistance. | | |
| Sum of BAs | CA + CDCA + DCA + GCA + | | |
| | GCDCA + GDCA + | | |
| | GLCA + GUDCA + TCA+ | | |
| TCDCA + TDCA + TLCA | Sum of BAs is considered | N.S | |
| | to be a biomarker of liver | | |
| | function. | | |
| Sum of Conjugated BAs | GCA + GCDCA + GDCA + | | |
| | GLCA+ | | |
| GUDCA + TCA + TCDCA + | Conjugation with Gly or | N.S | |
| TDCA + TLCA | Tau increase solubility of | | |
| | the BAs and make them | | |
| | impermeable to cell | | |
| | membranes. Conjugated | | |
| | BAs may be increased in | | |
| | plasma due to mutations | | |
| | or antibiotic treatments. | | |
| Sum of Conjugated Primary | GCA + GCDCA + TCA + | Conjugated primary | N.S |
| BAs | TCDCA | BAs are significantly | |
| | | elevated in patients | |
| | | with polycystic ovary | |
| | | syndrome. In | |
| | | addition, conjugated | |
| | | primary BAs were | |
| | | found to be elevated | |
| | | after antibiotic | |
| | | treatment. | |
| Sum of Conjugated Secondary | GDCA + GLCA + GUDCA + | Concentrations of | Higher ratio |
| BAs | TDCA + TLCA | conjugated | values correlate |
| | | secondary BAs may | with higher |
| | | be reduced after | HRSA-Total |
| | | antibiotic treatment. | scores |
| Sum of Gly-Conjugated BAs | GCA + GCDCA + GDCA + | The content of | N.S |
| | GLCA + GUDCA | taurine-conjugated | |
| | | BAs normally | |
| | | correlates with the | |
| | | content of glycine- | |
| | | conjugated BAs, | |
| | | unless the levels of | |
| | | taurine or glycine | |
| | | are abnormal. | |
| Sum of Non-12a-OH BAs | CDCA + GCDCA + GLCA + | Sum of non-12- | N.S |
| | GUDCA + TCDCA + | alpha-hydroxylated | |
| | TLCA | BAs. | |
| Sum of Primary BAs | CA + CDCA + GCA + | Elevated primary BA | N.S |
| | GCDCA + TCA + TCDCA | levels in plasma or | |
| | | serum are often | |
| | | caused by biliary | |
| | | obstruction, e.g., in | |
| | | cholelithiasis | |
| | | (gallstones), | |
| | | pancreatitis, or | |
| | | pancreatic cancer. | |
| Sum of Secondary BAs | DCA + GDCA + GLCA + | | |
| | GUDCA+ | | |
| TDCA + TLCA | | | |
| | High fat and high beef | Higher ratio values | |
| | diets promote bile | correlate with higher | |
| | discharge and influence | HRSA-Total scores | |
| | the bacterial composition | | |
| | in the gut, resulting in | | |
| | increased levels of | | |
| | secondary BAs associated | | |
| | with an increased risk for | | |
| | colon cancer. Low levels | | |
| | of secondary BAs may | | |
| | indicate a reduced | | |
| | capacity of the gut | | |
| | microbiota to metabolize | | |
| | primary BAs. | | |
| Sum of Taurine-Conjugated | TCA + TCDCA + TDCA + | The content of | N.S |
| BAs | TLCA | taurine-conjugated | |
| | | BAs normally | |
| | | correlates with the | |
| | | content of glycine- | |
| | | conjugated BAs, | |
| | | unless the levels of | |
| | | taurine or glycine | |
| | | are abnormal. | |
| Sum of Unconjugated BAs | CA + CDCA + DCA | | |
| | BAs are synthesized in the | N.S | |
| | liver and then usually | | |
| | conjugated with taurine or | | |
| | glycine to increase | | |
| | solubility and make them | | |
| | impermeable to cell | | |
| | membranes, only a | | |
| | fraction of BAs remains | | |
| | unconjugated. | | |
| | Unconjugated BAs may be | | |
| | increased in several | | |
| | hepatobiliary diseases, | | |
| | e.g., cholangiocarcinoma | | |
| | and hepatocellular | | |
| | carcinoma. | | |
| Sum of Unconjugated Primary | CA + CDCA | | |
| BAs | | | |
| | Unconjugated primary BAs | Higher ratio values | |
| | are produced from | correlate with lower | |
| | cholesterol in the liver and | HRSA-Total scores | |
| | are less hydrophilic than | | |
| | upon conjugation to | | |
| | glycine or taurine to | | |
| | increase solubility. | | |
| | Elevated primary BA levels | | |
| | in plasma or serum are | | |
| | often caused by biliary | | |
| | obstruction, e.g., in | | |
| | cholelithiasis (gallstones), | | |
| | pancreatitis, or pancreatic | | |
| | cancer. | | |
| Taurine Conjugation of CA | TCA/CA | The ratio of the | Higher ratio |
| | | taurine-conjugated | values correlate |
| | | primary BA | with higher |
| | | taurocholic acid to | HRSA-SOM |
| | | the unconjugated | scores |
| | | primary BA cholic | |
| | | acid is an indicator | |
| | | of bile acid CoA | |
| | | ligase and bile acid | |
| | | CoA:amino acid N- | |
| | | acyltransferase | |
| | | activity in the liver. | |
| Taurine Conjugation of CDCA | TCDCA/CDCA | The ratio of the | N.S |
| | | taurine-conjugated | |
| | | primary BA | |
| | | taurochenodeoxycholic | |
| | | acid to the | |
| | | unconjugated | |
| | | primary BA | |
| | | chenodeoxycholic | |
| | | acid is an indicator | |
| | | of bile acid CoA | |
| | | ligase and bile acid | |
| | | CoA:amino acid N- | |
| | | acyltransferase | |
| | | activity in the liver. | |
| Taurine Conjugation of DCA | TDCA/DCA | The ratio of the | N.S |
| | | taurine-conjugated | |
| | | secondary BA | |
| | | taurodeoxycholic | |
| | | acid to the | |
| | | unconjugated | |
| | | secondary BA | |
| | | deoxycholic acid is | |
| | | an indicator of bile | |
| | | acid CoA ligase and | |
| | | bile acid CoA:amino | |
| | | acid N- | |
| | | acyltransferase | |
| | | activity in the liver. | |
| Taurine Conjugation of | (TCA + TCDCA)/(CA + | The ratio of the | N.S |
| Primary BAs | CDCA) | taurine-conjugated | |
| | | primary BAs | |
| | | taurocholic acid and | |
| | | taurochenodeoxych | |
| | | olic acid to the | |
| | | unconjugated | |
| | | primary BAs cholic | |
| | | acid and | |
| | | chenodeoxycholic | |
| | | acid is an indicator | |
| | | of bile acid CoA | |
| | | ligase and bile acid | |
| | | CoA:amino acid N- | |
| | | acyltransferase | |
| | | activity in the liver. | |
| TDCA Synthesis from CA | TDCA/CA | | |
| | Ratio of the taurine- | N.S | |
| | conjugated secondary BA | | |
| | taurodeoxycholic acid to | | |
| | the unconjugated primary | | |
| | BA cholic acid, which is | | |
| | strongly associated with | | |
| | cognitive decline. | | |

Abbreviations: BA: Bile Acid; CA: Cholic Acid; CDCA: Chenodeoxycholic Acid; DCA: Deoxycholic Acid; GCA: Glycocholic Acid; GCDCA: Glycodeoxycholic Acid; GDCA: Glycodeoxycholate; GLCA: Glycolithocholate; GUDCA: Glycoursodeoxycholic Acid; HRSA-Total: Clinician-rated 14-item Hamilton Anxiety Rating Scale; N.S.: Not Significant; TCA: Taurocholic Acid; TCDCA: Taurochenodeoxycholic Acid; TLCA: Taurolithocholic Acid.

Ratios of CDCA/CA, which is an indicator of a shift in BA synthesis from classical to alternate pathway, as well as conjugated/unconjugated BA ratio for the taurine or glycine conjugations, did not yield significant correlations.

In high anxiety vs. low anxiety patients, the most significant differences in pairwise ratios were observed in the ratios of secondary to the (precursor) primary CDCA such as LCA/CDCA (p=0.0001), 7-ketoLCA/CDCA (p=6.85×10⁻⁶), 12-ketoLCA/CDCA (p=4.87×10⁻⁵), alloLCA/CDCA (p=0.0001), isoLCA/CDCA (p=3.57×10⁻⁵), LCA-3S/CDCA (p=0.002), glycohyocholic acid (GHCA)/CDCA (p=0.041), omega monocarboxylic acid (wMCA)/CDCA (p=0.021), all of which were significantly higher in patients with more severe symptoms, particularly HRSA-PSY. This suggests an increased utilization of CDCA for the synthesis of bacterially-derived secondary BA in these participants (FIG. 5B).

Partial correlation analysis of BA ratios and anxiety scores also showed that the gut-bacteria that was produced secondary BAs to their precursor primary BA ratios such as LCA/CDCA, 7-ketoLCA/CDCA, 12-ketoLCA/CDCA, alloLCA/CDCA, isoLCA/CDCA, LCA-3S/CDCA were significantly positively correlated with anxiety symptoms (rho's range [0.14 to 0.35], p's range [2.32×10⁻⁷ to 4.16×10⁻²]). The ratio of the taurine to glycine conjugated deoxycholic acid, TDCA/GDCA, was significantly negatively correlated to HRSA-SOM (rho=−0.27; p=7.22×10⁻⁵). Thus overall, the ratio data indicate a significant trend towards higher levels of secondary BAs compared to their primary precursors that correlated with more anxiety severity in these MDD participants, which suggests gut microbiome dysbiosis in more anxious patients.

Baseline BA Concentrations Distinguish Participants Who Reached Symptom Remission from Those Who Experienced Treatment Failure from 12 Weeks of Treatment

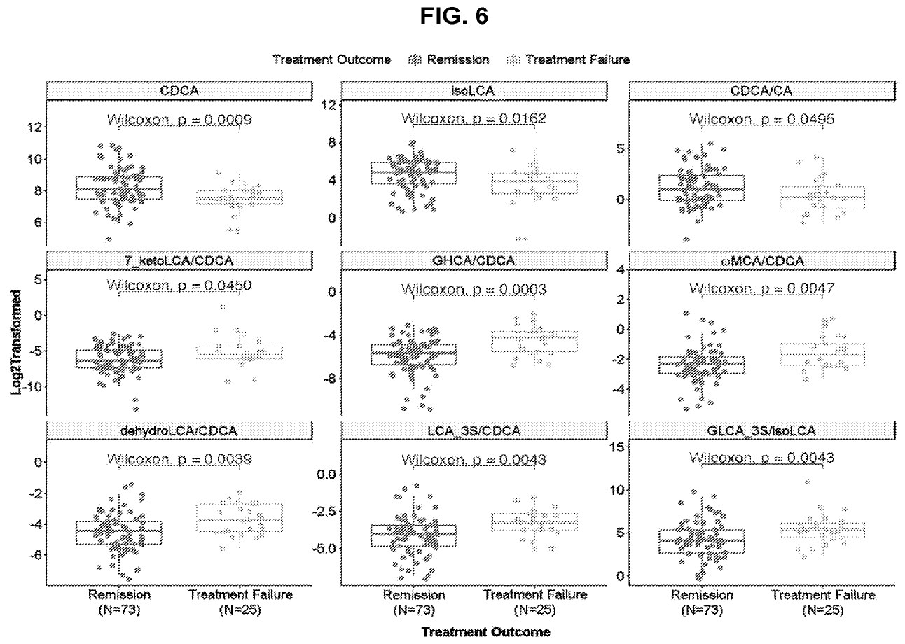

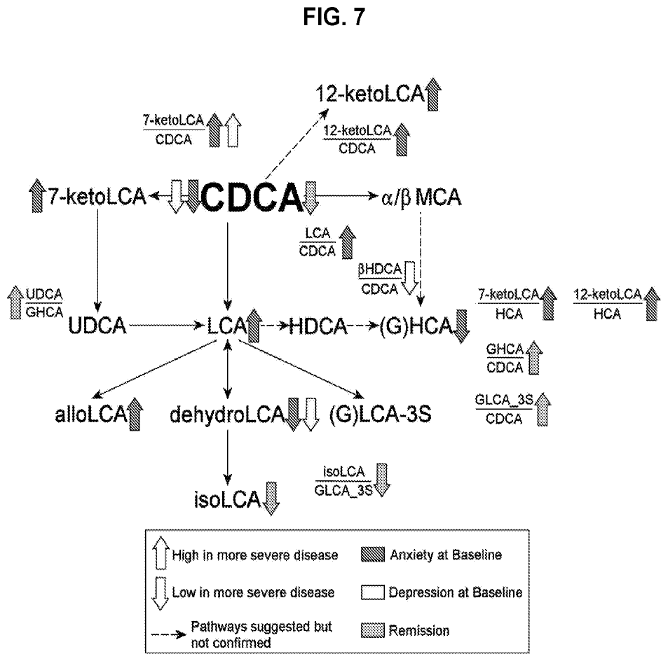

The study examined whether any of the metabolites that were associated with depression and/or anxiety symptom severity at baseline were different in participants who responded to treatment (remitters; N=73) versus those who did not respond to treatment (treatment failures; N=25) after 12 weeks of treatment/therapy. The metabolites which showed significantly higher baseline levels (p<0.05) in remitters compared to the treatment failures were the primary bile acid, CDCA (p=0.0009), its bacterial derivative isoLCA (p=0.0162) (FIGS. 3 and 6) and the ratio of the two primary bile acids CDCA/CA (p=0.0495) (FIGS. 5B and 6). Several secondary BA to CDCA ratios such as 7-ketoLCA/CDCA, GHCA/CDCA, ωMCA/CDCA, dehydroLCA/CDCA, LCA-3S/CDCA and the secondary to secondary ratio, GLCA-3S/isoLCA (FIGS. 5B and 6) were significantly lower at baseline in the remitters compared to the treatment failures (p's range [0.00032-0.0495]).

TABLE 4
Partial Least Squares Discriminant Analysis Scores for Contribution
of Bile Acids to Severity of Clinical Symptoms.

| | Anxiety | Depression | Treatment Outcome |
|---|---|---|---|
| Metabolite | VIP Score | VIP Score | VIP Score |
| GLCA_3S | 3.28 | 3.12 | 0.49 |
| GDCA | 3.06 | 0.95 | 0.94 |
| GCDCA | 3.05 | 2.84 | 1.35 |
| GCA | 1.28 | 1.86 | 0.93 |
| CDCA | 1.24 | 2.95 | 2.45 |
| CDCA_24G | 1.11 | 0 | 0.87 |
| TCA | 0.74 | 1.31 | 0.6 |
| CA | 0.64 | 0.26 | 0.81 |
| TCDCA | 0.5 | 0.66 | 0.18 |
| 6,7_diketoLCA | 0.45 | 0.32 | 0.17 |
| UDCA | 0.38 | 1.21 | 1.05 |
| DCA | 0.36 | 0.76 | 1.19 |
| HCA | 0.36 | 0.52 | 0.8 |
| βHDCA | 0.32 | 0.29 | 0.37 |
| TDCA | 0.22 | 0.22 | 1.01 |
| isoLCA | 0.18 | 0.14 | 1.71 |
| THCA | 0.17 | 0.13 | 1.24 |
| NorCA | 0.17 | 0.12 | 0.15 |
| βUDCA | 0.15 | 0.55 | 1.21 |
| alloLCA | 0.12 | 0.03 | 0.68 |
| ωMCA | 0.11 | 0.14 | 0.38 |
| βUCA | 0.08 | 0.1 | 0.12 |
| 7_ketoLCA | 0.07 | 0.08 | 1.37 |
| HDCA | 0.06 | 0.14 | 1.17 |
| LCA_3S | 0.06 | 0.06 | 0.1 |
| 12_ketoLCA | 0.04 | 0.02 | 1.2 |
| GUDCA | 0.04 | 0.04 | 0.93 |
| LCA | 0.04 | 0.02 | 0.22 |
| GHDCA | 0.03 | 0.02 | 0.62 |
| dehydroLCA | 0.03 | 0.03 | 0.53 |
| TUDCA | 0.03 | 0.04 | 0.42 |
| THDCA | 0.03 | 0 | 0.28 |
| NorDCA | 0.03 | 0.01 | 0.03 |
| GHCA | 0.01 | 0.02 | 2.19 |
| βCA | 0.01 | 0.06 | 1.45 |
| GLCA | 0 | 0.02 | 0.14 |

Abbreviations: CA: Cholic Acid; alloLCA: Allolithocholic Acid; CDCA: Chenodeoxycholic Acid; DCA: Deoxycholic Acid; GCA: Glycocholic Acid; GCDCA: Glycodeoxycholic Acid; GDCA: Glycodeoxycholic Acid; GHCA: Glycohyocholic Acid; GHDCA: Glycohyodeoxycholic Acid; GLCA: Glycolithocholic Acid; GUDCA: Glycoursodeoxycholic Acid; HCA: Hydroxycitric Acid; HDCA: Hyodeoxycholic Acid; isoLCA: Isolithocholic Acid; LCA: Lithocholic Acid; MCA: Monocarboxylic Acid; NorCA: Norcholic Acid; TCA: Taurocholic Acid; TCDCA: Taurochenodeoxycholic Acid; TDCA: Taurodeoxycholic Acid; THCA: Tetrahydrocannabinolic Acid; THDCA: Taurohyodeoxycholic Acid; TUDCA: Tauroursodeoxycholic Acid; UCA: Ursocholic Acid; UDCA: Ursodeoxycholic Acid; VIP: Variable Importance on Projection.

Mounting evidence indicates that gut dysbiosis and the bidirectional communication between brain and gut microflora play an important role in the development of neuropsychiatric diseases. Using targeted metabolomics in participants with MDD, this study showed that increased levels of cytotoxic secondary BAs, bacterially-derived from the primary bile acid CDCA, correlated with anxiety symptom severity. The classical pathway that, predominantly, produces the primary bile acid CA seemed to be less impacted. Additionally, participants who did not to benefit from treatment were found to have higher baseline levels of the cytotoxic secondary BAs derived from CDCA. These findings suggest that alternate therapies might be needed that target the gut microbiome for patients who have gut dysbiosis.

The study addressed whether BA concentrations impacted depression and anxiety symptom severity. Overall, BA concentrations appeared to have a stronger impact on anxiety than on depression. Several secondary BA concentrations, and the ratios of secondary to primary BAs, were significantly different between low versus high-anxious MDD participants irrespective of depression severity. These secondary BAs included LCA and its derivatives, 7-keto-LCA, isoLCA, alloLCA and 12-ketoLCA. The 7α-dehydroxylation reaction that results in the formation of the secondary BAs has been described as the most quantitatively important process performed by colonic bacteria belonging to the genus Clostridium, an enzymatic reaction that is impacted in many neurological diseases. LCA is produced by 7α-dehydroxylation of CDCA and is known to be cytotoxic in rodents as well as several human cell types.

The study addressed whether there were any associations of symptoms with the classical and alternate pathways of BA synthesis. In Alzheimer's disease, a significant shift in BA synthesis from classical to the alternative pathways was observed in the Alzheimer's participants compared to healthy controls. In these MDD participants, the alternate pathway that favors CDCA synthesis was significantly impacted in the highly-anxious participants. However, no shift from classical to alternate pathway could be observed in these participants since the ratio of CA/CDCA, which indicates such a shift, was not significantly associated with symptom severity. Lower CDCA levels and higher secondary metabolites derived from CDCA (and mostly higher ratios of these secondary BAs to CDCA) characterized the participants with higher symptom severity, which may indicate greater utilization of CDCA by the gut bacteria. There was no significant impact of glycine and taurine conjugation of BA on symptom severity. Interestingly, dehydrolithocholic acid, a major metabolite of LCA, was strongly negatively correlated to anxiety levels in the MDD participants. It is an agonist of the nuclear receptors GPCR1, the farnesoid X receptor (FXR) and the pregnane X receptor and has recently been shown to regulate adaptive immunity by inhibiting the differentiation of TH17 cells that are known to cause autoimmunity and inflammation. The study also examined whether any relationship exists between baseline metabolite levels and response to treatment. Remitters showed higher levels of CDCA and one of its gut microbial metabolites (isoLCA) compared to participants for whom the treatment failed.

The enzymatic processes involved in altered BA metabolism in CNS diseases may be informed by the association of BAs with inborn errors of metabolism (IEM), in which reduced intestinal BA concentrations result in serious morbidity or mortality. To date, nine recognized inborn errors of BA metabolism have been identified that lead to enzyme deficiencies and impaired BA synthesis. These diseases are characterized by a failure to produce primary BAS and an accumulation of unusual BAs and BA intermediaries. Administration of BAs for replacement therapy often improves the symptoms of IEM, such as cerebrotendinous xanthomatosis, with CDCA the predominant choice for treating both neurological and non-neurological symptoms. A common link between IEM and depression through acylcarnitines and beta oxidation of fatty acids, in which medium-chain acyl-coenzyme A dehydrogenase, an enzyme involved in the production of medium chain acylcarnitines, was shown to be causally linked to depression and also to IEM. These emerging data linking metabolomic disturbances in CNS disorders and IEM provide novel insights into pathobiological processes that contribute to psychiatric disorders.

BAs influence metabolic processes by acting as signaling molecules via the nuclear receptors FXR, the pregnane X receptor, the vitamin D receptor, Takeda G-protein-coupled bile acid receptor, and sphingosine-1-phosphate receptor 2, initiating a variety of signaling cascades relevant to metabolic and hepatic diseases such as obesity, steatosis and steatohepatitis, as well as liver and colon cancer. FXR plays many important roles in the regulation mechanisms of BA synthesis and transport. FXR activation represses the expression of the main enzymes in BA synthesis, CYP7A1 and CYP27A1. In contrast, FXR activation upregulates UGT2B4, which is involved in the conversion of hydrophobic BAs to their less toxic glucuronide derivatives. CDCA is the most potent activator of FXR. Studies in knockout mice suggest the involvement of FXR in modulating brain function. Deletion of FXR altered the levels of several neurotransmitters in the hippocampus and cerebellum, and impaired cognitive function and motor coordination, which suggests that FXR signaling is required for normal brain function. A study using a rat-model found that over-expression of hippocampal FXR-mediated chronic unpredictable stress-induced depression-like behaviors and decreased hippocampal brain-derived neurotrophic factor expression, and that knocking out of hippocampal FXR completely prevented depressive behaviors via brain-derived neurotrophic factor expression.

LCA is the most potent ligand for Takeda G-protein-coupled BA receptor, and BA-dependent Takeda G-protein-coupled BA receptor-mediated signaling has been shown to influence the brain by regulating the production of the gut peptide hormone GLP-1, which potentiates glucose-stimulated insulin secretion. LCA is also a potent activator of pregnane X receptor and vitamin D receptor. Thus, largely through their binding and activation of these receptors, BAs regulate their own synthesis, conjugation, transport, and detoxification, as well as lipid, glucose, and energy homeostasis.

Of particular pathognomonic significance in MDD patients is probably the decrease in CDCA with concomitant increase in LCA, the latter being formed in humans mainly from the intestinal bacterial 7α-dehydroxylation of CDCA. LCA comprises less than 5% of the total BA pool in humans and is one of the most hydrophobic naturally occurring BAs.

LCA has been shown to induce double-strand breaks in DNA. The mammalian host responds by metabolizing LCA, mainly through sulfation, enabling more efficient excretion and reduced hydrophobicity. BA sulfation is an important detoxification process that converts hydrophobic BAs into excretable metabolites in the liver. Sulfation is catalyzed by a group of enzymes called sulfotransferases. Although, only a small proportion of BAs in bile and serum are sulfated, more than 70% of BAs in urine are sulfated, indicating their efficient elimination in urine. It is estimated that 40-75% of the hydrophobic, hepatotoxic LCA in human bile is present in the sulfated form. The formation of BA-sulfates increases during cholestatic diseases. Therefore, sulfation may play an important role in maintaining BA homeostasis under pathologic conditions. In this study, elevated levels of the sulfated form of the toxic LCA and GLCA were observed in more severely anxious patients. Previous studies showed increased production of other bacterially derived sulfates like p-cresol sulfate and indoxyl sulfates in the PREDICT study participants. Together, these may suggest that alterations in sulfotransferase activities may occur in the liver of some patients.

The microbial conversion of CDCA to 7-keto-LCA, which were observed to be present at higher levels in highly-anxious MDD participants, is known to be reduced in the liver by human 11β-HSDH-1, an enzyme with the primary function of converting cortisone to the active glucocorticoid, cortisol. Microbial-derived 7-keto-LCA acts as a competitive inhibitor of 11β-HSDH-1, and thus may influence the ratio of cortisone/cortisol.

There are a few limitations to this study. First, it lacked a healthy control group to compare with the participants who had MDD. Second, the study did not apply multiple comparison adjustments due to the relatively small sample size and the exploratory nature of this study. Third, the findings should be replicated in an independent cohort. Fourth, a number of novel BAs have recently been discovered and were not included in the metabolomic analyses; these compounds should be evaluated in future studies.

It has been suggested that in the highly evolutionary competitive environment of the human gut microbiome, the persistence of these microbial enzyme activities usually indicates that they increase the organism's ability to survive. However, dysbiosis in the gut is also possible. These data suggest that low levels of CDCA might be a result of increased utilization for production of bacterial products in the intestine which, in turn, suggest gut-microbe composition changes or associated enzymatic changes. The underlying pathophysiological significance of BA pool changes remain to be determined, but a reasonable hypothesis emerging from this work is that increases in circulating BAs result from a more hydrophobic BA pool in the colon resulting from gut microbial dysbiosis. These BAs may then produce enhanced toxicity and pathophysiology to cells in the liver, gastrointestinal tract, and the brain.

1-5. (canceled)

6. A method for stratifying and treating a subject having a depression, anxiety, mood disorders, or neuropsychiatric symptoms, or at risk of developing a neurological disorder, based on the subject's metabolic profile, the method comprising:
analyzing a sample from a subject to determine concentration levels or ratios of one or more biomarker metabolites or gut microbiome-related biomarker metabolites related to bile acid anabolism, catabolism or homeostasis in the sample from the subject;
determining if the subject has a metabolic defect related to disrupted bile acid anabolism, catabolism or homeostasis, or if the subject's gut microbiome has a defect related to disrupted bile acid anabolism, catabolism or homeostasis, or combinations thereof based on the measured concentration levels and calculated ratios of the one or more bile acid anabolism, catabolism or homeostasis biomarker metabolites in the sample as compared to a control sample;
stratifying the subject into a subgroup of subjects, wherein an individual subgroup of subjects is defined by a unique and specific bile acid anabolism, catabolism or homeostasis profile based on the measured concentration levels and calculated ratios of the one or more biomarker metabolites or gut microbiome-related biomarker metabolites in the sample as compared to a control sample and the biomarker metabolite or gut microbiome-related biomarker metabolite defect determined for the subject.

7. The method of claim 6, further comprising treating the depression, anxiety, mood disorders, or neuropsychiatric symptoms by administering to the subgroup of subjects an effective amount sufficient to attenuate, reduce, or eliminate the symptoms of depression, anxiety, mood disorders, or neuropsychiatric symptoms of a therapy to wherein the therapy is determined by the unique and specific metabolic profile of the subgroup of subjects.

8. The method of claim 6, wherein the one or more biomarker metabolites or gut microbiome-related biomarker metabolites comprises one or more of:
Primary Bile Acids: cholic acid (CA); chenodeoxycholic acid (CDCA); chenodeoxycholic acid-24-acyl-β-d-glucuronide (CDCA-24G); 3β-cholic acid (β-CA); hyocholic acid (HCA); ω-monocarboxylic acid (ω-MCA); taurocholic acid (TCA); glycocholic acid (GCA); taurochenodeoxycholate (TCDCA); glycochenodeoxycholate (GCDCA); 5β-cholanic acid-3β,7β,12α-triol-5β-cholanic acid-3β, 7β, 12α-triol (β-UCA); taurohyocholate (THCA); glycohyocholate (GHCA);
Secondary Bile Acids: glycohyodeoxycholate (GHDCA); deoxycholic acid (DCA); 23-nordeoxycholic acid (NorDCA); β-ursodeoxycholic acid (UDCA); lithocholic acid (LCA); 6,7-diketolithocholic acid (6,7-diketoLCA); 7-ketolithocholic acid (7-ketoLCA); lithocholic acid-3-sulfate (LCA-3S); hyodeoxycholic acid (HDCA); isolithocholic acid (isoLCA); β-hyodeoxycholic acid (isohyodeoxycholic acid; β-HDCA); allolithocholic acid (isoallolithocholic acid; alloLCA); dehydroLCA; 12-ketodeoxycholic acid (12-ketoLCA); 3β-ursodeoxycholic acid (isoursodeoxycholic acid; 3β-UDCA); glycodeoxycholic acid (GDCA); tauroursodeoxycholic acid (TUDCA); glycoursodeoxycholic acid (GUDCA); taurodeoxycholic acid (TDCA); glycolithocholic acid-3-sulfate (GLCA-3S); glycolithocholate (GLCA); taurohyodeoxycholic acid (THDCA); norcholic acid (NorCA);
conjugated forms of typical bile acids;
or combinations thereof.

9. The method of claim 6, further comprising:
administering to the subject a therapeutically effective amount of one or more primary or secondary bile acids and/or any pharmaceutically acceptable derivatives, esters, salts, solvates, hydrates, analogs, or prodrugs thereof; and/or
administering to the subject a therapeutically effective amount of one or more therapeutic agents capable of modulating (increasing or decreasing) the concentration levels or ratios of one or more primary or secondary bile acids, activating the endogenous production of one or more primary or secondary bile acids, and/or decreasing the breakdown of one or more primary or secondary bile acids; and/or
administering to the subject a therapeutically effective amount of one or more antidepressants selected from citalopram (Celexa®), escitalopram (Lexapro®), duloxetine (Cymbalta®), fluoxetine (Prozac®), paroxetine (Paxil®), sertraline (Zoloft®), trazodone (Desyrel®), lorazepam (Ativan®), oxazepam (Serax®), fluvoxamine (Luvox®), vilazodone (Viibryd®), vortioxetine (Trintellix®), aripiprazole (Abilify®), clozapine (Clozaril®), haloperidol (Haldol®), olanzapine (Zyprexa®), quetiapine (Seroquel®), risperidone (Risperdal®), ziprasidone (Geodon®), amitriptyline, amoxapine, desipramine (Norpramin®), doxepin, imipramine (Tofranil®), nortriptyline (Pamelor®), protriptyline, trimipramine, ketamine, or combinations thereof.

10. The method of claim 6, wherein the depression, anxiety, mood disorders, or neuropsychiatric symptoms are associated with neurological diseases or cognitive impairment, including dementia, vascular dementia, mixed dementia, early mild cognitive impairment (EMCI), late mild cognitive impairment (LMCI), Alzheimer's Disease, dementia with Lewy bodies, frontotemporal dementia, Creutzfeldt-Jakob disease, Parkinson's Disease, young-onset dementia, Korsakoff's syndrome, Huntington's disease, HIV-associated neurocognitive disorders, or other cognitive impairment disorders.

11. A method for detecting depression or anxiety in a subject, the method comprising:
analyzing a sample from a subject;
determining concentration levels or ratios of one or more biomarker metabolites or gut microbiome-related biomarker metabolites in the sample from the subject; and
determining the subject as having depression or anxiety or an increased risk of depression or anxiety when the concentration levels or ratios of the one or more biomarker metabolites or gut microbiome-related biomarker metabolites in the sample from the subject are different from (greater than or less than) the concentration levels or ratios of the one or more biomarker metabolites or gut microbiome-related biomarker metabolites in a control sample.

12. The method of claim 11, further comprising:
initially treating the subject for depression or anxiety by administering an effective amount sufficient to attenuate, reduce, or eliminate the symptoms of depression or anxiety of one or more of antidepressants, cognitive behavior therapy, exercise, dietary supplements, prebiotics, probiotics, dietary changes, or an elimination diet;
obtaining a second sample from the subject and determining the concentration levels or ratios of one or more biomarker metabolites or gut microbiome-related biomarker metabolites in the second sample from the subject;
evaluating the concentration levels or ratios of the one or more biomarker metabolites or gut microbiome-related biomarker metabolites in comparison to control concentration levels or ratios of the one or more biomarker metabolites or gut microbiome-related biomarker metabolites;
evaluating the efficacy of the depression or anxiety treatment; and
continuing the one or more initial depression or anxiety treatments; administering one or more additional depression or anxiety treatments; or administering one or more second depression or anxiety treatments (switching the treatment regimen).

13. (canceled)

14. The method of claim 11, further comprising:
administering to the subject a therapeutically effective amount of one or more primary or secondary bile acids and/or any pharmaceutically acceptable derivatives, esters, salts, solvates, hydrates, analogs, or prodrugs thereof; and/or
administering to the subject a therapeutically effective amount of one or more therapeutic agents capable of modulating (increasing or decreasing) the concentration levels or ratios of one or more primary or secondary bile acids, activating the endogenous production of one or more primary or secondary bile acids, and/or decreasing the breakdown of one or more primary or secondary bile acids.

15. The method of claim 11, wherein the biomarker metabolite or gut microbiome-related biomarker metabolite concentration level is greater than the control concentration level, wherein the biomarker metabolite or gut microbiome-related biomarker metabolite concentration level is greater than the control concentration level.

16. (canceled)

17. The method of claim 11, wherein two or more biomarker metabolite or gut microbiome-related biomarker metabolite concentration levels covary and are greater than the control concentration levels or covary and are less than the control concentration levels (positive correlation), or wherein two or more biomarker metabolite or gut microbiome-related biomarker metabolite concentration levels vary dissimilarly compared to the control concentration levels (negative correlation).

18. (canceled)

19. The method of claim 11, wherein the biomarker metabolite or gut microbiome-related biomarker metabolite comprises one or more primary bile acids selected from the group consisting of CDCA, CA, HCA, GHCA, and combinations thereof, wherein when the biomarker metabolite or gut microbiome-related biomarker metabolite comprises CDCA, the concentration levels in the sample from the subject are less than the concentration levels in the control sample.

20. (canceled)

21. The method of claim 11, wherein the biomarker metabolite or gut microbiome-related biomarker metabolite comprises one or more secondary bile acids selected from the group consisting of UDCA, LCA, 7-ketoLCA, HDCA, isoLCA, β-HDCA, alloLCA, dehydroLCA, 12-ketoLCA, LCA-3S, GLCA-3S, HCA, NorCA, 6,7-diketoLCA, GDCA, β-UCA, and combinations thereof, wherein when the biomarker metabolite or gut microbiome-related biomarker metabolite comprises one or more of LCA, isoLCA, alloLCA, 12-ketoLCA, LCA-3S, GLCA-3S, 7-ketoLCA, 6,7-diketoLCA, NorCA, GDCA, or β-HDCA, the concentration levels in the sample from the subject are greater than the concentration levels in the control sample, and wherein when the biomarker metabolite or gut microbiome-related biomarker metabolite comprises one or more of dehydroLCA, β-UCA, or HCA, the concentration levels in the sample from the subject are less than the concentration levels in the control sample.

22-23. (canceled)

24. The method of claim 11, wherein one or more ratios of the biomarker metabolites or gut microbiome-related biomarker metabolites are determined, the one or more ratios comprising a ratio of LCA/CDCA, 7-ketoLCA/CDCA, 12-ketoLCA/CDCA, GHCA/CDCA, HDCA/CDCA, ω-MCA/CDCA, alloLCA/CDCA, isoLCA/CDCA, LCA-3S/CDCA, GCA/TCA, TDCA/GDCA, THCA/GHCA, or combinations thereof, wherein when the ratio of the biomarker metabolites or gut microbiome-related biomarker metabolites comprises one or more of LCA/CDCA, 7-ketoLCA/CDCA, 12-ketoLCA/CDCA, GHCA/CDCA, HDCA/CDCA, ω-MCA/CDCA, alloLCA/CDCA, iSOLCA/CDCA, LCA-3S/CDCA, or GCA/TCA, the ratios in the sample from the subject are greater than the ratios in the control sample, and wherein when the ratio of the biomarker metabolites or gut microbiome-related biomarker metabolites comprises one or more of TDCA/GDCA or THCA/GHCA, the ratios in the sample from the subject are less than the ratios in the control sample.

25-26. (canceled)

27. The method of claim 11, wherein the sample from the subject is selected from one or more of whole blood, serum, plasma, urine, saliva, feces, or other body fluids.

28. The method of claim 11, wherein the control sample is from an untreated subject or a subject or a population of subjects not experiencing depression, anxiety, mood disorders, or neuropsychiatric symptoms or not at risk for depression, anxiety, mood disorders, or neuropsychiatric symptoms.

29. The method of claim 11, wherein the depression is Major Depression Disorder (MDD), core depression (CD+), anxious depression (ANX+), neurovegetative symptoms of melancholia (NVSM+), treatment resistant depression, subclinical characteristics associated with depression, or a neuropsychiatric symptom associated with a neurological disease or cognitive impairment.

30. (canceled)

31. The method of claim 12, wherein the antidepressant comprises citalopram (Celexa®), escitalopram (Lexapro®), duloxetine (Cymbalta®), fluoxetine (Prozac®), paroxetine (Paxil®), sertraline (Zoloft®), trazodone (Desyrel®), lorazepam (Ativan®), oxazepam (Serax®), fluvoxamine (Luvox®), vilazodone (Viibryd®), vortioxetine (Trintellix®), aripiprazole (Abilify®), clozapine (Clozaril®), haloperidol (Haldol®), olanzapine (Zyprexa®), quetiapine (Seroquel®), risperidone (Risperdal®), ziprasidone (Geodon®), amitriptyline, amoxapine, desipramine (Norpramin®), doxepin, imipramine (Tofranil®), nortriptyline (Pamelor®), protriptyline, trimipramine, ketamine, one or more other selective serotonin reuptake inhibitors (SSRIs), or combinations thereof.

32-34. (canceled)

35. The method of claim 12, wherein the efficacy of the depression treatment is evaluated using the Hamilton Depression Rating Scale (HRSD₁₇), the Quick Inventory of Depressive Symptomatology (QIDS), subscales thereof, or specific questions thereof.

36. A method for treating depression, anxiety, mood disorders, or neuropsychiatric symptoms in a subject the method comprising:
administering to the subject an effective amount sufficient to attenuate, reduce, or eliminate the symptoms of depression, anxiety, mood disorders, or neuropsychiatric symptoms of one or more of antidepressants, cognitive behavior therapy, exercise, dietary supplements, prebiotics, probiotics, dietary changes, or an elimination diet;
analyzing sample from the subject; and
measuring concentration levels or ratios in the subject's sample of one or more biomarker metabolites or gut microbiome-related biomarker metabolites comprising primary bile acids, secondary bile acids, or a combination thereof.

37. The method of claim 36, wherein the one or more biomarker metabolites or gut microbiome-related biomarker metabolites comprises one or more of:
Primary Bile Acids: cholic acid (CA); chenodeoxycholic acid (CDCA); chenodeoxycholic acid-24-acyl-β-d-glucuronide (CDCA-24G); 3β-cholic acid (β-CA); hyocholic acid (HCA); ω-monocarboxylic acid (ω-MCA); taurocholic acid (TCA); glycocholic acid (GCA); taurochenodeoxycholate (TCDCA); glycochenodeoxycholate (GCDCA); 5β-cholanic acid-3β,7β,12α-triol-5β-cholanic acid-3β, 7β, 12α-triol (β-UCA); taurohyocholate (THCA); glycohyocholate (GHCA);
Secondary Bile Acids: glycohyodeoxycholate (GHDCA); deoxycholic acid (DCA); 23-nordeoxycholic acid (NorDCA); β-ursodeoxycholic acid (UDCA); lithocholic acid (LCA); 6,7-diketolithocholic acid (6,7-diketoLCA); 7-ketolithocholic acid (7-ketoLCA); lithocholic acid-3-sulfate (LCA-3S); hyodeoxycholic acid (HDCA); isolithocholic acid (isoLCA); β-hyodeoxycholic acid (isohyodeoxycholic acid; β-HDCA); allolithocholic acid (isoallolithocholic acid; alloLCA); dehydroLCA; 12-ketodeoxycholic acid (12-ketoLCA); 33-ursodeoxycholic acid (isoursodeoxycholic acid; 3β-UDCA); glycodeoxycholic acid (GDCA); tauroursodeoxycholic acid (TUDCA); glycoursodeoxycholic acid (GUDCA); taurodeoxycholic acid (TDCA); glycolithocholic acid-3-sulfate (GLCA-3S); glycolithocholate (GLCA); taurohyodeoxycholic acid (THDCA); norcholic acid (NorCA);
conjugated forms of typical bile acids;
or combinations thereof.

38-45. (canceled)
